SEQUENCE LISTING (116 sequences)

SEQ ID NO: 1 - protein, 8 aa, Artificial Sequence
  Synthetic Construct
NANKGAMT

SEQ ID NO: 2 - protein, 5 aa, Artificial Sequence
  Synthetic Construct
IDKWG

SEQ ID NO: 3 - protein, 16 aa, Artificial Sequence
  Synthetic Construct
WRYSPVSINYGDSVKG

SEQ ID NO: 4 - protein, 7 aa, Artificial Sequence
  Synthetic Construct
VISGAWY

SEQ ID NO: 5 - protein, 7 aa, Artificial Sequence
  Synthetic Construct
FSSYAMG

SEQ ID NO: 6 - protein, 17 aa, Artificial Sequence
  Synthetic Construct
AITWSGDYTYYTDSVKG

SEQ ID NO: 7 - protein, 17 aa, Artificial Sequence
  Synthetic Construct
KFSQVSAFFATVSEYEY

SEQ ID NO: 8 - protein, 5 aa, Artificial Sequence
  Synthetic Construct
INTYL

SEQ ID NO: 9 - protein, 5 aa, Artificial Sequence
  Synthetic Construct
INTYL

SEQ ID NO: 10 - protein, 6 aa, Artificial Sequence
  Synthetic Construct
AINGAM

SEQ ID NO: 11 - protein, 6 aa, Artificial Sequence
  Synthetic Construct
ANNGAM

SEQ ID NO: 12 - protein, 16 aa, Artificial Sequence
  Synthetic Construct
AITSGGSINYADSVKG

SEQ ID NO: 13 - protein, 13 aa, Artificial Sequence
  Synthetic Construct
KAHPNPWGFDNDY

SEQ ID NO: 14 - protein, 5 aa, Artificial Sequence
  Synthetic Construct
INAMA

SEQ ID NO: 15 - protein, 16 aa, Artificial Sequence
  Synthetic Construct
SLKYDGSTNYADFAKG

SEQ ID NO: 16 - protein, 14 aa, Artificial Sequence
  Synthetic Construct
DMTDWGLVAGQFDV

SEQ ID NO: 17 - protein, 5 aa, Artificial Sequence
  Synthetic Construct
INAMG

SEQ ID NO: 18 - protein, 16 aa, Artificial Sequence
  Synthetic Construct
ALTSGGSTMYGDSVKG

SEQ ID NO: 19 - protein, 13 aa, Artificial Sequence
  Synthetic Construct
RLIRTINGVDYDY

SEQ ID NO: 20 - protein, 5 aa, Artificial Sequence
  Synthetic Construct
LYAIA

SEQ ID NO: 21 - protein, 19 aa, Artificial Sequence
  Synthetic Construct
CISATHGEDIVAYRESVKG

SEQ ID NO: 22 - protein, 26 aa, Artificial Sequence
  Synthetic Construct
DGATCGDLYGRSWDGGPVNRPPQFGS

SEQ ID NO: 23 - protein, 5 aa, Artificial Sequence
  Synthetic Construct
AYAMG

SEQ ID NO: 24 - protein, 17 aa, Artificial Sequence
  Synthetic Construct
SISWTGSTSYYADSVKG

SEQ ID NO: 25 - protein, 16 aa, Artificial Sequence
  Synthetic Construct
TTYFRGTYNVESAFGS

SEQ ID NO: 26 - protein, 5 aa, Artificial Sequence
  Synthetic Construct
SYVMG

SEQ ID NO: 27 - protein, 17 aa, Artificial Sequence
  Synthetic Construct
AIPWDDDWTRYEDFVSG

SEQ ID NO: 28 - protein, 10 aa, Artificial Sequence
  Synthetic Construct
STRPGAPYVY

SEQ ID NO: 29 - protein, 5 aa, Artificial Sequence
  Synthetic Construct
TYYMT

SEQ ID NO: 30 - protein, 17 aa, Artificial Sequence
  Synthetic Construct
AINTGGGTTYYADSVKG

SEQ ID NO: 31 - protein, 12 aa, Artificial Sequence
  Synthetic Construct
GDYYESTYYPRY

SEQ ID NO: 32 - protein, 114 aa, Artificial Sequence
  Synthetic Construct
VQLQESGGGLVQAGGSLRLSCAASGSLFAIDKWGWYRQVAGNERELVAWRYSPVSINYGDSVKGRFTVSGDSSRKRVYLQMNSLKPDDTAVYRCAAVISGAWYWGQGTQVTVSS

SEQ ID NO: 33 - protein, 114 aa, Artificial Sequence
  Synthetic Construct
VQLQESEGGWSGGGSASSDFDYDKMANANKGAMTGNADDAGNERELVAWRYSPVSINYGDSVKGRFTVSGDSSRKRVYLQMNSLKPDDTAVYRCAAVISGAWYWGQGTQVTVSS

SEQ ID NO: 34 - protein, 127 aa, Artificial Sequence
  Synthetic Construct
VQLQESGGGLVQTGGSLRLSCAASGRSFTFSSYAMGWFRHAPGKEREFLTAITWSGDYTYYTDSVKGRSTISRDNAKNTVYLQLNNLKPEDTAVYYCAAKFSQVSAFFATVSEYEYWGQGTQVTVSS

SEQ ID NO: 35 - protein, 120 aa, Artificial Sequence
  Synthetic Construct
VQLQESGGGLVQPGGSLRLSCAASGFTFSINTYLWYRQPPGKQREFVAAITSGGSINYADSVKGRFTISRDNAKKTVYLQMNSLKPEDTAVYVCNIKAHPNPWGFDNDYWGQGTQVTVSS

SEQ ID NO: 36 - protein, 119 aa, Artificial Sequence
  Synthetic Construct
VQLQESGGGLVQAGGSLRLSCAASGTFSINTYLWYRQPPGKQREFVAAITSGGSINYADSVKGRFTISRDNAKKTVYLQMNSLKPEDTAVYVCNIKAHPNPWGFDNDYWGQGTQVTVSS

SEQ ID NO: 37 - protein, 119 aa, Artificial Sequence
  Synthetic Construct
VQLQESGGGLVQPGGSLRLSCAASGTFTINTYLWYRQPPGKQREFVAAITRGGSINYADSVKGRFTISRDNAKKTVYLQMNSLKPEDTAVYVCNIKAHPNPWGFDNDYWGQGTQVTVSS

SEQ ID NO: 38 - protein, 119 aa, Artificial Sequence
  Synthetic Construct
VQLQESEGGWSGGGSGSGDFGYDKMANAINGAMTDNADAGKQREFVAAITSGGSINYADSVKGRFTISTDNAKKTVYLQMNSLKPEDTAVYVCNIKAHPNPWGFDNDYWGQGTPATASS

SEQ ID NO: 39 - protein, 119 aa, Artificial Sequence
  Synthetic Construct
VQLQESEGGWSGGGSGSGDFDYEKMANANNGAMTDNADAGKQREFVAAITSGGSINYADSVKGRFTISRDNTKKTVYLQMNSLKPEDTAVYVCNIKAHPNPWGFDNDYWGQGTQVTVSS

SEQ ID NO: 40 - protein, 121 aa, Artificial Sequence
  Synthetic Construct
VQLQESGGGEVQPGGSLRLSCAASGHSFNINAMAWFRQAPDKQRELVASLKYDGSTNYADFAKGRFTISRGDAKDSIVLQLNSLKPEDTAVYRCAADMTDWGLVAGQFDVWGRGTQVTVSS

SEQ ID NO: 41 - protein, 120 aa, Artificial Sequence
  Synthetic Construct
VQLQESGGGLVQPGGSLRLSCSASGSIFSINAMGWYRQAPGKQRELVAALTSGGSTMYGDSVKGRFTISRDGSKKMVYLQMDSLKPEDTAVYYCASRLIRTINGVDYDYRGQGTQVTVSS

SEQ ID NO: 42 - protein, 136 aa, Artificial Sequence
  Synthetic Construct
VQLQESGGGLVQPGGSLRLSCTAPGVTLDLYAIAWFRQAPGKEREGVSCISATHGEDIVAYRESVKGRFTIFRDKTENAVYLQMNNLRPEDTAVYYCATDGATCGDLYGRSWDGGPVNRPPQFGSWGQGTQVTVSS

SEQ ID NO: 43 - protein, 124 aa, Artificial Sequence
  Synthetic Construct
VQLQESGGGSVQAGDSLTLSCSASGPAGSAYAMGWFRQPPGKEREFVASISWTGSTSYYADSVKGRFSVSIVNANNTMLLEMKSLKPEDTAVYVCAATTYFRGTYNVESAFGSWGQGKQVTVSS

SEQ ID NO: 44 - protein, 118 aa, Artificial Sequence
  Synthetic Construct
VQLQESGGGLVQTGGSLRLSCAASGRTTSSYVMGWFRQAPGKGRVFVAAIPWDDDWTRYEDFVSGRFTISIDNAKNTVYLQMNSLKPDDTAVYYCAASTRPGAPYVYWGQGTQVTVSS

SEQ ID NO: 45 - protein, 120 aa, Artificial Sequence
  Synthetic Construct
VQLQESGGGLVQPGGSLRLSCAASGFTFSTYYMTWVRQAPGKGLEWISAINTGGGTTYYADSVKGRFTISRDNAKNTLYLQMNSLKPEDTAVYYCTIGDYYESTYYPRYWGQGTQVTVSS

SEQ ID NO: 46 - protein, 115 aa, Artificial Sequence
  Synthetic Construct
QVQLVESGGGLVQPGGSLRLSCAASGSLFAIDKWGWFRQAPGKEREIVSWRYSPVSINYGDSVKGRFTISRDNSKNTLYLQMNSLRAEDTAVYYCAAVISGAWYWGQGTLVTVSS

SEQ ID NO: 47 - protein, 115 aa, Artificial Sequence
  Synthetic Construct
EVQLLESGGGLVQPGGSLRLSCAASGSLFAIDKWGWVRQAPGKGLEWVSWRYSPVSINYGDSVKGRFTISRDNSKNTLYLQMNSLRAEDTAVYYCAAVISGAWYWGQGTLVTVSS

SEQ ID NO: 48 - protein, 115 aa, Artificial Sequence
  Synthetic Construct
QVQLQESGGGLVQPGGSLRLSCAASGSLFAIDKWGWFRQAPGKEREIVSWRYSPVSINYGDSVKGRFTISGDNSKNTLYLQMNSLRAEDTAVYRCAAVISGAWYWGQGTQVTVSS

SEQ ID NO: 49 - protein, 128 aa, Artificial Sequence
  Synthetic Construct
QVQLVESGGGLVQPGGSLRLSCAASGRSFTFSSYAMGWFRQAPGKEREIVSAITWSGDYTYYTDSVKGRFTISRDNSKNTLYLQMNSLRAEDTAVYYCARKFSQVSAFFATVSEYEYWGQGTLVTVSS

SEQ ID NO: 50 - protein, 128 aa, Artificial Sequence
  Synthetic Construct
EVQLLESGGGLQQPGGSLRLSCAASGRSFTFSSYAMGWVRQAPGKGLEWVSAITWSGDYTYYTDSVKGRFTISRDNSKNTLYLQMNSLRAEDTAVYYCARKFSQVSAFFATVSEYEYWGQGTMVTVSS

SEQ ID NO: 51 - protein, 128 aa, Artificial Sequence
  Synthetic Construct
EVQLQESGGGLQQPGGSLRLSCAASGRSFTFSSYAMGWVRQAPGKGLEWVSAITWSGDYTYYTDSVKGRFTISRDNSKNTLYLQMNSLRAEDTAVYYCAAKFSQVSAFFATVSEYEYWGQGTQVTVSS

SEQ ID NO: 52 - protein, 5 aa, Artificial Sequence
  Synthetic Construct
INTYL

SEQ ID NO: 53 - protein, 6 aa, Artificial Sequence
  Synthetic Construct
  VARIANT 2: Xaa = N or I
AXNGAM

SEQ ID NO: 54 - protein, 1 aa, Artificial Sequence
  Synthetic Construct
  VARIANT 1: Can be present in repeats of any integer
G

SEQ ID NO: 55 - protein, 16 aa, Artificial Sequence
  Synthetic Construct
  VARIANT (3)..(16): Can be present or absent
GSGSGSGSGSGSGSGS

SEQ ID NO: 56 - protein, 40 aa, Artificial Sequence
  Synthetic Construct
  VARIANT (6)..(40): Can be present or absent
GSGGSGSGGSGSGGSGSGGSGSGGSGSGGSGSGGSGSGGS

SEQ ID NO: 57 - protein, 40 aa, Artificial Sequence
  Synthetic Construct
  VARIANT (6)..(40): Can be present or absent
GGGGSGGGGSGGGGSGGGGSGGGGSGGGGSGGGGSGGGGS

SEQ ID NO: 58 - protein, 32 aa, Artificial Sequence
  Synthetic Construct
  VARIANT (5)..(32): Can be present or absent
GGGSGGGSGGGSGGGSGGGSGGGSGGGSGGGS

SEQ ID NO: 59 - protein, 15 aa, Artificial Sequence
  Synthetic Construct
GGGGSGGGGSGGGGS

SEQ ID NO: 60 - protein, 30 aa, Artificial Sequence
  Synthetic Construct
GGGGSGGGGSGGGGSGGGGSGGGGSGGGGS

SEQ ID NO: 61 - protein, 15 aa, Artificial Sequence
  Synthetic Construct
  VARIANT (1)..(15): Can be present in repeats of any integer up to 3
GSTSGSGKPGSGEGS

SEQ ID NO: 62 - protein, 5 aa, Artificial Sequence
  Synthetic Construct
DTYMY

SEQ ID NO: 63 - protein, 17 aa, Artificial Sequence
  Synthetic Construct
RIDPANDNTKYAQKFQG

SEQ ID NO: 64 - protein, 10 aa, Artificial Sequence
  Synthetic Construct
AKNLLNYFDY

SEQ ID NO: 65 - protein, 11 aa, Artificial Sequence
  Synthetic Construct
RASQEISGYLS

SEQ ID NO: 66 - protein, 7 aa, Artificial Sequence
  Synthetic Construct
ATSTLQS

SEQ ID NO: 67 - protein, 9 aa, Artificial Sequence
  Synthetic Construct
LQYAIYPLT

SEQ ID NO: 68 - protein, 119 aa, Artificial Sequence
  Synthetic Construct
QVQLVQSGAEVKKPGASVKVSCKASGFNIKDTYMYWVRQAPGQGLEWMGRIDPANDNTKYAQKFQGRVTITADTSTSTAYMELSSLRSEDTAVYYCARAKNLLNYFDYWGQGTLVTVSS

SEQ ID NO: 69 - protein, 119 aa, Artificial Sequence
  Synthetic Construct
QVQLVQSGAEVKKPGASVKVSCKASGFNIKDTYMYWVRQAPGQGLEWIGRIDPANDNTKYAPKFQGRVTITADTSTNTAYMELSSLRSEDTAVYYCARAKNLLNYFDYWGQGTLVTVSS

SEQ ID NO: 70 - protein, 119 aa, Artificial Sequence
  Synthetic Construct
EVQLVQSGAEVKKPGASVKVSCKASGFNIKDTYMYWVRQAPGQGLEWMGRIDPANDNTKYAQKFQGRVTITADTSTNTAYMELSSLRSEDTAVYYCARAKNLLNYFDYWGQGTLVTVSS

SEQ ID NO: 71 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSGSGTDFTLTISSLQPEDFATYYCLQYAIYPLTFGQGTKLEIKR

SEQ ID NO: 72 - protein, 108 aa, Artificial Sequence
  Synthetic Construct
DIQMTQSPSSLSASVGDRVTITCRASQEISGYLSWLQQKPGKAPKRLIYATSTLQSGVPSRFSGSRSGTDYTLTISSLQPEDFATYYCLQYAIYPLTFGQGTKLEIKR

SEQ ID NO: 73 - protein, 108 aa, Artificial Sequence
  Synthetic Construct
DIQMTQSPSSLSASVGDRVTITCRASQEISGYLSWYQQKPGKAPKRLIYATSTLDSGVPSRFSGSRSGSDYTLTISSLQPEDFATYYCLQYAIYPLTFGQGTKLEIKR

SEQ ID NO: 74 - protein, 8 aa, Artificial Sequence
  Synthetic Construct
GFTFSSYT

SEQ ID NO: 75 - protein, 8 aa, Artificial Sequence
  Synthetic Construct
ISHGGGDT

SEQ ID NO: 76 - protein, 16 aa, Artificial Sequence
  Synthetic Construct
ARHSGYERGYYYVMDY

SEQ ID NO: 77 - protein, 10 aa, Artificial Sequence
  Synthetic Construct
ESVDYYGFSF

SEQ ID NO: 78 - protein, 3 aa, Artificial Sequence
  Synthetic Construct
AAS

SEQ ID NO: 79 - protein, 8 aa, Artificial Sequence
  Synthetic Construct
QQSKEVPW

SEQ ID NO: 80 - protein, 8 aa, Artificial Sequence
  Synthetic Construct
GYTFTSYT

SEQ ID NO: 81 - protein, 8 aa, Artificial Sequence
  Synthetic Construct
INPTTGYT

SEQ ID NO: 82 - protein, 10 aa, Artificial Sequence
  Synthetic Construct
ARDDAYYSGY

SEQ ID NO: 83 - protein, 7 aa, Artificial Sequence
  Synthetic Construct
ENIYSNL

SEQ ID NO: 84 - protein, 3 aa, Artificial Sequence
  Synthetic Construct
AAK

SEQ ID NO: 85 - protein, 9 aa, Artificial Sequence
  Synthetic Construct
QHFWGTPWT

SEQ ID NO: 86 - protein, 8 aa, Artificial Sequence
  Synthetic Construct
GFAFSSYD

SEQ ID NO: 87 - protein, 8 aa, Artificial Sequence
  Synthetic Construct
ITIGGGTT

SEQ ID NO: 88 - protein, 12 aa, Artificial Sequence
  Synthetic Construct
ARHRYDYFAMDN

SEQ ID NO: 89 - protein, 10 aa, Artificial Sequence
  Synthetic Construct
ENVDNYGINF

SEQ ID NO: 90 - protein, 3 aa, Artificial Sequence
  Synthetic Construct
VSS

SEQ ID NO: 91 - protein, 8 aa, Artificial Sequence
  Synthetic Construct
QQSKDVPW

SEQ ID NO: 92 - protein, 135 aa, Mus musculus
SQVQLQQSGAELARPGASVKMSCKASGYTFTSYTMHWVKQRPGQGLEWIGYINPTTGYTNYNQKFKDKANPTTGYTNYNQKFKDKATLTADKSSSTAYMQLSSLTSEDSAVYYCARDDAYYSGYWGQGTTLTVSS

SEQ ID NO: 93 - protein, 108 aa, Mus musculus
DIQMTQSPASLSVSVGETVTITCRASENIYSNLAWYRQKQGKSPQLLVYAAKNLADGVPSRFSGSGSGTQYSLKINSLQSEDFGSYYCQHFWGTPWTFGGGTKLEIKR

SEQ ID NO: 94 - protein, 140 aa, Mus musculus
VQLVESGGGLVKPGGSLKLSCAASGFAFSSYDMSWVRQTPEKRLVWVAYITIGGGTTYYSDTVKRLVWVAYITIGGGTTYYSDTVKGRFTISRDNAKNTLYLQMSSLKSEDTAMYYCARHRYDYFAMDNWGHGTSVTVSS

SEQ ID NO: 95 - protein, 112 aa, Mus musculus
DIVLTQSPASLAVSLEHRATISCQASENVDNYGINFMNWFQHKPAQPPQLLIYVSSNLGSGVPAKFSGSGSGTDFSLNIHPMEEDDTAMYFCQQSKDVPWTFSGGTKLEIKR

SEQ ID NO: 96 - protein, 123 aa, Mus musculus
EVKLVESGGGLVQPGGSLKLSCAASGFTFSSYTMSWIRQTPEKRLEWVAYISHGGGDTYYPDTVKGRFTISRDNAKNTLYLQMSSLKSEDTAMYYCARHSGYERGYYYVMDYWGQGTSVTVSS

SEQ ID NO: 97 - protein, 111 aa, Mus musculus
DIVLTQFPTSLAVSLGQRATISCRASESVDYYGFSFINWFQQKPGQPPKLLIYAASNQGSGVPARFGGSGSGTDFSLNIHPMEEDDTAMYFCQQSKEVPWTFGGGTKLEIK

SEQ ID NO: 98 - protein, 203 aa, Artificial Sequence
  Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVH

SEQ ID NO: 99 - protein, 10 aa, Artificial Sequence
  Synthetic Construct
GYTFTNYGMN

SEQ ID NO: 100 - protein, 17 aa, Artificial Sequence
  Synthetic Construct
WINTYTGEPTYAADFKR

SEQ ID NO: 101 - protein, 14 aa, Artificial Sequence
  Synthetic Construct
YPHYYGSSHWYFDV

SEQ ID NO: 102 - protein, 11 aa, Artificial Sequence
  Synthetic Construct
SASQDISNYLN

SEQ ID NO: 103 - protein, 7 aa, Artificial Sequence
  Synthetic Construct
FTSSLHS

SEQ ID NO: 104 - protein, 9 aa, Artificial Sequence
  Synthetic Construct
QQYSTVPWT

SEQ ID NO: 105 - protein, 5 aa, Artificial Sequence
  Synthetic Construct
SYSMN

SEQ ID NO: 106 - protein, 17 aa, Artificial Sequence
  Synthetic Construct
SISSSSSYIYYADSVKG

SEQ ID NO: 107 - protein, 7 aa, Artificial Sequence
  Synthetic Construct
VTDAFDI

SEQ ID NO: 108 - protein, 11 aa, Artificial Sequence
  Synthetic Construct
RASQGIDNWLG

SEQ ID NO: 109 - protein, 7 aa, Artificial Sequence
  Synthetic Construct
DASNLDT

SEQ ID NO: 110 - protein, 9 aa, Artificial Sequence
  Synthetic Construct
QQAKAFPPT

SEQ ID NO: 111 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 112 - protein, 10 aa, Artificial Sequence
  Synthetic Construct
GSLFAIDKWG

SEQ ID NO: 113 - protein, 9 aa, Artificial Sequence
  Synthetic Construct
AAVISGAWY

SEQ ID NO: 114 - protein, 12 aa, Artificial Sequence
  Synthetic Construct
GRSFTFSSYAMG

SEQ ID NO: 115 - protein, 18 aa, Artificial Sequence
  Synthetic Construct
AKFSQVSAFFATVSEYEY

SEQ ID NO: 116 - protein, 19 aa, Artificial Sequence
  Synthetic Construct
ARKFSQVSAFFATVSEYEY

(19) United States
(12) Patent Application Publication
(10) Pub. No.: US 2024/0150466 A1
(43) Pub. Date: May 9, 2024

(54) ANTI-IGFBP7 CONSTRUCTS AND USES THEREOF

(71) Applicant: DYNAMICURE BIOTECHNOLOGY LLC, Waltham, MA (US)

(72) Inventors: Zirong CHEN, Saugus, MA (US); Jian LI, Chestnut Hill, MA (US); Angela NORTON, Reading, MA (US); Shuo WANG, Lexington, MA (US); Zhinan XIA, Waltham, MA (US)

(73) Assignee: DYNAMICURE BIOTECHNOLOGY LLC, Waltham, MA (US)

(21) Appl. No.: 18/282,201

(22) PCT Filed: Mar. 25, 2022
(86) PCT No.: PCT/US2022/071354

§ 371 Date: Sep. 14, 2023

Related U.S. Application Data
(60) Provisional application No. 63/166,146, filed on Mar. 25, 2021.

(51) Int. Cl. C07K 16/28; A61K 45/06; A61P 35/00; A61K 39/00

(52) U.S. Cl. CPC C07K 16/2818; A61K 45/06; A61P 35/00; C07K 16/2827; A61K 2039/505

(57) ABSTRACT

The present application provides anti-IGFBP7 constructs that bind to IGFBP7 (e.g., anti-IGFBP7 antibodies), nucleic acid molecules encoding an amino acid sequence of the anti-IGFBP7, vectors comprising the nucleic acid molecules, host cells containing the vectors, methods of preparing the anti-IGFBP7 construct, pharmaceutical compositions containing the anti-IGFBP7 construct, and methods of using the anti-IGFBP7 construct or compositions.

### CROSS-REFERENCE TO RELATED APPLICATION

This application claims priority to U.S. provisional application 63/166,146, filed on Mar. 25, 2021, the content of which is incorporated by reference in their entirety for all purposes.

### TECHNICAL FIELD

The present disclosure relates to anti-IGFBP7 constructs (such as anti-IGFBP7 antibodies) and the uses thereof.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 193852000540SEQLIST.TXT, date recorded: Mar. 24, 2022, size: 56,698 bytes).

### BACKGROUND OF THE APPLICATION

IGFBP7 (Insulin-like Growth Factor Binding Protein 7) is a protein that in humans is encoded by the IGFBP7 gene. Insulin-like growth factors (IGFs) are proteins that are involved in promoting cellular growth and division and preventing premature apoptosis. The major function of IGFBP7 is to regulate the availability of IGFs in body fluids and tissues, as well as modulate the binding of IGFs to receptors. IGFBP7 is active in the lining of blood vessels; its interaction with IGFs and IGF receptors is crucial to control BRAF signaling, which is involved in directing cell growth. These processes have implicated IGFBP7 in cellular adhesion and cancer.

The disclosures of all publications, patents, patent applications and published patent applications referred to herein are hereby incorporated herein by reference in their entirety.

### BRIEF SUMMARY OF THE APPLICATION

The following summary is illustrative only and is not intended to be limiting in any way. That is, the following summary is provided to introduce highlights, benefits and advantages of the novel molecules and the uses thereof. Thus, the following summary is not intended to identify essential features of the claimed subject matter, nor is it intended for use in determining the scope of the claimed subject matter.

The present application in on aspect provides an anti-IGFBP7 construct comprising a polypeptide comprising a single domain antibody (sdAb) moiety specifically recognizing IGFBP7, wherein the sdAb moiety comprises:
1) a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
2) a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
3) a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs;
4) a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
5) a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
6) a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
7) a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
8) a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs;
9) a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs;
10) a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs;
11) a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs;
12) a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs; or
13) a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs.

The present application in another aspect provides an anti-IGFBP7 construct comprising a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in any of SEQ ID NOs: 32-51.

In some embodiments according to any of the anti-IGFBP7 constructs described above, the sdAb moiety comprises the amino acid sequence of any one of SEQ ID NOs: 32-51, or a variant thereof having at least about 80% sequence identify to any one of SEQ ID NOs: 32-51.

In some embodiments according to any of the anti-IGFBP7 constructs described above, the sdAb moiety is camelid, chimeric, human, partially humanized, or fully humanized.

In some embodiments according to any of the anti-IGFBP7 constructs described above, the sdAb moiety is a V_{H}H antibody.

In some embodiments according to any of the anti-IGFBP7 constructs described above, anti-IGFBP7 construct blocks the binding of CD93 to IGFBP7. In some embodiments, the CD93 is human CD93.

In some embodiments according to any of the anti-IGFBP7 constructs described above, the IGFBP7 is human IGFBP7.

In some embodiments according to any of the anti-IGFBP7 constructs described above, the anti-IGFBP7 construct further comprises a second moiety. In some embodiments, the second moiety comprises an antibody moiety that specifically recognizes an antigen. In some embodiments, the antigen is PD-L1. In some embodiments, the antibody moiety is a full-length antibody, a Fab, a Fab′, a (Fab′)2, an Fv, a single chain Fv (scFv), a scFv-scFv, a minibody, a diabody, or an sdAb.

In some embodiments according to any of the anti-IGFBP7 constructs described above, the anti-IGFBP7 construct further comprises a second moiety that comprises a half-life extending moiety.

In some embodiments according to any of the anti-IGFBP7 constructs described above, the anti-IGFBP7 construct is an antibody-drug conjugate.

The present application in another aspect provides an anti-IGFBP7 construct that specifically binds to IGFBP7 competitively with the anti-IGFBP7 construct of any one of anti-IGFBP7 constructs described above.

The present application in another aspect provides a pharmaceutical composition comprising any of the anti-IGFBP7 constructs described above and a pharmaceutical acceptable carrier.

The present application in another aspect provides a polynucleotide encoding the polypeptide of any of the anti-IGFBP7 constructs described above or a portion thereof.

The present application in another aspect provides a nucleic acid construct, comprising any of the polynucleotides described above, and optionally further comprising a promoter in operative connection with the polynucleotide.

The present application in another aspect provides a vector comprising any of the nucleic acid constructs described above.

The present application in another aspect provides an isolated host cell comprising any of the polynucleotides, nucleic acid constructs, or vectors described above.

The present application in another aspect provides a culture medium comprising the polypeptide of any of the anti-IGFBP7 constructs, polynucleotides, nucleic acid constructs, vectors or host cells described above.

The present application in another aspect provides a method of producing an anti-IGFBP7 construct, comprising: a) culturing any of the isolated host cells described above under conditions effective to express the polypeptide, and b) obtaining the polypeptide from the host cell.

The present application in another aspect provides a method of treating a disease or condition (such as a cancer, such as a solid tumor) or inhibiting abnormal vessel growth in a tissue comprising administering to the individual an effective mount of any of the anti-IGFBP7 constructs or pharmaceutical compositions described above. In some embodiments, the disease of condition is associated with an abnormal vascular structure. In some embodiments, the disease or condition is a cancer. In some embodiments, the cancer is a solid tumor. In some embodiments, the cancer comprises CD93+ endothelial cells. In some embodiments, the cancer comprises IGFBP7+ blood vessels. In some embodiments, the cancer is characterized by tumor hypoxia. In some embodiments, the cancer is a locally advanced or metastatic cancer. In some embodiments, the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer. In some embodiments, the anti-IGFBP7 construct is administered parenterally into the individual. In some embodiments, the method further comprises administering a second therapy. In some embodiments, the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy. In some embodiments, the second therapy is an immunotherapy. In some embodiments, the immunotherapy comprises administering an immunomodulatory agent. In some embodiments, the immunomodulatory agent is an immune checkpoint inhibitor. In some embodiments, the immune checkpoint inhibitor comprises an anti-PD-L1 antibody or an anti-PD-1 antibody. In some embodiments, the individual is a human.

### BRIEF DESCRIPTION OF THE DRAWINGS

FIG. 1 shows anti-human IGFBP7 antibody titers in llama serum after immunizing a llama with human IGFBP7.

FIGS. 2A-2B show binding of various anti-IGFBP7 nanobodies to human IGFBP7-expressing (hIGFBP7) HEK293T cells.

FIGS. 3A-3B show binding of various anti-IGFBP7 nanobodies to mouse IGFBP7-expressing (mIGFBP7) HEK293T cells.

FIG. 4 shows binding of various anti-IGFBP7 nanobody Fc-fusion constructs to human IGFBP7-expressing (hIGFBP7) HEK293T cells.

FIG. 5 shows binding of various anti-IGFBP7 nanobody Fc-fusion constructs to mouse IGFBP7-expressing (mIGFBP7) CHO-K1 cells.

FIG. 6 shows binding affinity of various anti-IGFBP7 nanobody Fc-fusion constructs against human IGFBP7.

FIG. 7 shows binding affinity of various anti-IGFBP7 nanobody Fc-fusion constructs against mouse IGFBP7.

FIG. 8 shows a schematic representation of two anti-IGFBP7 bispecific molecules.

FIG. 9 shows binding affinity of various monospecific and bispecific anti-IGFBP7 nanobody Fc-fusion constructs against human IGFBP7.

FIG. 10 shows binding affinity of various monospecific and bispecific anti-IGFBP7 nanobody Fc-fusion constructs against mouse IGFBP7.

FIG. 11 shows binding affinity of various monospecific and bispecific anti-IGFBP7 nanobody Fc-fusion constructs against cynomolgus IGFBP7.

FIGS. 12A-12B show binding of various monospecific and bispecific anti-IGFBP7 nanobody Fc-fusion constructs to human IGFBP7 displayed on the cell surface.

FIG. 13 shows binding of various bispecific anti-IGFBP7 nanobody Fc-fusion constructs to human and mouse IGFBP7-expressing HEK293T cells.

FIG. 14 shows the effects of blocking the interaction between CD93 and IGFBP7 by various anti-IGFBP7 nanobodies in human CD93-expressing CHO cells.

FIGS. 15A-15C show the effects of blocking the interaction between CD93 and IGFBP7 by various anti-IGFBP7 nanobody Fc-fusion constructs in human CD93-expressing CHO cells.

FIG. 16 shows the effects of blocking the interaction between CD93 and IGFBP7 by various monospecific and bispecific anti-IGFBP7 nanobody Fc-fusion constructs in human CD93-expressing CHO cells.

FIGS. 17A-17B show the inhibition of HUVEC tube formation by various anti-IGFBP7 nanobody Fc-fusion constructs as compared to control.

FIG. 18 shows epitope binning of anti-IGFBP7 antibodies by Octet competition.

### DETAILED DESCRIPTION OF THE APPLICATION

The present application provides novel anti-IGFBP7 constructs that specifically bind to IGFBP7 (such as anti-IGFBP7 monoclonal or multispecific antibodies), methods of preparing the anti-IGFBP7 constructs, methods of using the constructs (e.g., methods of treating a disease or condition).

### I. Definitions

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antibody moiety” refers to a full-length antibody or an antigen-binding fragment thereof.

A full-length antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, sdAbs are engineered from camelid HCAbs, and their heavy chain variable domains are referred herein as “V_{H}Hs”. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above-cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra

⁵Residue numbering follows the nomenclature of Honegger and Plückthun, supra

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

“Framework” or “FR” residues are those variable-domain residues other than the CDR residues as herein defined.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, See Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

“Homologous” refers to the sequence similarity or sequence identity between two polypeptides or between two nucleic acid molecules. When a position in both of the two compared sequences is occupied by the same base or amino acid monomer subunit, e.g., if a position in each of two protein molecules is occupied by lysine, or if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the protein sequences SGTSTD and TGTSDA share 50% homology. Generally, a comparison is made when two sequences are aligned to give maximum homology.

The term “constant domain” refers to the portion of an immunoglobulin molecule having a more conserved amino acid sequence relative to the other portion of the immunoglobulin, the variable domain, which contains the antigen-binding site. The constant domain contains the C_{H}1, C_{H}2 and C_{H}3 domains (Collectively, C_{H}) of the heavy chain and the CHL (or C_{L}) domain of the light chain.

The “light chains” of antibodies (immunoglobulins) from any mammalian species can be assigned to one of two clearly distinct types, called kappa (“κ”) and lambda (“λ”), based on the amino acid sequences of their constant domains.

The “CH1 domain” (also referred to as “C1” of “H1” domain) usually extends from about amino acid 118 to about amino acid 215 (EU numbering system).

“Hinge region” is generally defined as a region in IgG corresponding to Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” domain) comprises the stretch of residues C-terminal to a CH2 domain in an Fc region (i.e. from about amino acid residue 341 to the C-terminal end of an antibody sequence, typically at amino acid residue 446 or 447 of an IgG).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

“Fc receptor” or “FcR” describes a receptor that binds the Fc region of an antibody. The preferred FcR is a native sequence human FcR. Moreover, a preferred FcR is one which binds an IgG antibody (a gamma receptor) and includes receptors of the FcγRI, FcγRII, FcRN, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See M. Daëron, Annu. Rev. Immunol. 15:203-234 (1997). FcRN is critical to the recycling of an antibody to the blood allowing for increased serum half-life of the antibodies. FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody or fragment thereof “competes” for binding to a target antigen with a second antibody or fragment thereof when the first antibody or fragment thereof inhibits the target antigen binding of the second antibody of fragment thereof by at least about 50% (such as at least about any one of 55%, 60%, 65%, 70%, 75%, 80%, 85%, 90%, 95%, 98% or 99%) in the presence of an equimolar concentration of the first antibody or fragment thereof, or vice versa. A high throughput process for “binning” antibodies based upon their cross-competition is described in PCT Publication No. WO 03/48731.

As used herein, the terms “specifically binds,” “specifically recognizing,” and “is specific for” refer to measurable and reproducible interactions, such as binding between a target and an antibody or antibody moiety, which is determinative of the presence of the target in the presence of a heterogeneous population of molecules, including biological molecules. For example, an antibody or antibody moiety that specifically recognizes a target (which can be an epitope) is an antibody or antibody moiety that binds this target with greater affinity, avidity, more readily, and/or with greater duration than its bindings to other targets. In some embodiments, the extent of binding of an antibody to an unrelated target is less than about 10% of the binding of the antibody to the target as measured, e.g., by a radioimmunoassay (RIA). In some embodiments, an antibody that specifically binds a target has a dissociation constant (K_{D}) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA-, RIA-, ECL-, IRMA-, EIA-, BLI, SPR, BIACORE™-tests and peptide scans.

A “bispecific” or “multispecific” antibody can refer to an antibody that binds to two or more different antigens, or an antibody that binds to two or more different epitopes of a same antigen.

An “isolated” antibody (or construct) is one that has been identified, separated and/or recovered from a component of its production environment (e.g., natural or recombinant).

Preferably, the isolated polypeptide is free of association with all other components from its production environment.

An “isolated” nucleic acid molecule encoding a construct, antibody, or antigen-binding fragment thereof described herein is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies described herein is in a form other than in the form or setting in which it is found in nature. Isolated nucleic acid molecules therefore are distinguished from nucleic acid encoding the polypeptides and antibodies described herein existing naturally in cells. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, and may contain mutations. Mutant progeny that have the same function or biological activity as screened or selected for in the originally transformed cell are included herein.

The term “immunoconjugate” includes reference to a covalent linkage of a therapeutic agent or a detectable label to an antibody such as an antibody moiety described herein. The linkage can be direct or indirect through a linker (such as a peptide linker).

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods of the application contemplate any one or more of these aspects of treatment.

In the context of cancer, the term “treating” includes any or all of: inhibiting growth of cancer cells, inhibiting replication of cancer cells, lessening of overall tumor burden and ameliorating one or more symptoms associated with the disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to that of a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference may be obtained from a healthy and/or non-diseased sample. In some examples, a reference may be obtained from an untreated sample. In some examples, a reference is obtained from a non-diseased or non-treated sample of an individual. In some examples, a reference is obtained from one or more healthy individuals who are not the individual or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing” as used herein, includes providing prophylaxis with respect to the occurrence or recurrence of a disease in an individual that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

The terms “subject,” “individual,” and “patient” are used interchangeably herein to refer to a mammal, including, but not limited to, human, bovine, horse, feline, canine, rodent, or primate. In some embodiments, the individual is a human.

An “effective amount” of an agent refers to an amount effective, at dosages and for periods of time necessary, to achieve the desired therapeutic or prophylactic result. The specific dose may vary depending on one or more of: the particular agent chosen, the dosing regimen to be followed, whether it is administered in combination with other compounds, timing of administration, the tissue to be imaged, and the physical delivery system in which it is carried.

The terms “pharmaceutical formulation” and “pharmaceutical composition” refer to a preparation which is in such form as to permit the biological activity of the active ingredient(s) to be effective, and which contains no additional components which are unacceptably toxic to an individual to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to an individual. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

It is understood that embodiments of the application described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

### II. Anti-IGFBP7 Constructs

The present application provides anti-IGFBP7 constructs comprising an anti-IGFBP7 antibody moiety that specifically binds to IGFBP7 as described herein.

### IGFBP7

Insulin-like growth factor (IGF)-binding protein (IGFBP) 7, also known as Mac25, IGFBP-rp1, tumor-derived adhesion factor (TAF), prostacyclin-stimulating factor (PSF), and angiomodulin (AGM), is a secreted extracellular matrix (ECM) protein belonging to IGFBP family. See Hwa et al. Endocr Rev. 1999; 20(6):761-87; Bach et al. Endocrinology. 2018; 159(2):570-8. Members of IGFBP family contain an IGF-binding (IB) domain at the N-terminus which binds to IGF1 and helps to modulate the bioavailability of IGF1 in the blood. IGFBP7 lacks the C-terminal domain, which functions to stabilize IGF1 binding, thus its affinity for IGF-1 is significantly lower than that of IGFBP1-6. See Oh et al. J Biol Chem. 1996; 271(48):30322-5. IGFBP7 was found to be expressed in many normal tissues and cancer cells; however, the exact role of IGFBP7 in cancer was controversial. On one hand, IGFBP7 was shown to be released from cancer cells, and to act as a tumor suppressor to trigger tumor apoptosis and suppress angiogenesis (Wajapeyee et al. Cell. 2008; 132(3):363-74); IGF1R was proposed as the receptor and IGFBP7 binding blocked the interaction between IGF-1 and IGF1R to inhibit expansion and aggressiveness of cancer stem-like cell. See Cao et al. Cancer Cell. 2017; 31(1):110-26; Evdokimova et al. Sci Signal. 2012; 5(255):ra92. Administration of IGFBP7 inhibited tumor growth in vivo, and IGFBP7−/− mice were susceptible to diethylnitrosamine-induced hepatocarcinogenesis. See Akiel et al., Cancer Res. 2017; 77(15):4014-25; Darr et al. Oncogene. 2014; 33(23):3024-32. On the other hand, IGFBP7 was shown to be upregulated in blood vessels of cancer tissues and was capable of promoting vascular angiogenesis (48, 64). See Komiya et al. Cancer Med. 2014; 3(3):537-49; Pen et al. Oncogene. 2008; 27(54):6834-44. IGFBP7 can be strongly induced by VEGF in vascular EC, and a synergistic effect between IGFBP7 and VEGF in angiogenesis has been reported. See Komiya et al. Cancer Med. 2014; 3(3):537-49; Hooper et al. Circ Res. 2009; 105(2):201-8. Each reference listed above is incorporated by reference in its entirety for all purposes.

The human IGFBP7 gene is located at 4q12 and encodes a polypeptide. One isoform of the polypeptide has 264 amino acid residues (SEQ ID NO: 111 that include a signal peptide domain (residues 1-26 of SEQ ID NO: 111), an insulin-binding domain (IB domain, residues 28-106 of SEQ ID NO: 111), a Kazal-like domain (residues 105-158 of SEQ ID NO: 111), and a Ig-like C2-type domain (residues 160-264 of SEQ ID NO: 111).

### Anti-IGFBP7 Antibody Moieties

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 33.

In some embodiments, the sdAb moiety comprises the amino acid sequence of SEQ ID NO: 33, or a variant thereof having at least about 80% (such as at least about any one of 80%, 85%, 90%, 95%, 96%, 97%, 98%, or 99%) sequence identify to SEQ ID NO: 33.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 32.

In some embodiments, the sdAb moiety comprises the amino acid sequence of SEQ ID NO: 32, or a variant thereof having at least about 80% (such as at least about any one of 80%, 85%, 90%, 95%, 96%, 97%, 98%, or 99%) sequence identify to SEQ ID NO: 32.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 46.

In some embodiments, the sdAb moiety comprises the amino acid sequence of SEQ ID NO: 46, or a variant thereof having at least about 80% (such as at least about any one of 80%, 85%, 90%, 95%, 96%, 97%, 98%, or 99%) sequence identify to SEQ ID NO: 46.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 47.

In some embodiments, the sdAb moiety comprises the amino acid sequence of SEQ ID NO: 47, or a variant thereof having at least about 80% (such as at least about any one of 80%, 85%, 90%, 95%, 96%, 97%, 98%, or 99%) sequence identify to SEQ ID NO: 47.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 48.

In some embodiments, the sdAb moiety comprises the amino acid sequence of SEQ ID NO: 48, or a variant thereof having at least about 80% (such as at least about any one of 80%, 85%, 90%, 95%, 96%, 97%, 98%, or 99%) sequence identify to SEQ ID NO: 48.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 34.

In some embodiments, the sdAb moiety comprises the amino acid sequence of SEQ ID NO: 34, or a variant thereof having at least about 80% (such as at least about any one of 80%, 85%, 90%, 95%, 96%, 97%, 98%, or 99%) sequence identify to SEQ ID NO: 34.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 49.

In some embodiments, the sdAb moiety comprises the amino acid sequence of SEQ ID NO: 49, or a variant thereof having at least about 80% (such as at least about any one of 80%, 85%, 90%, 95%, 96%, 97%, 98%, or 99%) sequence identify to SEQ ID NO: 49.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 50.

In some embodiments, the sdAb moiety comprises the amino acid sequence of SEQ ID NO: 50, or a variant thereof having at least about 80% (such as at least about any one of 80%, 85%, 90%, 95%, 96%, 97%, 98%, or 99%) sequence identify to SEQ ID NO: 50.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 51.

In some embodiments, the sdAb moiety comprises the amino acid sequence of SEQ ID NO: 51, or a variant thereof having at least about 80% (such as at least about any one of 80%, 85%, 90%, 95%, 96%, 97%, 98%, or 99%) sequence identify to SEQ ID NO: 51.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 35.

In some embodiments, the sdAb moiety comprises the amino acid sequence of SEQ ID NO: 35, or a variant thereof having at least about 80% (such as at least about any one of 80%, 85%, 90%, 95%, 96%, 97%, 98%, or 99%) sequence identify to SEQ ID NO: 35.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 36.

In some embodiments, the sdAb moiety comprises the amino acid sequence of SEQ ID NO: 36, or a variant thereof having at least about 80% (such as at least about any one of 80%, 85%, 90%, 95%, 96%, 97%, 98%, or 99%) sequence identify to SEQ ID NO: 36.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 37.

In some embodiments, the sdAb moiety comprises the amino acid sequence of SEQ ID NO: 37, or a variant thereof having at least about 80% (such as at least about any one of 80%, 85%, 90%, 95%, 96%, 97%, 98%, or 99%) sequence identify to SEQ ID NO: 37.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 38.

In some embodiments, the sdAb moiety comprises the amino acid sequence of SEQ ID NO: 38, or a variant thereof having at least about 80% (such as at least about any one of 80%, 85%, 90%, 95%, 96%, 97%, 98%, or 99%) sequence identify to SEQ ID NO: 38.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 39.

In some embodiments, the sdAb moiety comprises the amino acid sequence of SEQ ID NO: 39, or a variant thereof having at least about 80% (such as at least about any one of 80%, 85%, 90%, 95%, 96%, 97%, 98%, or 99%) sequence identify to SEQ ID NO: 39.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 40.

In some embodiments, the sdAb moiety comprises the amino acid sequence of SEQ ID NO: 40, or a variant thereof having at least about 80% (such as at least about any one of 80%, 85%, 90%, 95%, 96%, 97%, 98%, or 99%) sequence identify to SEQ ID NO: 40.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 41.

In some embodiments, the sdAb moiety comprises the amino acid sequence of SEQ ID NO: 41, or a variant thereof having at least about 80% (such as at least about any one of 80%, 85%, 90%, 95%, 96%, 97%, 98%, or 99%) sequence identify to SEQ ID NO: 41.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 42.

In some embodiments, the sdAb moiety comprises the amino acid sequence of SEQ ID NO: 42, or a variant thereof having at least about 80% (such as at least about any one of 80%, 85%, 90%, 95%, 96%, 97%, 98%, or 99%) sequence identify to SEQ ID NO: 42.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 43.

In some embodiments, the sdAb moiety comprises the amino acid sequence of SEQ ID NO: 43, or a variant thereof having at least about 80% (such as at least about any one of 80%, 85%, 90%, 95%, 96%, 97%, 98%, or 99%) sequence identify to SEQ ID NO: 43.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 44.

In some embodiments, the sdAb moiety comprises the amino acid sequence of SEQ ID NO: 44, or a variant thereof having at least about 80% (such as at least about any one of 80%, 85%, 90%, 95%, 96%, 97%, 98%, or 99%) sequence identify to SEQ ID NO: 44.

In some embodiments, the anti-IGFBP7 construct comprises an antibody moiety specifically recognizing IGFBP7, wherein the antibody moiety competes for binding of IGFBP7 with an antibody or antibody fragment comprising a single domain antibody (sdAb) moiety, wherein the sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31. In some embodiments, the antibody moiety is a single domain antibody (sdAb) moiety.

In some embodiments, the anti-IGFBP7 construct comprises an antibody moiety specifically recognizing IGFBP7, wherein the antibody moiety comprises a single domain antibody (sdAb) comprising a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-IGFBP7 construct comprises a humanized antibody moiety derived from a single domain antibody (sdAb) moiety comprising a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, the anti-IGFBP7 construct comprises a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in SEQ ID NO: 45.

In some embodiments, the sdAb moiety comprises the amino acid sequence of SEQ ID NO: 45, or a variant thereof having at least about 80% (such as at least about any one of 80%, 85%, 90%, 95%, 96%, 97%, 98%, or 99%) sequence identify to SEQ ID NO: 45.

In some embodiments, the anti-IGFBP7 construct comprises a polypeptide comprising a single domain antibody (sdAb) moiety specifically recognizing IGFBP7, wherein the sdAb moiety comprises a) a CDR1 comprising the amino acid sequence INTYL (SEQ ID NO: 52), b) a CDR2 comprising the amino acid sequence AITSGGSINYADSVKG (SEQ ID NO: 12), and c) a CDR3 comprising the amino acid sequence KAHPNPWGFDNDY (SEQ ID NO: 13). In some embodiments, the anti-IGFBP7 construct comprises a polypeptide comprising a single domain antibody (sdAb) moiety specifically recognizing IGFBP7, wherein the sdAb moiety comprises a) a CDR1 comprising the amino acid sequence AX₁NGAM (SEQ ID NO: 53), wherein X₁=N or I, b) a CDR2 comprising the amino acid sequence of, AITSGGSINYADSVKG (SEQ ID NO: 12), and c) a CDR3 comprising the amino acid sequence KAHPNPWGFDNDY (SEQ ID NO: 13).

In some embodiments, the anti-IGFBP7 sdAb moiety described above is camelid, chimeric, human, partially humanized, or fully humanized.

In some embodiments, the anti-IGFBP7 sdAb moiety is a VHH antibody.

In some embodiments, the anti-IGFBP7 construct comprises or is an anti-IGFBP7 fusion protein.

In some embodiments, the anti-IGFBP7 construct comprises or is a multispecific anti-IGFBP7 construct (such as a bispecific antibody).

In some embodiments, the anti-IGFBP7 construct comprises or is an anti-IGFBP7 immunoconjugate.

In some embodiments, the anti-IGFBP7 construct blocks the binding of IGFBP7 and CD93. In some embodiments, the CD93 is a human CD93. In some embodiments, the IGFBP7 is a human IGFBP7. In some embodiments, the binding of IGFBP7 to CD93 is at least blocked by about 10%, 20%, 30%, 40%, 50%, 60%, 70%, 80%, 90% or more after a pre-incubation of the anti-IGFBP7 antibody with IGFBP7 or IGFBP7-expressing cells. In some embodiments, the dose of anti-IGFBP7 antibody and IGFBP7 is at a ratio of about 1:10, 1:6, 1:3, 1:1.5, 1:1, 4:3, 2:1, or 5:1. In some embodiments, the binding of IGFBP7 to CD93 is at least blocked by about 10%, 20%, 30%, 40%, 50%, 60%, 70%, 80%, 90% or more after a pre-incubation of the anti-IGFBP7 antibody at a concentration of about 50 μg/ml, 25 μg/ml, 10 μg/ml, 5 μg/ml, 2 μg/ml, 1 μg/ml, 0.8 μg/ml, 0.6 μg/ml, or 0.4 μg/ml.

In some embodiments, the anti-IGFBP7 construct blocks the binding of IGFBP7 and MMRN2 to CD93. In some embodiments, the MMRN2 is a human MMRN2. In some embodiments, the IGFBP7 is human IGFBP7. In some embodiments, the binding of IGFBP7 to MMRN2 is at least blocked by 10%, 20%, 30%, 40%, 50%, 60%, 70%, 80%, 90% or more after a pre-incubation of the anti-IGFBP7 antibody with IGFBP7 or IGFBP7-expressing cells. In some embodiments, the anti-IGFBP7 construct does not block the binding of IGFBP7 and MMRN2.

In some embodiments, the IGFBP7 is a human IGFBP7. In some embodiments, the IGFBP7 is a mouse IGFBP7. In some embodiments, the IGFBP7 is a cynomolgus IGFBP7.

In some embodiments, the anti-IGFBP7 antibody moiety binds to both human IGFBP7 and cynomolgus IGFBP7. In some embodiments, the anti-IGFBP7 antibody moiety binds to both human IGFBP7 and mouse IGFBP7. In some embodiments, the anti-IGFBP7 antibody moiety does not bind to cynomolgus IGFBP7 and/or mouse IGFBP7.

### Anti-IGFBP7 Constructs Comprising at Least Two Antibody Moieties Specifically Recognizing IGFBP7

The present application also provides anti-IGFBP7 constructs that comprise two or more antibody moieties, wherein at least two of the two or more antibody moieties specifically recognize IGFBP7.

In some embodiments, the at least two antibody moieties are distinct. In some embodiments, the at least two antibody moieties bind to two different epitopes of IGFBP7. In some embodiments, the at least two antibody moieties bind to the same IGFBP7 epitope.

In some embodiments, the anti-IGFBP7 construct comprises a tetrameric fusion protein comprising at least two different anti-IGFBP7 antibody moieties (e.g., two different VHH domains, e.g., A1 and D4). In some embodiments, the two different anti-IGFBP7 antibody moieties bind to two distinct epitopes of IGFBP7. In some embodiments, the fusion protein comprises an Fc fragment.

In some embodiments, the at least two antibody moieties are the same.

In some embodiments, the at least two antibody moieties are both single domain antibody moieties (such as any of the sdAb moieties described herein).

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs, wherein the second sdAb moiety comprises a CDR1 comprising the amino acid sequence of SEQ ID NO: 2, a CDR2 comprising the amino acid sequence of SEQ ID NO: 3, and a CDR3 comprising the amino acid sequence of SEQ ID NO: 4, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 2, a CDR2 comprising the amino acid sequence of SEQ ID NO: 3, and a CDR3 comprising the amino acid sequence of SEQ ID NO: 4.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; wherein the second sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19; wherein the second sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; wherein the second sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25; wherein the second sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs. In some embodiments, there is provided an anti-IGFBP7 antibody construct comprising a) a first anti-IGFBP7 antibody moiety comprising a first single domain antibody (sdAb) moiety and b) a second anti-IGFBP7 antibody moiety comprising a second sdAb moiety, wherein the first sdAb moiety comprises a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28; wherein the second sdAb moiety comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31.

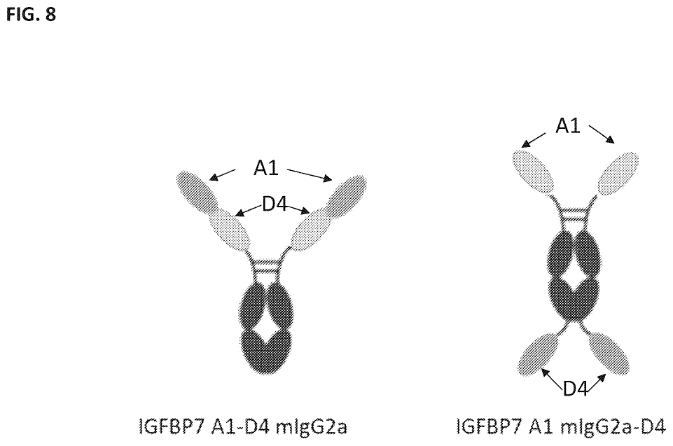

The two antibody moieties (e.g., two sdAbs) described herein can be fused together in any ways that would allow the proper properties of both moieties (e.g., binding to IGFBP7). In some embodiments, the first anti-IGFBP7 antibody moiety is fused to (e.g., directly fused to) N-terminus of the second anti-IGFBP7 antibody moiety. In some embodiments, the first anti-IGFBP7 antibody moiety is fused to (e.g., directly fused to) C-terminus of the second anti-IGFBP7 antibody moiety. In some embodiments, the construct comprises a Fc fragment comprising a first Fc domain and a second Fc domain, wherein each Fc domain comprises a CH2 domain and a CH3 domain. In some embodiments, both antibody moieties are fused to N-terminus of the two Fc domains. See for example FIG. 8. In some embodiments, both antibody moieties are fused to C-terminus of the two Fc domains. In some embodiments, the first anti-IGFBP7 antibody moiety is fused to N-terminus of both Fc domains, and the second anti-IGFBP7 antibody moiety is fused to C-terminus of both Fc domains. In some embodiments, the first anti-IGFBP7 antibody moiety is fused to C-terminus of both Fc domains, and the second anti-IGFBP7 antibody moiety is fused to N-terminus of both Fc domains. In some embodiments, the first or second antibody moiety is fused to the C-terminus of one or both of the Fc domains via a linker (e.g., a GS linker). In some embodiments, the first or second antibody moiety is fused to the N-terminus of one or both of the Fc domains cia a linker (e.g., a GS linker). In some embodiments, the Fc domain comprises a hinge region. In some embodiments, the Fc domain has a mutation that results in a reduced effector function.

In some embodiments, the construct comprises a first polypeptide and a second polypeptide, wherein both polypeptide each comprises a) a first single domain antibody (sdAb) moiety, b) a second sdAb moiety and c) a Fc domain, wherein two Fc domains form a Fc fragment, wherein the first sdAb comprises i) a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, wherein the first sdAb is directly fused via its C-terminus to the N-terminus of the second sdAb, and wherein the second sdAb is fused via its C-terminus to the N-terminus of both Fc domains, optionally via a linker (e.g., a GS linker).

In some embodiments, the construct comprises a first polypeptide and a second polypeptide, wherein both polypeptide each comprises a) a first single domain antibody (sdAb) moiety, b) a second sdAb moiety and c) a Fc domain, wherein two Fc domains form a Fc fragment, wherein the first sdAb comprises i) a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, wherein the second sdAb moiety comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, wherein the first sdAb is fused via its C-terminus to N-terminus of both Fc domains, and wherein the second sdAb is fused via its N-terminus to C-terminus of both Fc domains optionally via a linker (e.g., a GS linker).

In some embodiments, the first or second anti-IGFBP7 antibody moiety is a humanized antibody moiety derived from any of the antibody moieties described in this section.

In some embodiments, the first or second anti-IGFBP7 antibody moiety is an antibody moiety that competes for a binding epitope of IGFBP7 with any of the antibody moieties described in this section.

### Multispecific Anti-IGFBP7 Constructs Binding to a Second Agent

The anti-IGFBP7 constructs in some embodiments comprise a multispecific (e.g., bispecific) anti-IGFBP7 construct comprising an anti-IGFBP7 antibody moiety (e.g., any one of the anti-IGFBP7 antibody moieties described herein), and a second binding moiety (such as a second antibody moiety) specifically recognizes a second antigen that is different from IGFBP7. In some embodiments, the second antigen is an immune checkpoint molecule. In some embodiments, the second antigen is PD-1 or PD-L1. In some embodiments, the second antigen is a tumor antigen. In some embodiments, the second antigen is an angiogenic agent. In some embodiments, the angiogenic agent is VEGF (e.g., a human VEGF). In some embodiments, the angiogenic agent is a VEGF receptor. In some embodiments, the angiogenic agent is a VEGFR1 (e.g., a human VEGFR1) or fragment thereof. In some embodiments, the angiogenic agent is a VEGFR2 (e.g., a human VEGFR2) or fragment thereof.

In some embodiments, the second binding moiety is fused to the anti-IGFBP7 antibody moiety via a linker (such as any of the linkers described herein).

In some embodiments, the second antibody moiety is a full-length antibody, a Fab, a Fab′, a (Fab′)₂, an Fv, a single chain Fv (scFv), a scFv-scFv, a minibody, a diabody, a nanobody, or a sdAb.

In some embodiments, the anti-IGFBP7 construct is a multispecific (e.g., bispecific) anti-IGFBP7 construct comprising a) an anti-IGFBP7 antibody moiety according to any one of the anti-IGFBP7 antibody moieties described herein; b) a second antibody moiety specifically recognizing PD-L1 (an anti-PD-L1 antibody moiety such as any of those described herein) or PD-1 (an anti-PD-1 antibody moiety such as any of those described herein).

In some embodiments, the anti-IGFBP7 construct is a multispecific (e.g., bispecific) anti-IGFBP7 construct comprising a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the anti-IGFBP7 antibody moiety described herein) fused to at least one or both of the heavy chains of the anti-PD-L1 or anti-PD-1 full-length antibody. In some embodiments, the anti-IGFBP7 antibody moiety is fused to N-terminus of both heavy chains. In some embodiments, the anti-IGFBP7 antibody moiety is fused to C-terminus of both heavy chains

In some embodiments, the anti-IGFBP7 construct is a multispecific (e.g., bispecific) anti-IGFBP7 construct comprising a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to at least one or both of the light chains of the anti-PD-L1 or anti-PD-1 full-length antibody. In some embodiments, the anti-IGFBP7 antibody moiety is fused to N-terminus of both light chains. In some embodiments, the anti-IGFBP7 antibody moiety is fused to C-terminus of both light chains.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs.

In some embodiments, the anti-IGFBP7 construct comprises a) an anti-PD-L1 or anti-PD-1 antibody moiety comprising a full-length antibody comprising two heavy chains and two light chains, wherein the two heavy chains each comprises a heavy chain variable region (V_{H}) and the two light chains each comprises a light chain variable region (V_{L}), b) an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs.

In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, there is provided an anti-IGFBP7 construct comprising a) an anti-IGFBP7 antibody moiety and b) a binding moiety that specifically recognizes a VEGF (e.g., a human VEGF) or a VEGFR (e.g., a human VEGFR1, e.g., a human VEGFR2). In some embodiments, the binding moiety that specifically recognizes a VEGF or a VEGFR comprises the amino acid sequence of SEQ ID NO: 98. In some embodiments, anti-IGFBP7 sdAb comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the anti-IGFBP7 sdAb is fused to the binding moiety that specifically recognizes a VEGF or VEGFR via a linker or a constant region of an immunoglobulin (such as a human IgG).

In some embodiments, there is provided an anti-IGFBP7 construct comprising a) an anti-IGFBP7 antibody moiety and b) a binding moiety that specifically recognizes a VEGF (e.g., a human VEGF) or a VEGFR (e.g., a human VEGFR1, e.g., a human VEGFR2). In some embodiments, the binding moiety that specifically recognizes a VEGF or a VEGFR comprises the amino acid sequence of SEQ ID NO: 98. In some embodiments, anti-IGFBP7 sdAb comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, the anti-IGFBP7 sdAb is fused to the binding moiety that specifically recognizes a VEGF or VEGFR via a linker or a constant region of an immunoglobulin (such as a human IgG).

In some embodiments, there is provided an anti-IGFBP7 construct comprising a) an anti-VEGF (e.g., an anti-human VEGF) or anti-VEGFR (e.g., an anti-human VEGFR1, e.g., an anti-human VEGFR2) full-length antibody comprising two heavy chains and two light chains, and b) an anti-IGFBP7 single domain antibody (sdAb), wherein the anti-IGFBP7 sdAb is fused to one or both of the heavy chains and/or light chains of the full-length antibody. In some embodiments, the anti-IGFBP7 sdAb is fused to C-terminus of both heavy chains of the full-length antibody. In some embodiments, the anti-IGFBP7 sdAb is fused to N-terminus of both heavy chains of the full-length antibody. In some embodiments, the anti-IGFBP7 sdAb is fused to C-terminus of both light chains of the full-length antibody. In some embodiments, the anti-IGFBP7 sdAb is fused to N-terminus of both light chains of the full-length antibody. In some embodiments, the anti-IGFBP7 sdAb comprises a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the anti-IGFBP7 sdAb comprises a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, the full-length antibody specifically recognizes VEGF, comprising a heavy chain variable region (V_{H}) and a light chain variable region (V_{L}), wherein the V_{H} comprises the HC-CDR1 comprising the amino acid sequence of SEQ ID NO: 99, the HC-CDR2 comprising the amino acid sequence of SEQ ID NO: 100, and the HC-CDR3 comprising the amino acid sequence of SEQ ID NO: 101, and the V_{L} comprises the LC-CDR1 comprising the amino acid sequence of SEQ ID NO: 102, the LC-CDR2 comprising the amino acid sequence of SEQ ID NO: 103, and the LC-CDR3 comprising the amino acid sequence of SEQ ID NO: 104. In some embodiments, the full-length antibody specifically recognizes VEGFR2, comprising a heavy chain variable region (V_{H}) and a light chain variable region (V_{L}), wherein the V_{H} comprises the HC-CDR1 comprising the amino acid sequence of SEQ ID NO: 105, the HC-CDR2 comprising the amino acid sequence of SEQ ID NO: 106, and the HC-CDR3 comprising the amino acid sequence of SEQ ID NO: 107, and the V_{L} comprises the LC-CDR1 comprising the amino acid sequence of SEQ ID NO: 108, the LC-CDR2 comprising the amino acid sequence of SEQ ID NO: 109, and the LC-CDR3 comprising the amino acid sequence of SEQ ID NO: 110. In some embodiments, the anti-IGFBP7 sdAb is fused to the full-length antibody via a linker (such as any of the linkers described above).

In some embodiments, the second binding (e.g., the second antibody moiety) and the anti-IGFBP7 antibody moiety are fused with each other via a linker such as any of the linkers described herein with any operable form that allows the proper function of the binding moieties. In some embodiments, the linker is a GS linker. In some embodiments, the linker is selected from the group consisting of SEQ ID NOs: 54-61.

### Exemplary Anti-PD-L1 Antibody Moieties

Exemplary anti-PD-L1 antibody moieties include, but not are limited to those described in WO2019228514A1, WO2019227490A1 and WO2020019232A1.

In some embodiments, the anti-PD-L1 antibody moiety (such as an scFv) used in multispecific anti-IGFBP7 constructs comprises an antibody moiety comprising a heavy chain variable region (V_{H}) and a light chain variable region (V_{L}), wherein the antibody moiety competes for a binding epitope of PD-L1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 62, the HC-CDR2 comprising the amino acid sequence of SEQ ID NO: 63, and the HC-CDR3 comprising the amino acid sequence of SEQ ID NO: 64, and the V_{L-2} comprises the LC-CDR1 comprising the amino acid sequence of SEQ ID NO: 65, the LC-CDR2 comprising the amino acid sequence of SEQ ID NO: 66, and the LC-CDR3 comprising the amino acid sequence of SEQ ID NO: 67.

In some embodiments, the anti-PD-L1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 68, 69, or 70; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 71, 72, or 73.

In some embodiments, the anti-PD-L1 antibody moiety (such as an scFv) used in multispecific anti-IGFBP7 constructs comprises a heavy chain variable region (V_{H}) and a light chain variable region (V_{L}), wherein: a) the V_{H} comprises an HC-CDR1 comprising the amino acid sequence of SEQ ID NO: 62, an HC-CDR2 comprising the amino acid sequence of SEQ ID NO: 63, and an HC-CDR3 comprising the amino acid sequence of SEQ ID NO: 64, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 65, an LC-CDR2 comprising the amino acid sequence of any one of SEQ ID NO: 66, and an LC-CDR3 comprising the amino acid sequence of any one of SEQ ID NO: 67, or a variant thereof comprising up to a total of about 5, 4, 3, 2, or 1 amino acid substitutions in the LC-CDRs.

In some embodiments, the V_{H} comprises an amino acid sequence of SEQ ID NO: 68, 69, or 70, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 71, 72, or 73, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 68, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 71, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 69, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 72, or a variant comprising an amino acid sequence having at least about 80% (such as at least about any one of 80%, 85%, 90%, 95%, 96%, 97%, 98%, or 99%) sequence identity. In some embodiments, the V_{H} comprises an amino acid sequence of SEQ ID NO: 70, or a variant comprising an amino acid sequence having at least about 80% (such as at least about any one of 80%, 85%, 90%, 95%, 96%, 97%, 98%, or 99%) sequence identity; and the V_{L} comprises an amino acid sequence of SEQ ID NO: 73, or a variant comprising an amino acid sequence having at least about 80% (such as at least about any one of 80%, 85%, 90%, 95%, 96%, 97%, 98%, or 99%) sequence identity.

### Exemplary Anti-PD-1 Antibody Moieties

Exemplary anti-PD-1 antibody moieties include, but not are limited to those described in WO2018133842 and WO2018133837.

In some embodiments, the anti-PD-1 antibody moiety (such as an scFv) used in multispecific anti-IGFBP7 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 74, the HC-CDR2 comprising the amino acid sequence of SEQ ID NO: 75, and the HC-CDR3 comprising the amino acid sequence of SEQ ID NO: 76, and the V_{L-2} Comprises the LC-CDR1 comprising the amino acid sequence of SEQ ID NO: 77, the LC-CDR2 comprising the amino acid sequence of SEQ ID NO: 78, and the LC-CDR3 comprising the amino acid sequence of SEQ ID NO: 79.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 92; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 93.

In some embodiments, the anti-PD-1 antibody moiety (such as an scFv) used in multispecific anti-IGFBP7 constructs comprises a heavy chain variable region (V_{H}) and a light chain variable region (V_{L}), wherein: a) the V_{H} comprises an HC-CDR1 comprising the amino acid sequence of SEQ ID NO: 74, an HC-CDR2 comprising the amino acid sequence of SEQ ID NO: 75, and an HC-CDR3 comprising the amino acid sequence of SEQ ID NO: 76, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 77, an LC-CDR2 comprising the amino acid sequence of any one of SEQ ID NO: 78, and an LC-CDR3 comprising the amino acid sequence of any one of SEQ ID NO: 79, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the second antibody moiety comprises a humanized antibody moiety derived from a murine antibody comprising a heavy chain variable region (V_{H}) comprising the amino acid sequence set forth in SEQ ID NO: 92 and a light chain variable region (V_{L}) comprising the amino acid sequence forth in SEQ ID NO: 93.

In some embodiments, the anti-PD-1 antibody moiety (such as an scFv) used in multispecific anti-IGFBP7 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 80, the HC-CDR2 comprising the amino acid sequence of SEQ ID NO: 81, and the HC-CDR3 comprising the amino acid sequence of SEQ ID NO: 82, and the V_{L-2} Comprises the LC-CDR1 comprising the amino acid sequence of SEQ ID NO: 83, the LC-CDR2 comprising the amino acid sequence of SEQ ID NO: 84, and the LC-CDR3 comprising the amino acid sequence of SEQ ID NO: 85.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 94; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 95.

In some embodiments, the anti-PD-1 antibody moiety (such as an scFv) used in multispecific anti-IGFBP7 constructs comprises a heavy chain variable region (V_{H}) and a light chain variable region (V_{L}), wherein: a) the V_{H} comprises an HC-CDR1 comprising the amino acid sequence of SEQ ID NO: 80, an HC-CDR2 comprising the amino acid sequence of SEQ ID NO: 81, and an HC-CDR3 comprising the amino acid sequence of SEQ ID NO: 82, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 83, an LC-CDR2 comprising the amino acid sequence of any one of SEQ ID NO: 84, and an LC-CDR3 comprising the amino acid sequence of any one of SEQ ID NO: 85, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the anti-PD-1 antibody moiety comprises a humanized antibody moiety derived from a murine antibody comprising a heavy chain variable region (V_{H}) comprising the amino acid sequence set forth in SEQ ID NO: 94 and a light chain variable region (V_{L}) comprising the amino acid sequence forth in SEQ ID NO: 95.

In some embodiments, the anti-PD-1 antibody moiety (such as an scFv) used in multispecific anti-IGFBP7 constructs comprises an antibody moiety comprising a heavy chain variable region (V_{H}) and a light chain variable region (V_{L}), wherein the antibody moiety competes for a binding epitope of PD-1 with an antibody or antibody fragment comprising a second heavy variable region (V_{H-2}) and a second light chain variable region (V_{L-2}), wherein the V_{H-2} comprises the HC-CDR1 comprising the amino acid sequence of SEQ ID NO: 86, the HC-CDR2 comprising the amino acid sequence of SEQ ID NO: 87, and the HC-CDR3 comprising the amino acid sequence of SEQ ID NO: 88, and the V_{L-2} Comprises the LC-CDR1 comprising the amino acid sequence of SEQ ID NO: 89, the LC-CDR2 comprising the amino acid sequence of SEQ ID NO: 90, and the LC-CDR3 comprising the amino acid sequence of SEQ ID NO: 91.

In some embodiments, the anti-PD-1 moiety comprises a HC-CDR1, a HC-CDR2, and a HC-CDR3, respectively comprising the amino acid sequences of a CDR1, a CDR2, and a CDR3 within a V_{H} chain region having the sequence set forth in SEQ ID NO: 96; and a LC-CDR1, a LC-CDR2, and a LC-CDR3, respectively comprising the amino acid sequences of a CDR1, a CDR2, and a CDR3 within a V_{L} chain region having the sequence set forth in SEQ ID NO: 97.

In some embodiments, the anti-PD-1 antibody moiety (such as an scFv) used in multispecific anti-IGFBP7 constructs comprises a heavy chain variable region (V_{H}) and a light chain variable region (V_{L}), wherein: a) the V_{H} comprises an HC-CDR1 comprising the amino acid sequence of SEQ ID NO: 86, an HC-CDR2 comprising the amino acid sequence of SEQ ID NO: 87, and an HC-CDR3 comprising the amino acid sequence of SEQ ID NO: 88, or a variant thereof comprising up to a total of about 5, 4, 3, 2, or 1 amino acid substitutions in the HC-CDRs; and b) the V_{L} comprises an LC-CDR1 comprising the amino acid sequence of SEQ ID NO: 89, an LC-CDR2 comprising the amino acid sequence of any one of SEQ ID NO: 90, and an LC-CDR3 comprising the amino acid sequence of any one of SEQ ID NO: 91, or a variant thereof comprising up to a total of about 5, 4, 3, 2, or 1 amino acid substitutions in the LC-CDRs. In some embodiments, the amino acid substitutions described above are limited to “exemplary substitutions” shown in Table 2 of this application. In some embodiments, the amino acid substitutions are limited to “preferred substitutions” shown in Table 2 of this application.

In some embodiments, the second antibody moiety comprises a humanized antibody moiety derived from a murine antibody comprising a heavy chain variable region (V_{H}) comprising the amino acid sequence set forth in SEQ ID NO: 96 and a light chain variable region (V_{L}) comprising the amino acid sequence forth in SEQ ID NO: 97.

### Exemplary Binding Moieties Specifically Recognizing VEGF

Exemplary binding moieties specifically recognizing VEGF include, but not are limited to avastin, ramucirumab, or VEGF-trap (Aflibercept), or a variant or a functional portion thereof.

In some embodiments, the binding moiety that specifically recognizes VEGF used in multispecific anti-IGFBP7 constructs is an antibody moiety (such as an scFv) comprising an antibody moiety comprising a heavy chain variable region (V_{H}) and a light chain variable region (V_{L}), wherein the V_{H} comprises the HC-CDR1 comprising the amino acid sequence of SEQ ID NO: 99, the HC-CDR2 comprising the amino acid sequence of SEQ ID NO: 100, and the HC-CDR3 comprising the amino acid sequence of SEQ ID NO: 101, and the V_{L} comprises the LC-CDR1 comprising the amino acid sequence of SEQ ID NO: 102, the LC-CDR2 comprising the amino acid sequence of SEQ ID NO: 103, and the LC-CDR3 comprising the amino acid sequence of SEQ ID NO: 104.

In some embodiments, the binding moiety that specifically recognizes VEGF used in multispecific anti-IGFBP7 constructs is an antibody moiety (such as an scFv) comprising an antibody moiety comprising a heavy chain variable region (V_{H}) and a light chain variable region (V_{L}), wherein the V_{H} comprises the HC-CDR1 comprising the amino acid sequence of SEQ ID NO: 105, the HC-CDR2 comprising the amino acid sequence of SEQ ID NO: 106, and the HC-CDR3 comprising the amino acid sequence of SEQ ID NO: 107, and the V_{L} comprises the LC-CDR1 comprising the amino acid sequence of SEQ ID NO: 108, the LC-CDR2 comprising the amino acid sequence of SEQ ID NO: 109, and the LC-CDR3 comprising the amino acid sequence of SEQ ID NO: 110.

In some embodiments, the binding moiety that specifically recognizes VEGF used in multispecific anti-IGFBP7 constructs comprises the amino acid sequence of SEQ ID NO: 99.

### Anti-IGFBP7 Fusion Proteins

The anti-IGFBP7 constructs in some embodiments comprise an anti-IGFBP7 antibody moiety (e.g., an anti-IGFBP7 sdAb moiety) and a second moiety.

In some embodiments, the second moiety is a ligand (e.g., a ligand that interacts with another molecule). In some embodiments, the second moiety is a peptide. In some embodiments, the second moiety is a cytokine.

In some embodiments, the second moiety comprises a half-life extending moiety.

In some embodiments, the half-life extending moiety is an albumin binding moiety (e.g., an albumin binding antibody moiety). In some embodiments, the anti-IGFBP7 antibody moiety and the half-life extending moiety is linked via a linker (such as any of the linkers described in the “Linkers” section).

In some embodiments, the half-life extending moiety is an Fc fragment. In some embodiments, the Fc fragment is selected from the group consisting of Fc fragments from IgG, IgA, IgD, IgE, IgM, and combinations and hybrids thereof. In some embodiments, the Fc fragment is selected from the group consisting of Fc fragments from IgG1, IgG2, IgG3, IgG4, and combinations and hybrids thereof. In some embodiments, the Fc has one or more amino acid modifications that result in extended half-life of the antibody moiety in the serum. In some embodiments, the Fc fragment has a reduced effector function as compared to the corresponding wildtype Fc fragment. In some embodiments, the Fc fragment has an enhanced effector function as compared to the corresponding wildtype Fc fragment.

In some embodiments, the Fc fragment is derived from an IgG2a (such as a mouse IgG2a).

In some embodiments, the second moiety is a lipid. In some embodiments, the lipid is conjugated to the anti-IGFBP7 antibody moiety (such as an anti-IGFBP7 antibody), and can bind to albumin, thereby extending the half-life of the anti-IGFBP7 antibody moiety.

In some embodiments, the second moiety is an albumin or a portion of albumin (e.g., human albumin, e.g., human serum albumin).

### Anti-IGFBP7 Immunoconjugates

The present application also provides anti-IGFBP7 immunoconjugates comprising an anti-IGFBP7 antibody moiety (such as any of the IGFBP7 antibody moieties described herein) and a second agent. In some embodiments, the second agent is a therapeutic agent. In some embodiments, the second agent is a label.

In some embodiments, the second agent is a cytotoxic agent. In some embodiments, the cytotoxic agent is a chemotherapeutic agent. In some embodiments, the cytotoxic agent is a growth inhibitory agent. In some embodiments, the cytotoxic agent is a toxin (e.g., protein toxins, enzymatically active toxins of bacterial, fungal, plant, or animal origin, or fragments thereof). In some embodiments, the cytotoxic agent is a radioactive isotype (i.e., a radioconjugate).

Immunoconjugates allow for the targeted delivery of a drug moiety to a tumor, and, in some embodiments intracellular accumulation therein, where systemic administration of unconjugated drugs may result in unacceptable levels of toxicity to normal cells (Polakis P. (2005) Current Opinion in Pharmacology 5:382-387).

Production of immunoconjugates described herein can be found in, for example, U.S. Pat. Nos. 9,562,099 and 7,541,034, which are hereby incorporated by references in their entirety.

### Linkers

In some embodiments, the anti-IGFBP7 constructs described herein comprise one or more linkers between two moieties (e.g., the anti-IGFBP7 antibody moiety and the half-life extending moiety, the anti-IGFBP7 antibody moiety and the second binding moiety in the multispecific constructs described above). The length, the degree of flexibility and/or other properties of the linker(s) used in the anti-IGFBP7 constructs may have some influence on properties, including but not limited to the affinity, specificity or avidity for one or more particular antigens or epitopes. For example, longer linkers may be selected to ensure that two adjacent domains do not sterically interfere with one another. In some embodiment, a linker (such as peptide linker) comprises flexible residues (such as glycine and serine) so that the adjacent domains are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker.

Other linker considerations include the effect on physical or pharmacokinetic properties of the resulting compound, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

### Peptide Linkers

The peptide linker may have a naturally occurring sequence, or a non-naturally occurring sequence. For example, a sequence derived from the hinge region of heavy chain only antibodies may be used as the linker. See, for example, WO1996/34103.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acid to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). A particularly preferred amino acid in context of the “peptide linker” is Gly. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The peptide linker can be a stable linker, which is not cleavable by proteases, especially by Matrix metalloproteinases (MMPs).

The linker can also be a flexible linker. Exemplary flexible linkers include glycine polymers (G)ₙ(SEQ ID NO: 54), glycine-serine polymers (including, for example, (GS). (SEQ ID NO: 55), (GSGGS)ₙ (SEQ ID NO: 56), (GGGGS)ₙ (SEQ ID NO: 57), and (GGGS)ₙ (SEQ ID NO: 58), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (See Scheraga, Rev. Computational Chem. 11 173-142 (1992)). The ordinarily skilled artisan will recognize that design of an antibody fusion protein can include linkers that are all or partially flexible, such that the linker can include a flexible linker portion as well as one or more portions that confer less flexible structure to provide a desired antibody fusion protein structure.

Furthermore, exemplary linkers also include the amino acid sequence of such as (GGGGS)ₙ (SEQ ID NO: 57), wherein n is an integer between 1 and 8, e.g. (GGGGS)₃ (SEQ ID NO: 59; hereinafter referred to as “(G4S)3” or “GS3”), or (GGGGS)₆ (SEQ ID NO: 60; hereinafter referred to as “(G4S)6” or “GS6”). In some embodiments, the peptide linker comprises the amino acid sequence of (GSTSGSGKPGSGEGS)ₙ (SEQ ID NO: 61), wherein n is an integer between 1 and 3.

### Non-Peptide Linkers

Coupling of two moieties may be accomplished by any chemical reaction that will bind the two molecules so long as both components retain their respective activities, e.g., binding to IGFBP7 and a second agent in an anti-IGFBP7 multispecific antibody, respectively. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents may be useful in coupling protein molecules in this context. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (See Killen and Lindstrom, Jour. Immun. 133:1335-2549 (1984); Jansen et al., Immunological Reviews 62:185-216 (1982); and Vitetta et al., Science 238:1098 (1987)).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido] hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus may lead to bispecific antibodies with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form antibody fusion protein with increased stability. Disulfide linkages, are in general, less stable than other linkages because the disulfide linkage is cleaved in vitro, resulting in less antibody fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

### Anti-IGFBP7 Antibody Moiety or Construct Variants

a) Antibody Affinity

Binding specificity of the anti-IGFBP7 antibody moieties can be determined experimentally by methods known in the art. Such methods comprise, but are not limited to Western blots, ELISA-, RIA-, ECL-, IRMA-, EIA-, BLI, SPR, BIACORE™-tests and peptide scans.

In some embodiments, the K_{D} of the binding between the antibody moiety and IGFBP7 is about 10⁻⁷ M to about 10⁻¹² M, about 10⁻⁷ M to about 10⁻⁸ M, about 10⁻⁸ M to about 10⁻⁹ M, about 10⁻⁹ M to about 10⁻¹⁰ M, about 10⁻¹⁰ M to about 10⁻¹¹ M, about 10⁻¹¹ M to about 10⁻¹² M, about 10⁻⁷ M to about 10⁻¹² M, about 10⁻⁸ M to about 10⁻¹² M, about 10⁻⁹ M to about 10⁻¹² M, about 10⁻¹⁰ M to about 10⁻¹² M, about 10⁻⁷ M to about 10⁻¹¹ M, about 10⁻⁸ M to about 10⁻¹¹ M, about 10⁻⁹ M to about 10⁻¹¹ M, about 10⁻⁷ M to about 10⁻¹⁰ M, about 10⁻⁸ M to about 10⁻¹⁰ M, or about 10⁻⁷ M to about 10⁻⁹ M. In some embodiments, the K_{D} of the binding between the antibody moiety and IGFBP7 is stronger than about any one of 10⁻⁷ M, 10⁻⁸ M, 10⁻⁹ M, 10⁻¹⁰ M, 10⁻¹¹ M, or 10⁻¹² M. In some embodiments, the IGFBP7 is a human IGFBP7.

In some embodiments, the Kₒₙ of the binding between the antibody moiety and IGFBP7 is about 10³ M⁻¹s⁻¹ to about 10⁸ M⁻¹s⁻¹, about 10³ M⁻¹s⁻¹ to about 10⁴ M⁻¹s⁻¹, about 10⁴ M⁻¹s⁻¹ to about 10⁵ M⁻¹s⁻¹, about 10⁵ M⁻¹s⁻¹ to about 10⁶ M⁻¹s⁻¹, about 10⁶ M⁻¹s⁻¹ to about 10⁷ M⁻¹s⁻¹, or about 10⁷ M⁻¹s⁻¹ to about 10⁸ M⁻¹s⁻¹. In some embodiments, the Kₒₙ of the binding between the antibody moiety and IGFBP7 is about 10³ M⁻¹s⁻¹ to about 10⁵ M⁻¹s⁻¹, about 10⁴ M^{-s1}s⁻¹ to about 10⁶ M⁻¹s⁻¹, about 10⁵ M⁻¹s⁻¹ to about 10⁷ M⁻¹s⁻¹, about 10⁶ M⁻¹s⁻¹ to about 10⁸ M⁻¹s⁻¹, about 10⁴ M⁻¹s⁻¹ to about 10⁷ M⁻¹s⁻¹, or about 10⁵ M⁻¹s⁻¹ to about 10⁸ M⁻¹s⁻¹. In some embodiments, the Kₒₙ of the binding between the antibody moiety and IGFBP7 is no more than about any one of 10³ M⁻¹s⁻¹, 10⁴ M⁻¹s⁻¹, 10⁵ M⁻¹s⁻¹, 10⁶ M⁻¹s⁻¹, 10⁷ M⁻¹s⁻¹ or 10⁸ M⁻¹s⁻¹. In some embodiments, IGFBP7 is human IGFBP7.

In some embodiments, the K_{off} of the binding between the antibody moiety and IGFBP7 is about 1 s⁻¹ to about 10⁻⁶ s⁻¹, about 1 s⁻¹ to about 10⁻² s⁻¹, about 10⁻² s⁻¹ to about 10⁻³ s⁻¹, about 10⁻³ s⁻¹ to about 10⁻⁴ s⁻¹, about 10⁻⁴ s⁻¹ to about 10⁻⁵ s⁻¹, about 10⁻⁵ s⁻¹ to about 10⁻⁶ s⁻¹, about 1 s⁻¹ to about 10⁻⁵ s⁻¹, about 10⁻² s⁻¹ to about 10⁻⁶ s⁻¹, about 10⁻³ s⁻¹ to about 10⁻⁶ s⁻¹, about 10⁻⁴ s⁻¹ to about 10⁻⁶ s⁻¹, about 10⁻² s⁻¹ to about 10⁻⁵ s⁻¹, or about 10⁻³ s⁻¹ to about 10⁻⁵ s⁻¹.

In some embodiments, the K_{off} of the binding between the antibody moiety and IGFBP7 is at least about any one of 1 s⁻¹, 10⁻² s⁻¹, 10⁻³ s⁻¹, 10⁻⁴ s⁻¹, 10⁻⁵ s⁻¹ or 10⁻⁶ s⁻¹. In some embodiments, IGFBP7 is human IGFBP7.

In some embodiments, the binding affinity of the anti-IGFBP7 antibody moiety or anti-IGFBP7 construct are higher (for example, has a smaller K_{D} value) than an existing anti-IGFBP7 antibody (e.g., anti-human IGFBP7 antibody, e.g., MM01).

b) Chimeric or Humanized Antibodies

In some embodiments, the anti-IGFBP7 antibody moiety is a chimeric antibody. Certain chimeric antibodies are described, e.g., in U.S. Pat. No. 4,816,567; and Morrison et al., Proc. Natl. Acad. Sci. USA, 81:6851-6855 (1984)). In some embodiments, a chimeric antibody comprises a non-human variable region (e.g., a variable region derived from llama) and a human constant region. In some embodiments, a chimeric antibody is a “class switched” antibody in which the class or subclass has been changed from that of the parent antibody. Chimeric antibodies include antigen-binding fragments thereof.

In some embodiments, the anti-IGFBP7 antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); Framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

It is understood that the humanization of non-human derived antibodies is a common and routinely used art. It is therefore understood that a humanized format of any and all of the anti-IGFBP7 antibodies disclosed in Sequence Table can be used in a preclinical or clinical setting. In cases where a humanized format of any of the referenced anti-IGFBP7 antibodies or their antigen-binding regions thereof is used in such a preclinical or clinical setting, the then humanized format is expected to bear the same or similar biological activities and profiles as the original non-humanized format.

c) Human Antibodies

In some embodiments, the anti-IGFBP7 antibody moiety is a human antibody (known as human domain antibody, or human DAb). Human antibodies can be produced using various techniques known in the art. Human antibodies are described generally in van Dijk and van de Winkel, Curr. Opin. Pharmacol. 5: 368-74 (2001), Lonberg, Curr. Opin. Immunol. 20:450-459 (2008), and Chen, Mol. Immunol. 47(4):912-21 (2010). Transgenic mice or rats capable of producing fully human single-domain antibodies (or DAb) are known in the art. See, e.g., US20090307787A1, U.S. Pat. No. 8,754,287, US20150289489A1, US20100122358A1, and WO2004049794.

Human antibodies (e.g., human DAbs) may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies (e.g., human DAbs) can also be made by hybridoma-based methods. Human myeloma and mouse-human heteromyeloma cell lines for the production of human monoclonal antibodies have been described (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies (e.g., human DAbs) may also be generated by isolating Fv clone variable domain sequences selected from human-derived phage display libraries. Such variable domain sequences may then be combined with a desired human constant domain. Techniques for selecting human antibodies from antibody libraries are described below.

d) Library-Derived Antibodies

The anti-IGFBP7 antibody moieties described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004). Methods for constructing single-domain antibody libraries have been described, for example, See U.S. Pat. No. 7,371,849.

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically displays antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

e) Substitution, Insertion, Deletion and Variants

In some embodiments, anti-IGFBP7 antibody variants having one or more amino acid substitutions are provided. Sites of interest for substitutional mutagenesis include the HVRs (or CDRs) and FRs. Conservative substitutions are shown in Table 2 under the heading of “Preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2
Amino acid substitutions

| Original | | |
|---|---|---|
| Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity, improved solubility) relative to the parent antibody and/or will have substantiality retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or CDRs.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

f) Glycosylation Variants

In some embodiments, the anti-IGFBP7 construct is altered to increase or decrease the extent to which the construct is glycosylated. Addition or deletion of glycosylation sites to an antibody may be conveniently accomplished by altering the amino acid sequence such that one or more glycosylation sites is created or removed.

Where the anti-IGFBP7 construct comprises an Fc region, the carbohydrate attached thereto may be altered. Native antibodies produced by mammalian cells typically comprise a branched, biantennary oligosaccharide that is generally attached by an N-linkage to Asn297 of the C_{H}2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the antibody moiety may be made in order to create antibody variants with certain improved properties.

In some embodiments, the anti-IGFBP7 construct has a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the anti-IGFBP7 construct has bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

g) Fc Region Variants

In some embodiments, the anti-IGFBP7 construct comprises an Fc fragment.

The term “Fc region,” “Fc domain,” “Fc fragment” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, M D, 1991.

In some embodiments, the Fc fragment is from an immunoglobulin selected from the group consisting of IgG, IgA, IgD, IgE, IgM, and combinations and hybrids thereof. In some embodiments, the Fc fragment is from an immunoglobulin selected from the group consisting of IgG1, IgG2, IgG3, IgG4, and combinations and hybrids thereof.

In some embodiments, the Fc fragment has a reduced effector function as compared to corresponding wildtype Fc fragment (such as at least about 30%, 40%, 50%, 60%, 70%, 80%, 85%, 90%, or 95% reduced effector function as measured by the level of antibody-dependent cellular cytotoxicity (ADCC)).

In some embodiments, the Fc fragment is an IgG1 Fc fragment. In some embodiments, the IgG1 Fc fragment comprises a L234A mutation and/or a L235A mutation. In some embodiments the IgG1 Fc fragment comprises a L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation. In some embodiments, the Fc fragment comprises a N297A mutation. In some embodiments, the Fc fragment comprises a N297G mutation.

In some embodiments, one or more amino acid modifications may be introduced into the Fc region of the antibody moiety, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, the Fc fragment possesses some but not all effector functions, which make it a desirable candidate for applications in which the half-life of the antibody moiety in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (See Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). In some embodiments, the Fc fragment comprises a N297A mutation. In some embodiments, the Fc fragment comprises a N297G mutation.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, the Fc fragment is an IgG1 Fc fragment. In some embodiments, the IgG1 Fc fragment comprises a L234A mutation and/or a L235A mutation. In some embodiments the IgG1 Fc fragment comprises a L235A mutation and/or a G237A mutation. In some embodiments, the Fc fragment is an IgG2 or IgG4 Fc fragment. In some embodiments, the Fc fragment is an IgG4 Fc fragment comprising a S228P, F234A, and/or a L235A mutation.

In some embodiments, the antibody moiety comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some embodiments, the Fc fragment has one or more mutations at Thr250, Met252, Ser254, The256, Thr307. Glu 380, Met428, His433, and/or Asn 434.

In some embodiments, the anti-IGFBP7 construct comprises a variant Fc region comprising one or more amino acid substitutions which alters half-life and/or changes binding to the neonatal Fc receptor (FcRn). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which alters binding of the Fc region to FcRn. Such Fc variants include those with substitutions at one or more of Fc region residues, e.g., substitution of Fc region residue 434 (U.S. Pat. No. 7,371,826).

See also Duncan & Winter, Nature 322:738-40 (1988); U.S. Pat. Nos. 5,648,260; 5,624,821; 8,163,881; and WO 94/29351 concerning other examples of Fc region variants.

h) Cysteine Engineered Antibody Variants

In some embodiments, it may be desirable to create cysteine engineered antibody moieties, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: A118 (EU numbering) of the heavy chain; and S400 (EU numbering) of the heavy chain Fc region. Cysteine engineered antibody moieties may be generated as described, e.g., in U.S. Pat. No. 7,521,541.

i) Antibody Derivatives

In some embodiments, the anti-IGFBP7 antibody moiety or construct described herein may be further modified to comprise additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in diagnosis under defined conditions, etc.

In some embodiments, the anti-IGFBP7 antibody moiety or construct may be further modified to comprise one or more biologically active protein, polypeptides or fragments thereof. “Bioactive” or “biologically active”, as used herein interchangeably, means showing biological activity in the body to carry out a specific function. For example, it may mean the combination with a particular biomolecule such as protein, DNA, etc., and then promotion or inhibition of the activity of such biomolecule. In some embodiments, the bioactive protein or fragments thereof include proteins and polypeptides that are administered to patients as the active drug substance for prevention of or treatment of a disease or condition, as well as proteins and polypeptides that are used for diagnostic purposes, such as enzymes used in diagnostic tests or in vitro assays, as well as proteins and polypeptides that are administered to a patient to prevent a disease such as a vaccine.

### III. Methods of Preparation

In some embodiments, there is provided a method of preparing an anti-IGFBP7 construct or antibody moiety that specifically binds to IGFBP7 and a composition such as polynucleotide, nucleic acid construct, vector, host cell, or culture medium that is produced during the preparation of the anti-IGFBP7 construct or antibody moiety. The anti-IGFBP7 construct or antibody moiety or composition described herein may be prepared by a number of processes as generally described below and more specifically in the Examples.

### Antibody Expression and Production

The anti-IGFBP7 constructs or a portion thereof (such as anti-IGFBP7 antibody moieties, such as anti-IGFBP7 bispecific antibodies) described herein can be prepared using any known methods in the art, including those described below and in the Examples.

Single Domain Antibodies (sdAb)

Methods of preparing sdAbs have been described, see, for example, Els Pardon et al., Nature Protocol, 2014; 9(3): 674. sdAbs (such as V_{H}Hs) may be obtained using methods known in the art such as by immunizing a Camelid species (such as camel or llama) and obtaining hybridomas therefrom, or by cloning a library of single-domain antibodies using molecular biology techniques known in the art and subsequent selection by ELISA with individual clones of unselected libraries or by using phage display.

For recombinant production of the sdAbs, the nucleic acids encoding the single-domain antibodies are isolated and inserted into a replicable vector for further cloning (amplification of the DNA) or for expression. DNA encoding the single-domain antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin, including those described below.

### Monoclonal Antibodies

Monoclonal antibodies or antibody moieties can be obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster or a llama, is immunized as hereinabove described to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986). Also see Example 1 for immunization in Llamas.

The immunizing agent will typically include the antigenic protein or a fusion variant thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, Calif. USA, and SP-2 cells (and derivatives thereof, e.g., X₆3-Ag8-653) available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen. Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein G-Sephatose, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, HEK cells, or myeloma cells (e.g., NS0 cells) that do not otherwise produce immunoglobulin protein, in order to synthesize monoclonal antibodies in such recombinant host cells. Generally, preferred host cells are of either prokaryotic or eukaryotic (generally mammalian) origin, including those described below. Review articles on recombinant expression in bacteria of DNA encoding the antibody include Skerra et al., Curr. Opinion in Immunol., 5:256-262 (1993) and Pluckthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokaryotic Cells

### a) Vector Construction

Polynucleic acid sequences encoding the antibodies of the present application can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

The present invention provides an expression system in which the quantitative ratio of expressed polypeptide components can be modulated in order to maximize the yield of secreted and properly assembled the antibodies of the present application. Such modulation is accomplished at least in part by simultaneously modulating translational strengths for the polypeptide components. One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleic acid sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank.

Preferably, a set of vectors is generated with a range of TIR strengths for each cistron therein. This limited set provides a comparison of expression levels of each chain as well as the yield of the desired protein products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the present application.

### b) Prokaryotic Host Cells

Prokaryotic host cells suitable for expressing the antibodies of the present application include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In some embodiments, E. coli cells are used as hosts for the invention. Examples of E. coli strains include strain W3110 and derivatives thereof, including strain 33D3 having genotype W3110 AfhuA (AtonA) ptr3 lac Iq lacL8 AompT A(nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically, the host cell should secrete minimal amounts of proteolytic enzymes, and additional protease inhibitors may desirably be incorporated in the cell culture.

### c) Protein Production

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

### d) Protein Purification

The construct produced herein or a portion thereof is further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, Protein A immobilized on a solid phase is used for immunoaffinity purification of the antibodies comprising an Fc region of the present application. Protein A is a 411 (D cell wall protein from Staphylococcus aureas which binds with a high affinity to the Fc region of antibodies. Lindmark et al (1983) J. Immunol. Meth. 62:1-13. The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants. The solid phase is then washed to remove contaminants non-specifically bound to the solid phase. Finally the antibodies of interest are recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

### a) Signal Sequence Component

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibodies of the present application.

### b) Origin of Replication

Generally, the origin of replication component is not needed for mammalian expression vectors (the SV40 origin may typically be used only because it contains the early promoter).

### c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

### d) Promoter Component

Expression and cloning vectors usually contain a promoter that is recognized by the host organism and is operably linked to the nucleic acid encoding the desired polypeptide sequences. Virtually all eukaryotic genes have an AT-rich region located approximately 25 to 30 based upstream from the site where transcription is initiated. Another sequence found 70 to 80 bases upstream from the start of the transcription of many genes is a CNCAAT region where N may be any nucleotide. At the 3′ end of most eukaryotic is an AATAAA sequence that may be the signal for addition of the poly A tail to the 3′ end of the coding sequence. All of these sequences may be inserted into eukaryotic expression vectors.

Other promoters suitable for use with prokaryotic hosts include the phoA promoter, -lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the antibodies.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

### e) Enhancer Element Component

Transcription of a DNA encoding the antibodies of the present application by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (100-270 bp), the cytomegalovirus early promoter enhancer, the polyoma enhancer on the late side of the replication origin, and adenovirus enhancers. The enhancer may be spliced into the vector at a position 5′ or 3′ to the polypeptide encoding sequence, but is preferably located at a site 5′ from the promoter.

### f) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region. See WO94/11026 and the expression vector disclosed therein.

### g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natd. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibodies production and cultured in conventional nutrient media modified as appropriate for inducing promoters, selecting transformants, or amplifying the genes encoding the desired sequences.

### h) Culturing the Host Cells

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. The media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In some embodiments, the glutamine synthetase (GS)-CHO expression system is used for culturing the host cells and/or expressing the anti-IGFBP7 construct described herein or a portion thereof. See e.g., Fan et al., J Biotechnol. 2013 December; 168(4):652-8.

### i) Protein Purification

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, multimodal chromatography, ion exchange chromatography, and affinity chromatography, with affinity chromatography being the preferred purification technique. The suitability of protein A as an affinity ligand depends on the species and isotype of any immunoglobulin Fc domain that is present in the antibody. Protein A can be used to purify the antibodies that are based on human immunoglobulins containing 1, 2, or 4 heavy chains. Protein G is recommended for all mouse isotypes and for human 3. The matrix to which the affinity ligand is attached is most often agarose, but other matrices are available. Mechanically stable matrices such as controlled pore glass or poly(styrene-divinyl)benzene allow for faster flow rates and shorter processing times than can be achieved with agarose. Where the antibody comprises a C_{H}3 domain, the Bakerbond ABXTMresin (J. T. Baker, Phillipsburg, N.J.) is useful for purification. Other techniques for protein purification such as ultrafiltration/diafiltration (UF/DF), fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatography on hydrophobic resin, chromatography on mixed-mode resin, chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt). Ion exchange is often used for a polishing step as well.

### Humanized Antibodies

Humanized forms of non-human antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, rabbit, camelid, or llama having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence. In some embodiments, the humanized antibody will comprise at least a portion of an immunoglobulin constant region (Fc), typically that of a human immunoglobulin. See, e.g., Jones et al., Nature, 321: 522-525 (1986); Riechmann et al., Nature, 332: 323-329 (1988); Presta, Curr. Op. Struct. Biol., 2:593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies.

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

In some embodiments, the sdAbs are modified, such as humanized, without diminishing the native affinity of the domain for antigen and while reducing its immunogenicity with respect to a heterologous species. For example, the amino acid residues of the antibody variable domain (VHH) of a llama antibody can be determined, and one or more of the Camelidae amino acids, for example, in the framework regions, are replaced by their human counterpart as found in the human consensus sequence, without that polypeptide losing its typical character, i.e. the humanization does not significantly affect the antigen binding capacity of the resulting polypeptide. Humanization of Camelidae sdAbs requires the introduction and mutagenesis of a limited amount of amino acids in a single polypeptide chain. This is in contrast to humanization of scFv, Fab′, (Fab′)2 and IgG, which requires the introduction of amino acid changes in two chains, the light and the heavy chain and the preservation of the assembly of both chains.

### Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge.

Alternatively, phage display technology can be used to identify human antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991).

Human antibodies may also be generated by in vitro activated B cells (see U.S. Pat. Nos. 5,567,610 and 5,229,275) or by using various techniques known in the art, including phage display libraries (Hoogenboom and Winter, J. Mol. Biol., 227:381 (1991); Marks et al., J. Mol. Biol., 222:581 (1991).

### Nucleic Acid Molecules Encoding Antibody Moieties

In some embodiments, there is provided a polynucleotide encoding any one of the anti-IGFBP7 constructs or antibody moieties described herein. In some embodiments, there is provided a polynucleotide prepared using any one of the methods as described herein. In some embodiments, a nucleic acid molecule comprises a polynucleotide that encodes any of the anti-IGFBP7 single domain antibody (sdAb) moiety described herein. In some embodiments, the polynucleotide comprises a nucleotide sequence that encodes a leader sequence, which, when translated, is located at the N terminus of the sdAb.

In some embodiments, the polynucleotide is a DNA. In some embodiments, the polynucleotide is an RNA. In some embodiments, the RNA is an mRNA.

Nucleic acid molecules may be constructed using recombinant DNA techniques conventional in the art. In some embodiments, a nucleic acid molecule is an expression vector that is suitable for expression in a selected host cell.

### Nucleic Acid Construct

In some embodiments, there is provided a nucleic acid construct comprising any one of the polynucleotides described herein. In some embodiments, there is provided a nucleic acid construct prepared using any method described herein.

In some embodiments, the nucleic acid construct further comprises a promoter operably linked to the polynucleotide. In some embodiments, the polynucleotide corresponds to a gene, wherein the promoter is a wild-type promoter for the gene.

### Vectors

In some embodiments, there is provided a vector comprising any polynucleotides that encode any one of the antibody moieties described herein (e.g., anti-IGFBP7 antibody moieties) or nucleic acid construct described herein. In some embodiments, there is provided a vector prepared using any method described herein. Vectors comprising polynucleotides that encode any of anti-IGFBP7 constructs such as anti-IGFBP7 sdAbs, fusion proteins or other forms of constructs described herein are also provided. Such vectors include, but are not limited to, DNA vectors, phage vectors, viral vectors, retroviral vectors, etc.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004).

### Host Cells

In some embodiments, there is provided a host cell comprising any polypeptide, nucleic acid construct and/or vector described herein. In some embodiments, there is provided a host cell prepared using any method described herein. In some embodiments, the host cell is capable of producing any of antibody moieties described herein under a fermentation condition.

In some embodiments, the antibody moieties described herein (e.g., anti-IGFBP7 antibody moieties) may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, FUT8 CHO cells, and CHO GS cells (Sigma); PER.C6® cells (Crucell); and NSO cells. In some embodiments, the antibody moieties described herein (e.g., anti-IGFBP7 antibody moieties) may be expressed in yeast. See, e.g., U.S. Publication No. US 2006/0270045 A1. In some embodiments, a particular eukaryotic host cell is selected based on its ability to make desired post-translational modifications to the antibody moiety or construct. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Non-limiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

The present application also provides host cells comprising any of the polynucleotides or vectors described herein. In some embodiments, the invention provides a host cell comprising an anti-IGFBP7 antibody. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

In some embodiments, the antibody moiety is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Culture Medium

In some embodiments, there is provided a culture medium comprising any antibody moiety, polynucleotide, nucleic acid construct, vector, and/or host cell described herein. In some embodiments, there is provided a culture medium prepared using any method described herein.

In some embodiments, the medium comprises hypoxanthine, aminopterin, and/or thymidine (e.g., HAT medium). In some embodiments, the medium does not comprise serum. In some embodiments, the medium is chemically defined. In some embodiments, the medium comprises serum. In some embodiments, the medium is a D-MEM or RPMI-1640 medium.

### Purification of Antibody Moieties

The anti-IGFBP7 constructs (e.g., anti-IGFBP7 monoclonal antibodies or multispecific antibodies) may be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, an antibody affinity column, or ultrafiltration/diafiltration (UF/DF) may be used to bind the constant region and/or to purify an anti-IGFBP7 construct comprising an Fc fragment. Hydrophobic interactive chromatography, for example, a butyl or phenyl column, may also suitable for purifying some polypeptides such as antibodies. Ion exchange chromatography (e.g., anion exchange chromatography and/or cation exchange chromatography) may also suitable for purifying some polypeptides such as antibodies. Mixed-mode chromatography (e.g., reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### V. Methods of Treatments

Also provided here are methods of treating a disease or condition in an individual. The methods comprise administering the anti-IGFBP7 construct described herein into individuals (e.g., mammals such as humans).

In some embodiments, there is provided a method of treating a disease or condition (such as a cancer, such as a solid tumor) or inhibiting abnormal vessel growth in a tissue, comprising administering to the individual an effective amount of an anti-IGFBP7 construct (such as any of the anti-IGFBP7 constructs described herein).

In some embodiments, there is provided a method of inhibiting abnormal vessel growth in a tissue in an individual, comprising administering to the individual an effective amount of an anti-IGFBP7 construct (such as any of the anti-IGFBP7 constructs described herein). In some embodiments, the individual has a cancer (such as a solid tumor). In some embodiments, the tissue is a cancer/tumor tissue.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 full-length antibody, wherein the anti-IGFBP7 antibody moiety comprises a single domain antibody (sdAb) moiety, comprising a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the sdAb moiety comprises the amino acid sequence of any of SEQ ID NOs: 32-33 and 46-48, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 full-length antibody, wherein the anti-IGFBP7 antibody moiety comprises a single domain antibody (sdAb) moiety, comprising a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, the sdAb moiety comprises the amino acid sequence of any of SEQ ID NOs: 34 and 49-51, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 full-length antibody, wherein the anti-IGFBP7 antibody moiety comprises a single domain antibody (sdAb) moiety, comprising a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the sdAb moiety comprises the amino acid sequence of any of SEQ ID NOs: 33 and 35-57, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective amount of the anti-IGFBP7 construct comprising an anti-IGFBP7 antibody moiety (such as any of the antibody moiety described herein) fused to the anti-PD-L1 full-length antibody, wherein the anti-IGFBP7 antibody moiety comprises a single domain antibody (sdAb) moiety, comprising a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the sdAb moiety comprises the amino acid sequence of any of SEQ ID NOs: 38 and 39, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 1 or 2, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs. In some embodiments, the sdAb moiety comprises the amino acid sequence any of SEQ ID NOs: 32-33 and 46-48, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs. In some embodiments, the sdAb moiety comprises the amino acid sequence any of SEQ ID NOs: 34 and 49-51, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the sdAb moiety comprises the amino acid sequence any of SEQ ID NOs: 35-37, or a variant thereof having at least about 80% (such as at least about any one of 80%, 85%, 90%, 95%, 96%, 97%, 98%, or 99%) sequence identify.

In some embodiments, there is provided a method of treating a disease or condition (such as a cancer, such as a solid tumor) or inhibiting abnormal vessel growth in a tissue in an individual, comprising administering to the individual an effective mount of the anti-IGFBP7 construct comprising an anti-IGFBP7 antibody moiety (such as any of the antibody moiety described herein) fused to the anti-PD-L1 or anti-PD-1 full-length antibody, wherein the anti-IGFBP7 antibody moiety comprises a single domain antibody (sdAb) moiety, comprising a CDR1 comprising the amino acid sequence of SEQ ID NO: 10 or 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs. In some embodiments, the sdAb moiety comprises the amino acid sequence any of SEQ ID NOs: 38 and 39, or a variant thereof having at least about 80% (such as at least about any one of 80%, 85%, 90%, 95%, 96%, 97%, 98%, or 99%) sequence identify.

In some embodiments, the amino acid substitutions described above are limited to “exemplary substitutions” shown in Table 2 of this application. In some embodiments, the amino acid substitutions are limited to “preferred substitutions” shown in Table 2 of this application.

### Disease or Condition

The methods described herein are applicable to any disease or conditions associated with an abnormal vascular structure. In some embodiments, the disease or condition is an age-related macular degeneration (ARMD). In some embodiments, the disease or condition is a cutaneous psoriasis. In some embodiments, the disease or condition is a benign tumor. In some embodiments, the disease or condition is a cancer.

### Cancer

In some embodiments, the disease or condition described herein is a cancer. Cancers that may be treated using any of the methods described herein include any types of cancers. Types of cancers to be treated with the agent as described in this application include, but are not limited to, carcinoma, blastoma, sarcoma, benign and malignant tumors, and malignancies e.g., sarcomas, carcinomas, and melanomas. Adult tumors/cancers and pediatric tumors/cancers are also included.

In various embodiments, the cancer is early stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

In some embodiments, the cancer is a solid tumor.

In some embodiments, the cancer comprises CD93+ tumor endothelial cells. In some embodiments, at least 10%, 20%, 30%, 40%, 50%, 60%, 70%, 80%, or 90% of the endothelial cells in the tumor are CD93 positive. In some embodiments, the cancer comprises at least 20%, 40%, 60%, 80%, or 100% more CD93+ endothelial cells than that of a normal tissue in the subject. In some embodiments, the cancer comprises at least 20%, 40%, 60%, 80%, or 100% more CD93+ endothelial cells than that of a corresponding organ in a subject or a group of subjects who do not have the cancer.

In some embodiments, the cancer comprises IGFBP7+ blood vessels. In some embodiments, the cancer comprises at least 20%, 40%, 60%, 80%, or 100% more IGFBP7+ blood vessels than that of a normal tissue in the subject. In some embodiments, the cancer comprises at least 20%, 40%, 60%, 80%, or 100% more IGFBP7+ blood vessels than that of a corresponding organ in a subject or a group of subjects who do not have the cancer.

In some embodiments, the cancer (e.g., a solid tumor) is characterized by tumor hypoxia. In some embodiments, the cancer is characterized by a pimonidazole positive percentage (i.e., pimonidazole positive area divided by total tumor area) of at least about 1%, 2%, 3%, 4%, or 5%.

Examples of cancers that may be treated by the methods of this application include, but are not limited to, anal cancer, astrocytoma (e.g., cerebellar and cerebral), basal cell carcinoma, bladder cancer, bone cancer, (osteosarcoma and malignant fibrous histiocytoma), brain tumor (e.g., glioma, brain stem glioma, cerebellar or cerebral astrocytoma (e.g., astrocytoma, malignant glioma, medulloblastoma, and glioblastoma), breast cancer, cervical cancer, colon cancer, colorectal cancer, endometrial cancer (e.g., uterine cancer), esophageal cancer, eye cancer (e.g., intraocular melanoma and retinoblastoma), gastric (stomach) cancer, gastrointestinal stromal tumor (GIST), head and neck cancer, hepatocellular (liver) cancer (e.g., hepatic carcinoma and heptoma), liver cancer, lung cancer (e.g., small cell lung cancer, non-small cell lung cancer, adenocarcinoma of the lung, and squamous carcinoma of the lung), medulloblastoma, melanoma, mesothelioma, myelodysplastic syndromes, nasopharyngeal cancer, neuroblastoma, ovarian cancer, pancreatic cancer, parathyroid cancer, cancer of the peritoneal, pituitary tumor, rectal cancer, renal cancer, renal pelvis and ureter cancer (transitional cell cancer), rhabdomyosarcoma, skin cancer (e.g., non-melanoma (e.g., squamous cell carcinoma), melanoma, and Merkel cell carcinoma), small intestine cancer, squamous cell cancer, testicular cancer, thyroid cancer, and tuberous sclerosis. Additional examples of cancers can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

### Subject

In some embodiments, the subject is a mammal (such as a human).

In some embodiments, the subject has a tissue comprising abnormal vascular comprising CD93+ endothelial cells. In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93+ endothelial cells than that of a normal tissue in the subject. In some embodiments, the tissue with abnormal vascular comprises at least 20%, 40%, 60%, 80%, or 100% more CD93+ endothelial cells than that of a corresponding organ in a subject or a group of subjects who do not have the abnormal vascular.

In some embodiments, the subject has a tissue comprising abnormal vascular comprising IGFBP7+ blood vessels. In some embodiments, the tissue comprises at least 20%, 40%, 60%, 80%, or 100% more IGFBP7+ blood vessels than that of a normal tissue in the subject. In some embodiments, the tissue comprises at least 20%, 40%, 60%, 80%, or 100% more IGFBP7+ blood vessels than that of a corresponding organ in a subject or a group of subjects who do not have the abnormal vascular.

In some embodiments, the subject is selected for treatment based upon an abnormal vascular structure. In some embodiments, the abnormal vascular structure is characterized by CD93+ endothelial cells (for example, by measuring CD93+ CD31+ cells). In some embodiments, at least 10%, 20%, 30%, 40%, 50%, 60%, 70%, 80%, or 90% of the endothelial cells in the tissue with abnormal vascular are CD93 positive. In some embodiments, the tissue with abnormal vascular comprises at least 20%, 40%, 60%, 80%, or 100% more CD93+ endothelial cells than that of a normal tissue in the subject. In some embodiments, the tissue with abnormal vascular comprises at least 20%, 40%, 60%, 80%, or 100% more CD93+ endothelial cells than that of a corresponding organ in a subject or a group of subjects who do not have the abnormal vascular.

In some embodiments, the abnormal vascular structure is characterized by an abnormal level of IGFBP7+ blood vessels. In some embodiments, the tissue comprises at least 20%, 40%, 60%, 80%, or 100% more IGFBP7+ blood vessels than that of a normal tissue in the subject. In some embodiments, the tissue comprises at least 20%, 40%, 60%, 80%, or 100% more IGFBP7+ blood vessels than that of a corresponding organ in a subject or a group of subjects who do not have the abnormal vascular.

### Dosing and Method of Administering the Anti-IGFBP7 Construct

The dosing regimen of the anti-IGFBP7 construct (such as the specific dosages and frequencies) used for treating a disease or disorder as described herein administered into the individual may vary with the particular anti-IGFBP7 construct (such as anti-IGFBP7 monoclonal or multispecific antibodies, such as anti-IGFBP7 fusion proteins), the mode of administration, and the type of disease or condition being treated. In some embodiments, the type of disease or condition is a cancer. In some embodiments, the effective amount of the anti-IGFBP7 construct (such as anti-IGFBP7 monoclonal or multispecific antibodies) is an amount that is effective to result in an objective response (such as a partial response or a complete response). In some embodiments, the effective amount of the anti-IGFBP7 construct (such as anti-IGFBP7 monoclonal or multispecific antibodies) is an amount that is sufficient to result in a complete response in the individual. In some embodiments, the effective amount of the anti-IGFBP7 construct (such as anti-IGFBP7 monoclonal or multispecific antibodies) is an amount that is sufficient to result in a partial response in the individual. In some embodiments, the effective amount of anti-IGFBP7 construct (such as anti-IGFBP7 monoclonal or multispecific antibodies) is an amount that is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-IGFBP7 construct (such as anti-IGFBP7 monoclonal or multispecific antibodies). Responses of an individual to the treatment of the methods described herein can be determined, for example, based on RECIST levels.

In some embodiments, the effective amount of the anti-IGFBP7 construct (such as anti-IGFBP7 monoclonal or multispecific antibodies) is an amount that is sufficient to prolong progress-free survival of the individual. In some embodiments, the effective amount of the anti-IGFBP7 construct (such as anti-IGFBP7 monoclonal or multispecific antibodies) is an amount that is sufficient to prolong overall survival of the individual. In some embodiments, the effective amount of the anti-IGFBP7 construct (such as anti-IGFBP7 monoclonal or multispecific antibodies) is an amount that is sufficient to produce clinical benefit of more than about any of 50%, 60%, 70%, 80%, or 90% among a population of individuals treated with the anti-IGFBP7 construct (such as anti-IGFBP7 monoclonal or multispecific antibodies).

In some embodiments, the effective amount of the anti-IGFBP7 construct (such as anti-IGFBP7 monoclonal or multispecific antibodies) alone or in combination with a second, third, and/or fourth agent, is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of cancer cells, or tumor growth rate in the same subject prior to treatment or compared to the corresponding activity in other subjects not receiving the treatment (e.g., receiving a placebo treatment). Standard methods can be used to measure the magnitude of this effect, such as in vitro assays with purified enzyme, cell-based assays, animal models, or human testing.

In some embodiments, the effective amount of the anti-IGFBP7 construct (such as anti-IGFBP7 monoclonal or multispecific antibodies) is an amount that is below the level that induces a toxicological effect (i.e., an effect above a clinically acceptable level of toxicity) or is at a level where a potential side effect can be controlled or tolerated when the composition is administered to the individual.

In some embodiments, the effective amount of the anti-IGFBP7 construct (such as anti-IGFBP7 monoclonal or multispecific antibodies) is an amount that is close to a maximum tolerated dose (MTD) of the composition following the same dosing regimen. In some embodiments, the effective amount of the anti-IGFBP7 construct (such as anti-IGFBP7 monoclonal or multispecific antibodies) is more than about any of 80%, 90%, 95%, or 98% of the MTD.

In some embodiments, the effective amount of the anti-IGFBP7 construct (such as anti-IGFBP7 monoclonal or multispecific antibodies) is an amount that slows or inhibits the progression of the disease or condition (for example, by at least about 5%, 10%, 15%, 20%, 30%, 40%, 50%) as compared to that of the individual not receiving the treatment. In some embodiments, the disease or condition is an autoimmune disease. In some embodiments, the disease or condition is an infection.

In some embodiments, the effective amount of the anti-IGFBP7 construct (such as anti-IGFBP7 monoclonal or multispecific antibodies) is an amount that reduces the side effects (auto-immune response) of a condition (e.g., transplantation) (for example, by at least about 5%, 10%, 15%, 20%, 30%, 40%, or 50%) as compared to that of the individual not receiving the treatment.

In some embodiments of any of the above aspects, the effective amount of an anti-IGFBP7 construct (such as anti-IGFBP7 monoclonal or multispecific antibodies) is in the range of about 0.001 μg/kg to about 100 mg/kg of total body weight, for example, about 0.005 μg/kg to about 50 mg/kg, about 0.01 μg/kg to about 10 mg/kg, or about 0.01 μg/kg to about 1 mg/kg.

The anti-IGFBP7 construct can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, the anti-IGFBP7 construct is included in a pharmaceutical composition while administered into the individual. In some embodiments, sustained continuous release formulation of the composition may be used. In some embodiments, the composition is administered intravenously. In some embodiments, the composition is administered intraperitoneally. In some embodiments, the composition is administered intravenously. In some embodiments, the composition is administered intraperitoneally. In some embodiments, the composition is administered intramuscularly. In some embodiments, the composition is administered subcutaneously. In some embodiments, the composition is administered intravenously. In some embodiments, the composition is administered orally. In some embodiments, the composition is administered intravitreally (e.g., for treating age-related macular degeneration (ARMD)).

In some embodiments, the anti-IGFBP7 construct (e.g., A1 mIgG2a-D4) is administered at a frequency of about once a week to every two days. In some embodiments, the anti-IGFBP7 construct (e.g., A1 mIgG2a-D4) is administered at a frequency of about once every three to four days.

In some embodiments, the anti-IGFBP7 construct is a bispecific IgG nanobody (e.g., A1 mIgG2a-D4). In some embodiments, the bispecific IgG nanobody (e.g., A1 mIgG2a-D4) is administered at a dose for a human that is equivalent to about 0.3 mg for a mouse. See Nair et al., J Basic Clin Pharm. March 2016-May 2016; 7(2): 27-31. In some embodiments, the bispecific IgG nanobody (e.g., A1 mIgG2a-D4) is administered at a dose about 75 mg for human. In some embodiments, the anti-IGFBP7 construct (e.g., A1 mIgG2a-D4) is administered at a dose about 45 mg/m² or 1.2 mg/kg for human.

### Combination Therapy

This application also provides methods of administering an anti-IGFBP7 construct into an individual for treating a disease or condition (such as cancer), wherein the method further comprises administering a second agent or therapy. In some embodiments, the second agent or therapy is a standard or commonly used agent or therapy for treating the disease or condition. In some embodiments, the second agent or therapy comprises a chemotherapeutic agent. In some embodiments, the second agent or therapy comprises a surgery. In some embodiments, the second agent or therapy comprises a radiation therapy. In some embodiments, the second agent or therapy comprises an immunotherapy. In some embodiments, the second agent or therapy comprises a cell therapy (such as a cell therapy comprising an immune cell (e.g., CAR T cell)). In some embodiments, the second agent or therapy comprises an angiogenesis inhibitor.

In some embodiments, the second agent is a chemotherapeutic agent. In some embodiments, the second agent is antimetabolite agent. In some embodiments, the antimetabolite agent is 5-FU.

In some embodiments, the second agent is an immune checkpoint modulator. In some embodiments, the immune checkpoint modulator is an inhibitor of an immune checkpoint protein selected from the group consisting of PD-L1, PD-L2, CTLA4, PD-L2, PD-1, CD47, TIGIT, GITR, TIM3, LAG3, CD27, 4-1BB, CD96, PVRIG, and B7H4. In some embodiments, the immune checkpoint protein is PD-1. In some embodiments, the second agent is an anti-PD-1 antibody or fragment thereof.

In some embodiments, the second therapy is an immunotherapy. In some embodiments, the immunotherapy comprises administering an immune cell expressing a chimeric antigen receptor. In some embodiments, the immune cell is a T cell (such as a CD4+ T cell or a CD8+ T cell). In some embodiments, the chimeric antigen receptor binds to a tumor antigen.

In some embodiments, the anti-IGFBP7 construct is administered simultaneously with the second agent or therapy. In some embodiments, the anti-IGFBP7 construct is administered concurrently with the second agent or therapy. In some embodiments, the anti-IGFBP7 construct is administered sequentially with the second agent or therapy. In some embodiments, the anti-IGFBP7 construct is administered prior to the second agent or therapy. In some embodiments, the anti-IGFBP7 construct is administered after the second agent or therapy. In some embodiments, the anti-IGFBP7 construct is administered in the same unit dosage form as the second agent or therapy. In some embodiment, the anti-IGFBP7 construct is administered in a different unit dosage form from the second agent or therapy. In some embodiments, the anti-IGFBP7 construct is administered in the same unit dosage form as the second agent or therapy. In some embodiment, the anti-IGFBP7 construct is administered in a different unit dosage form from the second agent or therapy.

### VI. Compositions, Kits and Articles of Manufacture

Also provided herein are compositions (such as formulations) comprising any one of the anti-IGFBP7 construct or anti-IGFBP7 antibody moiety described herein, nucleic acid encoding the antibody moieties, vector comprising the nucleic acid encoding the antibody moieties, or host cells comprising the nucleic acid or vector.

Suitable formulations of the anti-IGFBP7 construct described herein can be obtained by mixing the anti-IGFBP7 construct or anti-IGFBP7 antibody moiety having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be imaged, diagnosed, or treated herein.

The formulations to be used for in vivo administration must be sterile. This is readily accomplished by, e.g., filtration through sterile filtration membranes.

Also provided are kits comprising any one of the anti-IGFBP7 construct or anti-IGFBP7 antibody moiety described herein. The kits may be useful for any of the methods of modulating cell composition or treatment described herein.

In some embodiments, there is provided a kit comprising an anti-IGFBP7 construct specifically binding to IGFBP7.

In some embodiments, the kit further comprises a device capable of delivering the anti-IGFBP7 construct into an individual. One type of device, for applications such as parenteral delivery, is a syringe that is used to inject the composition into the body of a subject.

Inhalation devices may also be used for certain applications.

In some embodiments, the kit further comprises a therapeutic agent for treating a disease or condition, e.g., cancer, infectious disease, autoimmune disease, or transplantation.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information.

The present application thus also provides articles of manufacture. The article of manufacture can comprise a container and a label or package insert on or associated with the container. Suitable containers include vials (such as sealed vials), bottles, jars, flexible packaging, and the like. Generally, the container holds a composition, and may have a sterile access port (for example the container may be an intravenous solution bag or a vial having a stopper pierceable by a hypodermic injection needle). The label or package insert indicates that the composition is used for imaging, diagnosing, or treating a particular condition in an individual. The label or package insert will further comprise instructions for administering the composition to the individual and for imaging the individual. The label may indicate directions for reconstitution and/or use. The container holding the composition may be a multi-use vial, which allows for repeat administrations (e.g., from 2-6 administrations) of the reconstituted formulation. Package insert refers to instructions customarily included in commercial packages of diagnostic products that contain information about the indications, usage, dosage, administration, contraindications and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the compositions and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Exemplary Embodiments

Embodiment 1. An anti-IGFBP7 construct comprising a polypeptide comprising a single domain antibody (sdAb) moiety specifically recognizing IGFBP7, wherein the sdAb moiety comprises:
1) a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
2) a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
3) a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs;
4) a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
5) a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
6) a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
7) a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
8) a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs;
9) a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs;
10) a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs;
11) a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs;
12) a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs; or
13) a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs.

Embodiment 2. An anti-IGFBP7 construct comprising a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in any of SEQ ID NOs: 32-51.

Embodiment 3. The anti-IGFBP7 construct of Embodiment 1 or 2, wherein the sdAb moiety comprises the amino acid sequence of any one of SEQ ID NOs: 32-51, or a variant thereof having at least about 80% sequence identify to any one of SEQ ID NOs: 32-51.

Embodiment 4. The anti-IGFBP7 construct of any one of Embodiments 1-3, wherein the sdAb moiety is camelid, chimeric, human, partially humanized, or fully humanized.

Embodiment 5. The anti-IGFBP7 construct of any one of Embodiments 1-4, wherein the sdAb moiety is a V_{H}H antibody.

Embodiment 6. The anti-IGFBP7 construct of any one of Embodiments 1-5, wherein anti-IGFBP7 construct blocks the binding of CD93 to IGFBP7.

Embodiment 7. The anti-IGFBP7 construct of any one of Embodiments 1-6, wherein the IGFBP7 is human IGFBP7.

Embodiment 8. The anti-IGFBP7 construct of Embodiment 6 or Embodiment 7, wherein the CD93 is human CD93.

Embodiment 9. The anti-IGFBP7 construct of any one of Embodiments 1-8, wherein the anti-IGFBP7 construct further comprises a second moiety.

Embodiment 10. The anti-IGFBP7 construct of Embodiment 9, wherein the second moiety comprises an antibody moiety that specifically recognizes an antigen.

Embodiment 11. The anti-IGFBP7 construct of Embodiment 10, wherein the antigen is PD-L1 or PD-1.

Embodiment 12. The anti-IGFBP7 construct of Embodiment 10 or 11, wherein the second antibody moiety is a full-length antibody, a Fab, a Fab′, a (Fab′)₂, an Fv, a single chain Fv (scFv), an scFv-scFv, a minibody, a diabody, or an sdAb.

Embodiment 13. The anti-IGFBP7 construct of Embodiment 9, where the second moiety comprises a half-life extending moiety.

Embodiment 14. The anti-IGFBP7 construct of Embodiment 9, wherein the construct is an antibody-drug conjugate.

Embodiment 15. An anti-IGFBP7 construct that specifically binds to IGFBP7 competitively with the anti-IGFBP7 construct of any one of Embodiments 1-14.

Embodiment 16. A pharmaceutical composition comprising the anti-IGFBP7 construct of any one of Embodiments 1-15 and a pharmaceutical acceptable carrier.

Embodiment 17. A polynucleotide encoding the polypeptide of the anti-IGFBP7 construct of any one of Embodiments 1-15 or a portion thereof.

Embodiment 18. A nucleic acid construct, comprising the polynucleotide of Embodiment 17, optionally further comprising a promoter in operative connection with the polynucleotide.

Embodiment 19. A vector comprising the nucleic acid construct of Embodiment 18.

Embodiment 20. An isolated host cell comprising the polynucleotide according to Embodiment 18, the nucleic acid construct according to Embodiment 18, or the vector according to Embodiment 19.

Embodiment 21. A culture medium comprising the polypeptide of anti-IGFBP7 construct of Embodiment any one of Embodiments 1-15, the polynucleotide according to Embodiment 17, the nucleic acid construct according to Embodiment 18, the vector according to Embodiment 19, or the host cell according to Embodiment 20.

Embodiment 22. A method of producing an anti-IGFBP7 construct, comprising:
a) culturing the isolated host cell of Embodiment 20 under conditions effective to express the polypeptide; and
b) obtaining the polypeptide from the host cell.

Embodiment 23. A method of treating a disease or condition (such as a cancer, such as a solid tumor) or inhibiting abnormal vessel growth in a tissue in an individual, comprising administering to the individual an effective mount of the anti-IGFBP7 construct of any one of Embodiments 1-15, or the pharmaceutical composition of Embodiment 16.

Embodiment 24. The method of Embodiment 23, wherein the disease of condition is associated with an abnormal vascular structure.

Embodiment 25. The method of Embodiment 23 or Embodiment 24, wherein the disease or condition is a cancer.

Embodiment 26. The method of Embodiment 25, wherein the cancer is a solid tumor.

Embodiment 27. The method of Embodiment 25 or Embodiment 26, wherein the cancer comprises CD93+ endothelial cells.

Embodiment 28. The method of any one of Embodiments 25-27, wherein the cancer comprises IGFBP7+ blood vessels.

Embodiment 29. The method of any one of Embodiments 25-28, wherein the cancer is characterized by tumor hypoxia.

Embodiment 30. The method of any one of Embodiments 25-29, wherein the cancer is a locally advanced or metastatic cancer.

Embodiment 31. The method of any one of Embodiments 25-30, wherein the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

Embodiment 32. The method of any one of Embodiments 23-31, wherein the anti-IGFBP7 construct is administered parenterally into the individual.

Embodiment 33. The method of any one of Embodiments 23-32, wherein the method further comprises administering a second therapy.

Embodiment 34. The method of Embodiment 33, wherein the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy.

Embodiment 35. The method of Embodiment 34, wherein the second therapy is an immunotherapy.

Embodiment 36. The method of Embodiment 35, wherein the immunotherapy comprises administering an immunomodulatory agent.

Embodiment 37. The method of Embodiment 36, wherein the immunomodulatory agent is an immune checkpoint inhibitor.

Embodiment 38. The method of Embodiment 37, wherein the immune checkpoint inhibitor comprises an anti-PD-L1 antibody or an anti-PD-1 antibody.

Embodiment 39. The method of any one of Embodiments 23-38, wherein the individual is a human.

### EXAMPLES

The examples below are intended to be purely exemplary of the application and should therefore not be considered to limit the application in any way. The following examples and detailed description are offered by way of illustration and not by way of limitation.

### Materials

Biotinylated human IGFBP7 was generated using standard protocols (EZ-Link™ Sulfo-NHS-SS-Biotin; Thermo Fisher Scientific, Catalog #21331).

Goat anti-Llama IgG (H+L) Secondary Antibody, HRP was obtained from Thermo Fisher Scientific, Catalog #A16060.

Anti-M13 Secondary Antibody, HRP was obtained from GE, Catalog #27-9421-01.

His tagged murine N-terminal IGFBP7₂₈₋₁₀₆ and human N-terminal IGFBP7₂₈₋₁₀₆-llama Fc were generated using standard protocol.

### Example 1. Immunizations

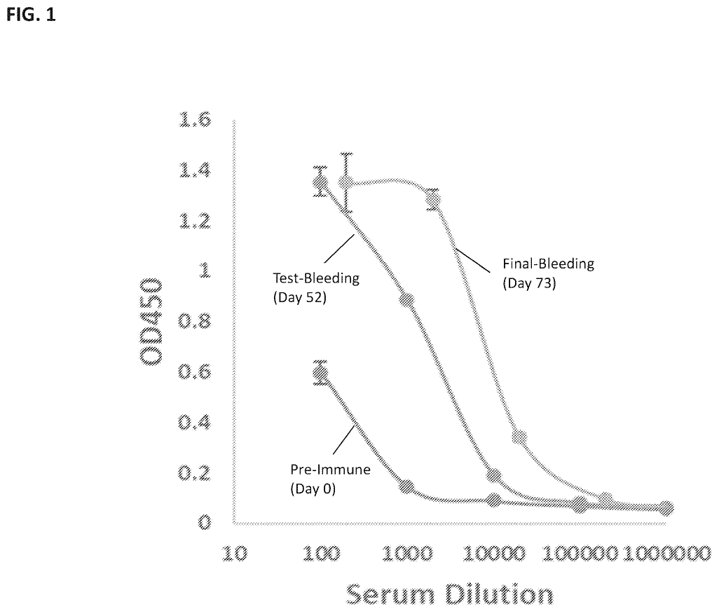

One llama was immunized with human IGFBP7 (hIGFBP7) according to the scheme outlined in Table 3. After completion of the protocol, immune responses were analyzed by ELISA. Specifically, serum samples were collected at days 0 and day 52, and incubated with human IGFBP7 (0.5 μg/mL in PBS) or negative Fc protein adsorbed to a 96-well ELISA plate. Bound llama IgG was detected by goat anti-Llama IgG (Bethyl A160-100; Montgomery, TX). Results at day 52 are shown in FIG. 1.

TABLE 3
Immunization schedules

| | | |
|---|---|---|
| Injections: | Inject 1000 μg CFA | Day 0 |
| | Inject 500 μg IFA | Day 21 |
| | Inject 500 μg IFA | Day 42 |
| | Inject 500 μg IFA | Day 63 |
| Bleeds: | Pre Bleed | Day 0 |
| | Test Bleed | Day 52 |
| | Production Bleed | Day 73 |

As shown in FIG. 1, hIGFBP7 effectively induces anti-human IGFBP7 antibody production in the test llama, in llama serum diluted up to 10⁷-fold.

### Example 2. Library Construction

RNA extracted from peripheral blood mononuclear cell (PBMC) obtained from llama was used as starting material for RT-PCR to amplify nanobodies encoding gene fragments. These fragments were cloned into a phagemid vector. Phage was prepared according to standard methods and stored after filter sterilization at 4° C. for further use.

### Example 3. Selections

Selections were carried out with the constructed libraries using standard phage display methods. Two rounds of selection were performed with biotinylated human IGFBP7 (in solution, followed by capturing on streptavidin beads, followed by washing and trypsin elution). Each selection output was analyzed for enrichment factor (e.g., number of phage present in eluate relative to control) and plated for further analyses. A third round of selection was performed using the N-terminal tagged human IGFBP7-His.

Colonies were picked, grown in two 96 deep well plates (2 mL volume). One plate was grown up for sequencing and another plate was added helper phage for phage expression with ELISA test.

### Example 4. Primary Screening of Binding to Human IGFBP7 by ELISA

A solution of 50 μL from the phage expressed plate was incubated with hIGFBP7 (0.5 μg/mL in PBS) or negative Fc protein adsorbed to a 96-well ELISA plate. Phage-displayed recombinant antibodies were detected by anti-M13 Secondary Antibody, HRP (GE, Catalog #27-9421-01).

### Example 5. Sequence Analysis

96-well plates were subjected to sequence analysis, resulting in the identification of a set of different, unique nanobody sequences.

Based on the amino acid sequence homology (in particular, homology for CDR3) all clones were classified into different families or single sequence representatives. See, Table 4. Members of the same family are believed to have similar behavior in the different assays described herein, though small differences in functional properties are possible. One or several representatives per family were chosen for further characterization.

TABLE 4
Consensus sequence analysis

| | | | | |
|---|---|---|---|---|
| No. | Clone | CDR1 (Kabat) | CDR2 (Kabat) | CDR3 (Kabat) |

Group A-1

| | | | | |
|---|---|---|---|---|
| 1 | A01 | IDKWG | WRYSPVSINYGDSVKG | VISGAWY |
| | | (SEQ ID NO: 2) | (SEQ ID NO: 3) | (SEQ ID NO: 4) |
| 15 | hA1-1 | IDKWG | WRYSPVSINYGDSVKG | VISGAWY |
| | | (SEQ ID NO: 2) | (SEQ ID NO: 3) | (SEQ ID NO: 4) |
| 16 | hA1-2 | IDKWG | WRYSPVSINYGDSVKG | VISGAWY |
| | | (SEQ ID NO: 2) | (SEQ ID NO: 3) | (SEQ ID NO: 4) |
| 17 | hA1-3 | IDKWG | WRYSPVSINYGDSVKG | VISGAWY |
| | | (SEQ ID NO: 2) | (SEQ ID NO: 3) | (SEQ ID NO: 4) |

| | | | |
|---|---|---|---|
| Group A-1 | IDKWG | WRYSPVSINYGDSVKG | VISGAWY |
| consensus | (SEQ ID NO: 2) | (SEQ ID NO: 3) | (SEQ ID NO: 4) |
| sequences | | | |

Group A-2

| | | | | |
|---|---|---|---|---|
| 2 | G02 | NANKGAMT | WRYSPVSINYGDSVKG | VISGAWY |
| | | (SEQ ID NO: 1) | (SEQ ID NO: 3) | (SEQ ID NO: 4) |

Group B-1

| | | | | |
|---|---|---|---|---|
| 4 | F12 | INTYL | AITSGGSINYADSVKG | KAHPNPWGFDNDY |
| | | (SEQ ID NO: 9) | (SEQ ID NO: 12) | (SEQ ID NO: 13) |
| 5 | A03 | INTYL | AITSGGSINYADSVKG | KAHPNPWGFDNDY |
| | | (SEQ ID NO: 9) | (SEQ ID NO: 12) | (SEQ ID NO: 13) |
| 6 | A04 | INTYL | AITSGGSINYADSVKG | KAHPNPWGFDNDY |
| | | (SEQ ID NO: 9) | (SEQ ID NO: 12) | (SEQ ID NO: 13) |

| | | | |
|---|---|---|---|
| Group B-1 | INTYL | AITSGGSINYADSVKG | KAHPNPWGFDNDY |
| consensus | (SEQ ID NO: 9) | (SEQ ID NO: 12) | (SEQ ID NO: 13) |
| sequences | | | |

Group B-2

| | | | | |
|---|---|---|---|---|
| 7 | A07 | AINGAM | AITSGGSINYADSVKG | KAHPNPWGFDNDY |
| | | (SEQ ID NO: 10) | (SEQ ID NO: 12) | (SEQ ID NO: 13) |
| 8 | A11 | ANNGAM | AITSGGSINYADSVKG | KAHPNPWGFDNDY |
| | | (SEQ ID NO: 11) | (SEQ ID NO: 12) | (SEQ ID NO: 13) |

| | | | |
|---|---|---|---|
| Group B-2 | AXINGAM | AITSGGSINYADSVKG | KAHPNPWGFDNDY |
| consensus | (SEQ ID NO: 53) | (SEQ ID NO: 12) | (SEQ ID NO: 13) |
| sequences | X1 = N or I | | |

Group C

| | | | | |
|---|---|---|---|---|
| 3 | D04 | FSSYAMG | AITWSGDYTYYTDSVKG | KFSQVSAFFATVSEYEY |
| | | (SEQ ID NO: 5) | (SEQ ID NO: 6) | (SEQ ID NO: 7) |
| 18 | hD4-1 | FSSYAMG | AITWSGDYTYYTDSVKG | KFSQVSAFFATVSEYEY |
| | | (SEQ ID NO: 5) | (SEQ ID NO: 6) | (SEQ ID NO: 7) |
| 19 | hD4-2 | FSSYAMG | AITWSGDYTYYTDSVKG | KFSQVSAFFATVSEYEY |
| | | (SEQ ID NO: 5) | (SEQ ID NO: 6) | (SEQ ID NO: 7) |
| 20 | hD4-3 | FSSYAMG | AITWSGDYTYYTDSVKG | KFSQVSAFFATVSEYEY |
| | | (SEQ ID NO: 5) | (SEQ ID NO: 6) | (SEQ ID NO: 7) |

| | | | |
|---|---|---|---|
| Group C | FSSYAMG | AITWSGDYTYYTDSVKG | KFSQVSAFFATVSEYEY |
| consensus | (SEQ ID NO: 5) | (SEQ ID NO: 6) | (SEQ ID NO: 7) |
| sequences | | | |

Ungrouped

| | | | | |
|---|---|---|---|---|
| 9 | B01 | INAMA | SLKYDGSTNYADFAKG | DMTDWGLVAGQFDV |
| | | (SEQ ID NO: 14) | (SEQ ID NO: 15) | (SEQ ID NO: 16) |
| 10 | F09 | INAMG | ALTSGGSTMYGDSVKG | RLIRTINGVDYDY |
| | | (SEQ ID NO: 17) | (SEQ ID NO: 18) | (SEQ ID NO: 19) |
| 11 | F02 | LYAIA | CISATHGEDIVAYRESVKG | DGATCGDLYGRSWDGGP |
| | | (SEQ ID NO: 20) | (SEQ ID NO: 21) | VNRPPQFGS |
| | | | | (SEQ ID NO: 22) |
| 12 | C03 | AYAMG | SISWTGSTSYYADSVKG | TTYFRGTYNVESAFGS |
| | | (SEQ ID NO: 23) | (SEQ ID NO: 24) | (SEQ ID NO: 25) |
| 13 | A08 | SYVMG | AIPWDDDWTRYEDFVSG | STRPGAPYVY |
| | | (SEQ ID NO: 26) | (SEQ ID NO: 27) | (SEQ ID NO: 28) |
| 14 | E12 | TYYMT | AINTGGGTTYYADSVKG | GDYYESTYYPRY |
| | | (SEQ ID NO: 29) | (SEQ ID NO: 30) | (SEQ ID NO: 31) |

### Example 6. Nanobody Expression and Purification in E. coli

Selected nanobodies were expressed in E. coli as His6-tagged proteins in a culture volume of 30 mL, and expression was induced with auto-induction mTB media for 48 hours at 37° C. After spinning the cell cultures, pellets were incubated with lysozyme and 10% CHAPS for 30 minutes at room temperature. These extracts were centrifuged at 12000×g and the solutions were loaded to a Ni-t column with standard purification methods. Nanobodies were eluted from the column with 150 mM imidazole and subsequently buffer exchanged to PBS.

### Example 7. Binding of Anti-IGFBP7 Nanobodies to hIGFBP7 and mIGFBP7 by FACS

Human IGFBP7 and mouse IGFBP7 displaying HEK293T cells were detached by incubation with TrypLE reagents (Thermos Fisher), which preserves the integrity of IGFBP7 on the cell surface. Then the cells were incubated with anti-IGFBP7 nanobodies at 10 μg/ml for 30 minutes in 4° C. After washing by FACS buffer, the cells were incubated with APC conjugated anti-HIS antibodies (Biolegend) for 30 minutes in 4° C. After washing by FACS buffer twice, the samples were acquired in NovoCyte Flow Cytometer and analyzed by NovoExpress software.

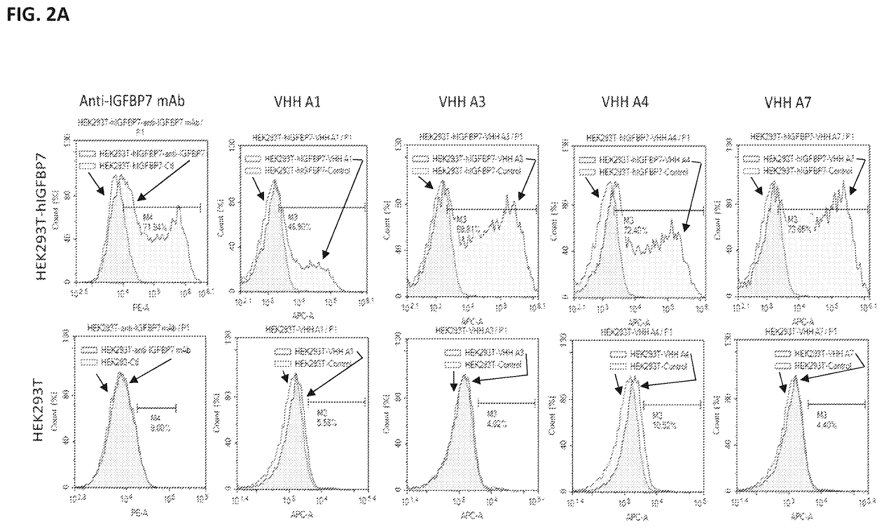

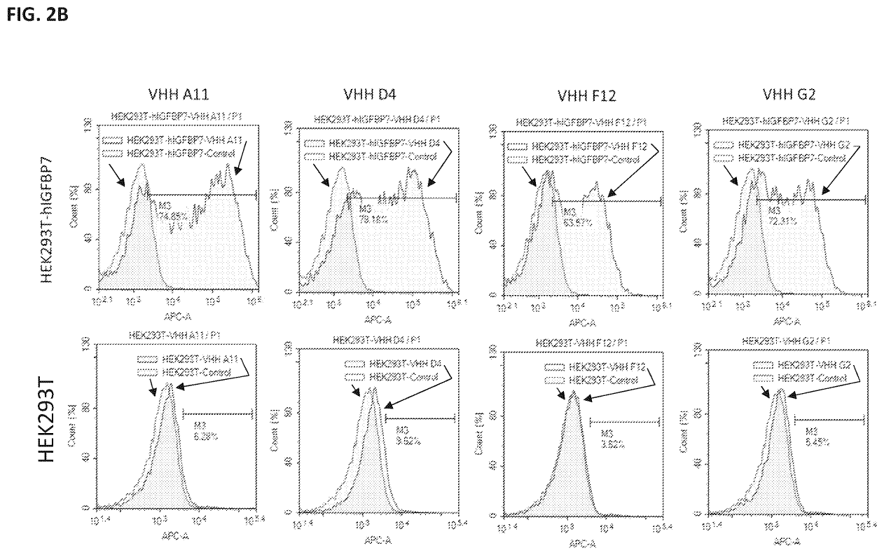

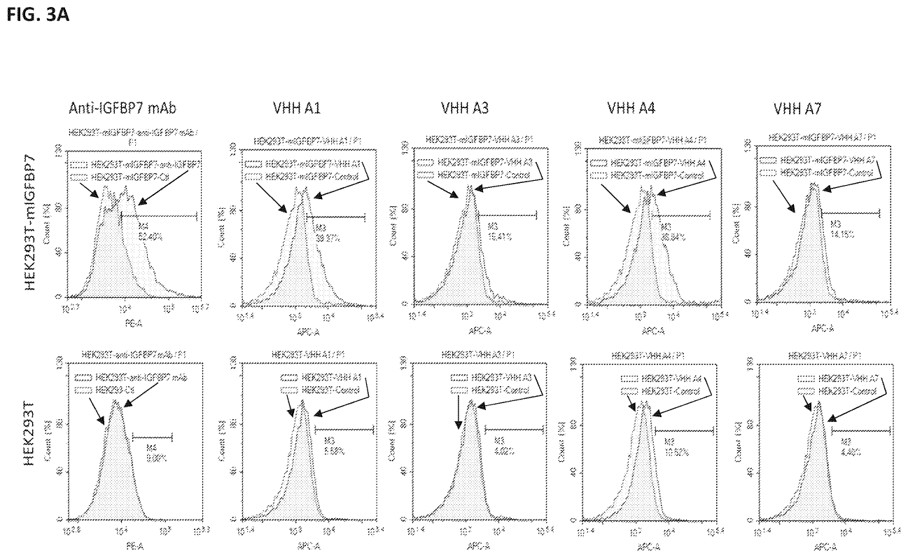

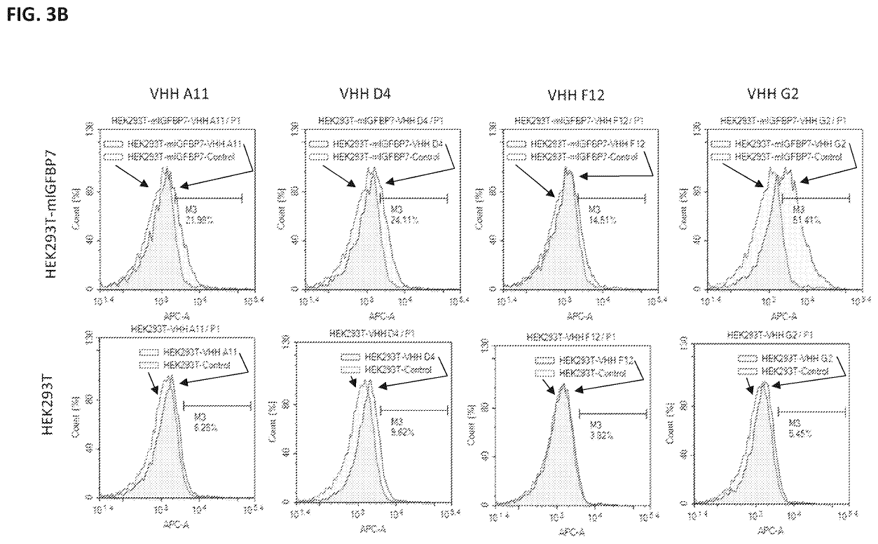

The results are shown in FIGS. 2A-2B and 3A-3B.

As shown in FIGS. 2A-2B, clones A1, A3, A4, A7, A11, D4, F12, and G2 all bind hIGFBP7. As shown in FIGS. 3A-3B, clones A1, A4, A11, D4, and G2 all bind mIGFBP7.

### Example 8. Recombinant Expression of Nanobody Fc-Fusions

Anti-IGFBP7 nanobodies that bound to both hIGFBP7 and mIGFBP7 by ELISA (e.g., A1, A4, A11, and D4 V_{H}H) were fused with mIgG2a or mIgG1 Fc fragment (i.e., nanobody Fc-fusion), and then were cloned and expressed in Expi293 cells. Briefly, the mouse IgG Fc-fucion nanobodies were gene synthesized using human preferred codons (IDT). Then the gene fragments were subcloned into the pcDNA3.4 vector which contains the murine antibody signal sequences and mIgG2a or mIgG1 Fc fragment. The nanobody Fc-fusion proteins were produced by transient transfection into Expi293 cells using ExpiFectamine 293 transfection kit (Thermo Fisher Scientific). Five days after transfection, the supernatants from transfected cells were collected and purified using Protein G Sepharose (GE). The bound antibodies were eluted using 0.1 M Glycine buffer (pH 2.7) and dialyzed with 1×PBS (pH 7.4) overnight. The purified antibodies were analyzed on reduced and non-reduced SDS-PAGE to confirm the purity and size. Protein concentration was determined by A₂₈₀ on a spectrophotometer.

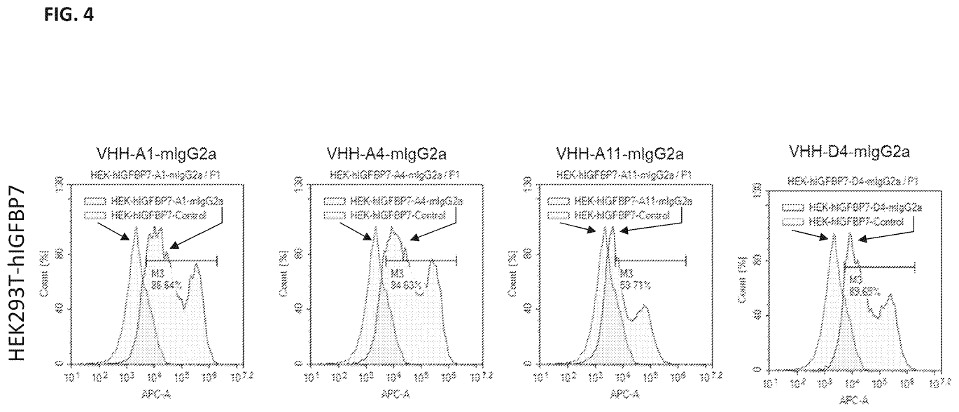

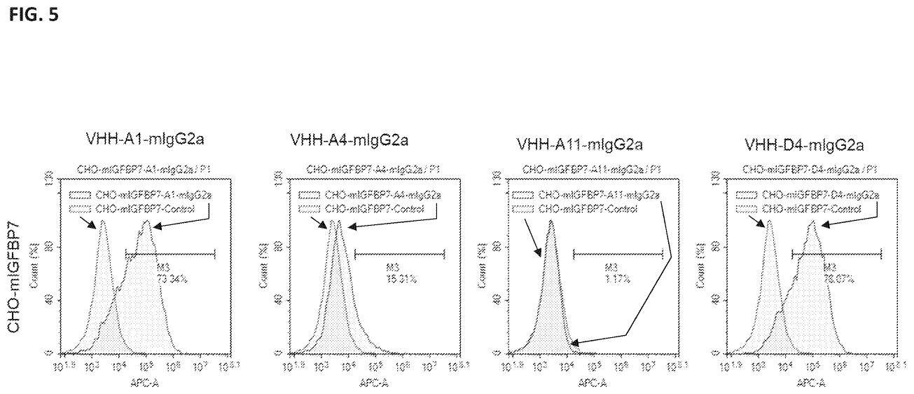

Initial screening results of the nanobody Fc-fusion constructs by fluorescence activated cell sorting (FACS) are shown in FIGS. 4 and 5. As shown in FIG. 4, the tested anti-IGFBP7 nanobody Fc-fusion constructs all bound to hIGFBP7-expressing HEK293T cells. FIG. 5 shows that tested anti-IGFBP7 nanobody Fc-fusion constructs derived from A1, A4 and D4 bound to mIGFBP7-expressing CHO-K1 cells.

### Example 9. Binding Affinity of Anti-IGFBP7-mIgG2a Fc to Human, Cynomolgus, and Mouse IGFBP7 Determined by a Bio-Layer Interferometry (BLI) Assay

The binding affinity of anti-IGFBP7 nanobody Fc-fusion constructs (e.g., VHH-Fc-fusion constructs) were determined with bio-layer interferometry (BLI) using Octet QKe (Fortebio). Human, mouse, and cynomolgus IGFBP7 were biotinylated using EZ-LINK NHS-PEG4 biotin (Thermo Fisher Scientific). Streptavidin biosensors (ForteBio) were used to load biotinylated IGFBP7 protein (300 seconds in 5 μg/mL). Baseline was stabilized for 60 seconds in 1× kinetics buffer (ForteBio) before anti-IGFBP7 Fc-fucion proteins (serial dilution) were allowed to associate for 300 seconds with captured hIGFBP7 or mIGFBP7. Then the sensors were dissociated in 1× kinetics buffer for 600 seconds. Data analysis was performed on ForteBio Data Analysis HT 11.1 software.

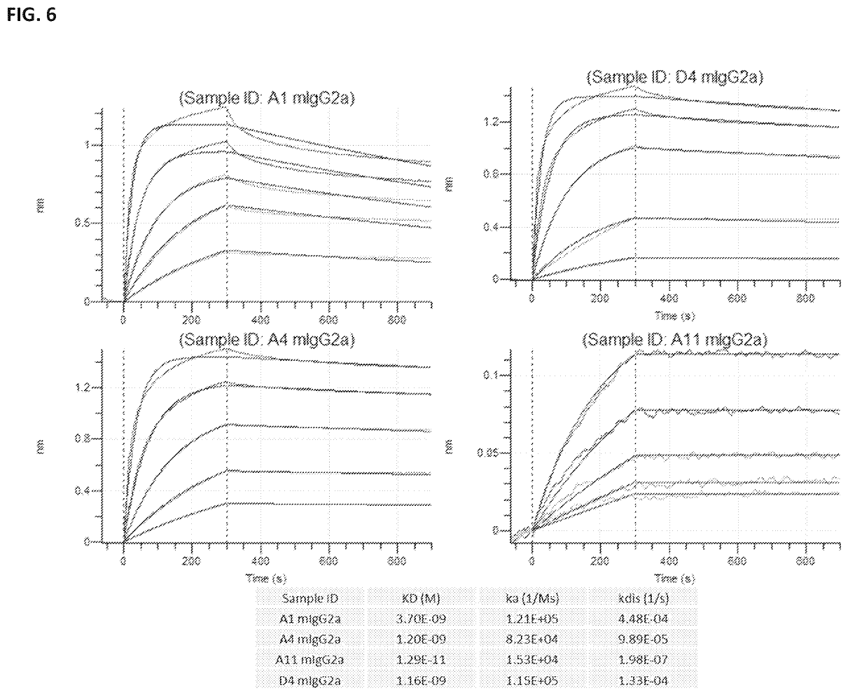

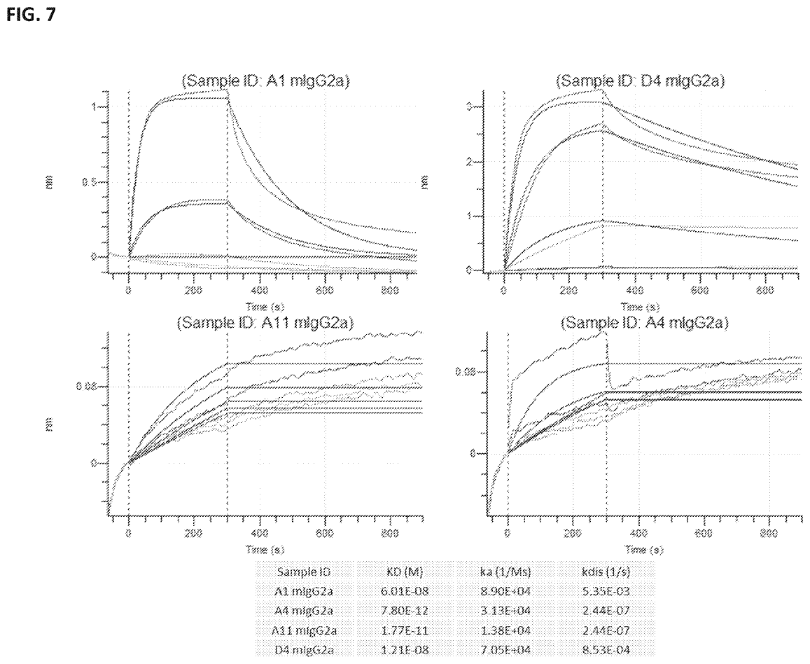

Results are shown in FIGS. 6 and 7. As shown, the tested nanobody Fc-fusion constructs bind to human and mouse IGFBP7.

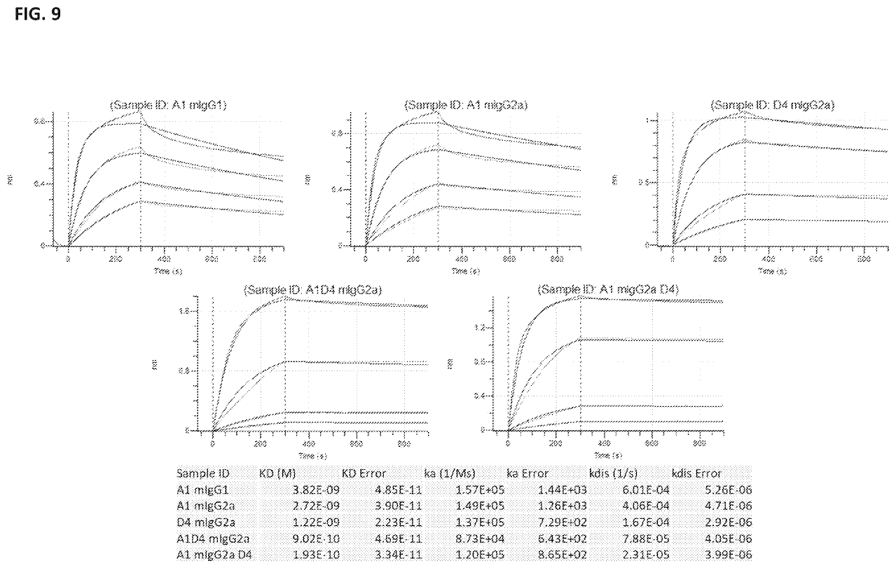

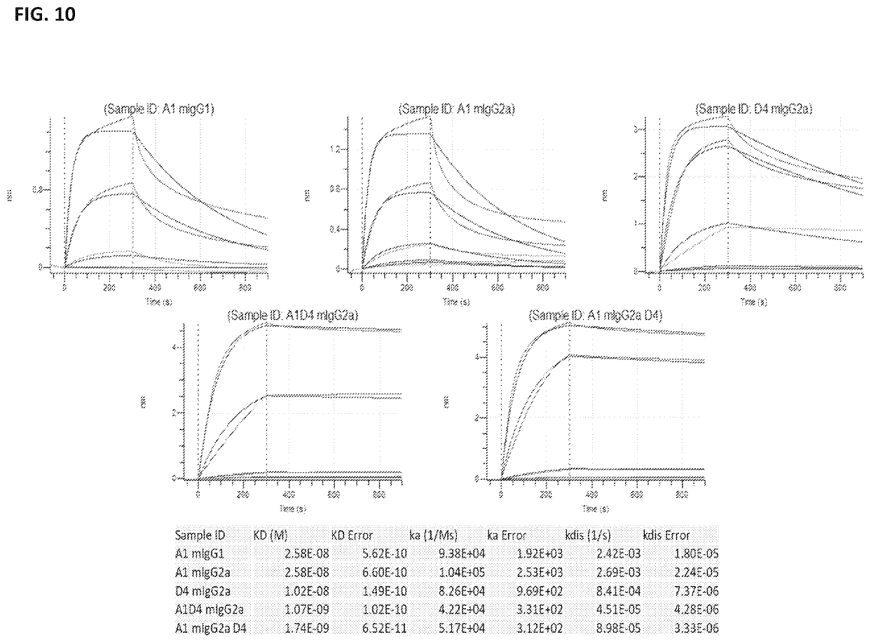

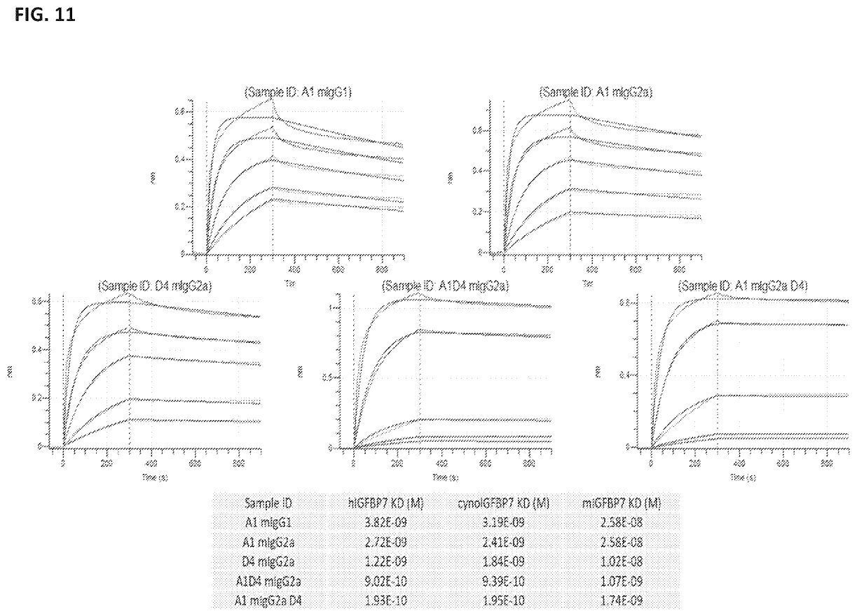

Mouse IgG bispecific anti-IGFBP7 nanobody Fc-fusion constructs such as exemplary bispecific A1-D4 mIgG2a and A1 mIgG2a-D4 as shown in FIG. 8 were also tested for their binding affinity to human, cynomolgus, and mouse IGFBP7 determined by the BLI assay. Results are shown in FIGS. 9-11. As shown, bispecific nanobody Fc-fusion constructs A1D4-mIgG2a and A1-mIgG2a-D4 have similar binding affinity for human, cynomolgus, and mouse IGFBP7 compared to monospecific A1-mIgG1, A1-mIgG2a, and D4-mIgG2a.

### Example 10. Binding of Anti-IGFBP7-mIgG2a Fc to Human and Mouse IGFBP7 Anchored to HEK293T Cells Determined by Fluorescence Activated Cell Sorting (FACS)

A stable pool of HEK 293T cells was made expressing human or mouse IGFBP7 with a linker and a CD80 transmembrane domain, to evaluate the anti-IGFBP7 nanobody Fc-fusion proteins binding to IGFBP7 in solution. hIGFBP7-HEK cells or mIGFBP7-HEK cells (1×10⁵ per well) were treated with anti-IGFBP7-mIgG2a Fc (75, 150, 300, and 600 nM) for 30 minutes at 4° C. After washing with FACS buffer, the cells were incubated with Alexa Fluor 488 conjugated anti-mouse IgG antibody (Jackson ImmunoResearch) for 30 minutes in 4° C. After washing with FACS buffer twice, the samples were acquired in NovoCyte Flow Cytometer and analyzed by NovoExpress software.

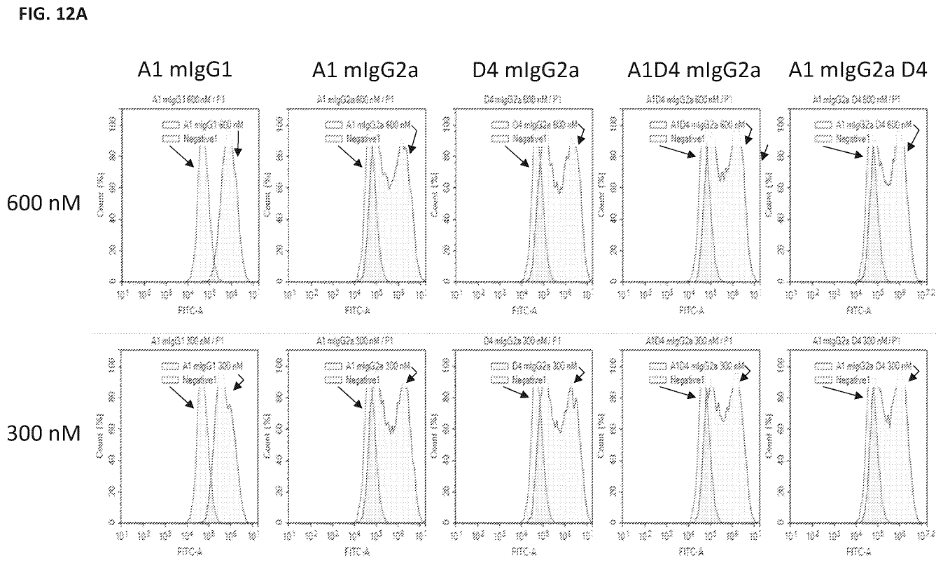

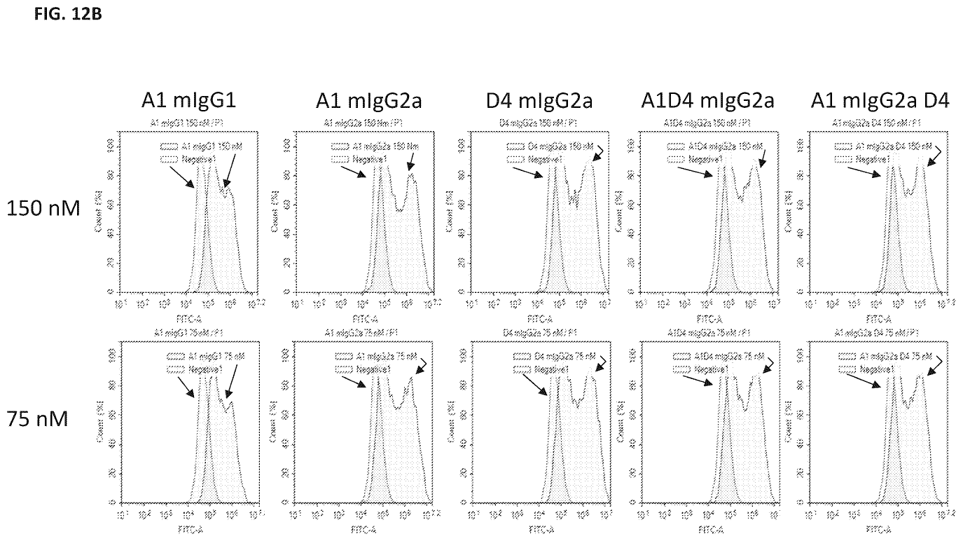

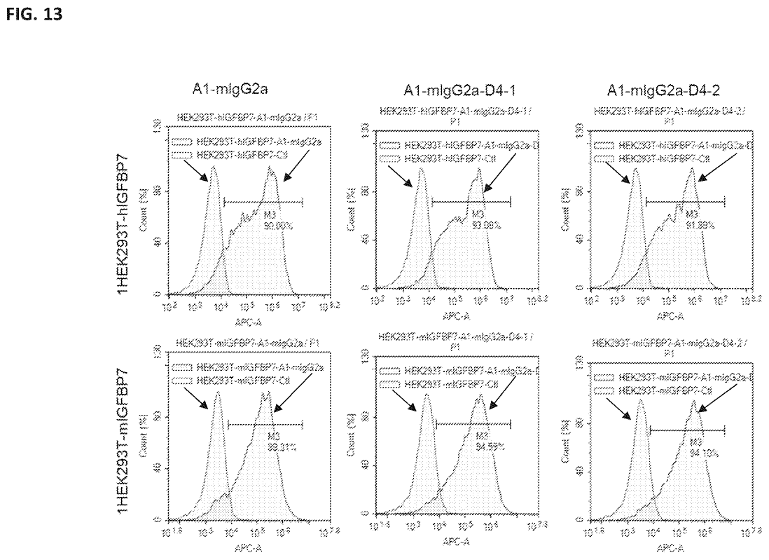

The results are shown in FIGS. 12A-12B and FIG. 13. As shown in FIGS. 12A-12B, the tested monospecific and bispecific anti-IGFBP7 nanobody Fc-fusion constructs bind to hIGFBP7 anchored to HEK293T cell surface at various concentrations. Further, FIG. 13 shows that bispecific A1-mIgG2a-D4-1 (i.e., A1D4-mIgG2a) and A1-mIgG2a-D4-2 (i.e., A1-mIgG2a-D4) bind to hIGFBP7-expressing or mIGFBP7-expressing HEK cells similarly as monoclonal A1-mIgG2a.

### Example 11. IGFBP7/CD93 Blockade Assay in Human CD93 Expressing CHO Cells by Anti-IGFBP7-mIgG2a Treatment

Anti-IGFBP7 nanobodies and nanobody-Fc fusions (50 μg/mL) generated in previous examples was incubated with His-tagged human IGFBP7 recombinant protein (1 μg/mL) for 30 minutes in 4° C. Human CD93 expressing CHO cells (1×10⁵ per well) were treated the mixture for 30 minutes at 4° C. Then the cells were washed with FACS buffer and incubated with anti-IGFBP7 monoclonal rabbit antibody (Sino Biological Inc, Catalog #13100-R003) at 1 μg/mL for 30 minutes in 4° C. After incubation, the cells were washed with FACS buffer and incubated with PE-conjugated anti-rabbit IgG antibody (Biole55gend) for 30 minutes in 4° C. After washing with FACS buffer twice, the samples were analyzed and data acquired in NovoCyte Flow.

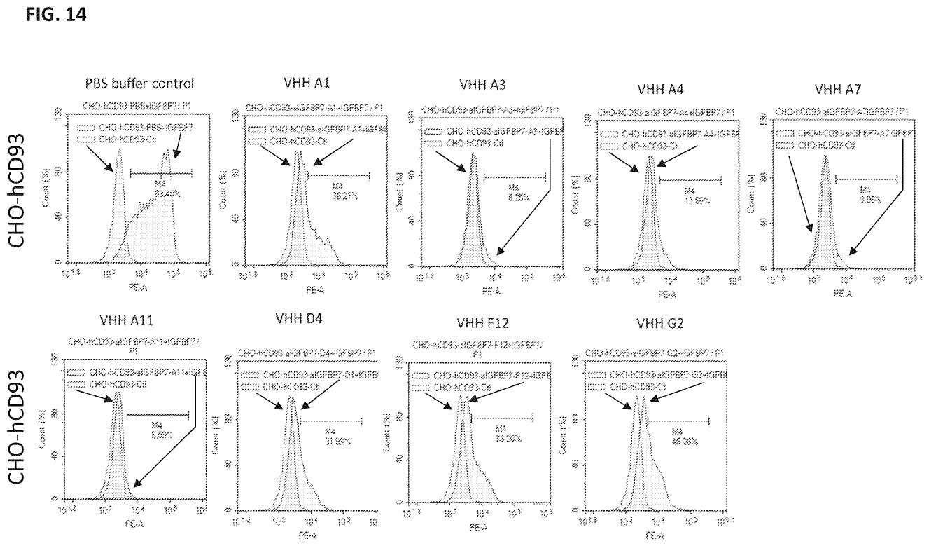

The results of exemplified anti-IGFBP7 nanobodies are shown in FIG. 14. As shown, all tested nanobodies effectively block the interaction between CD93 and IGFBP7 by at least 50%. Particularly, A3, A4, A7, and A11 almost completely blocked the interaction between CD93 and IGFBP7.

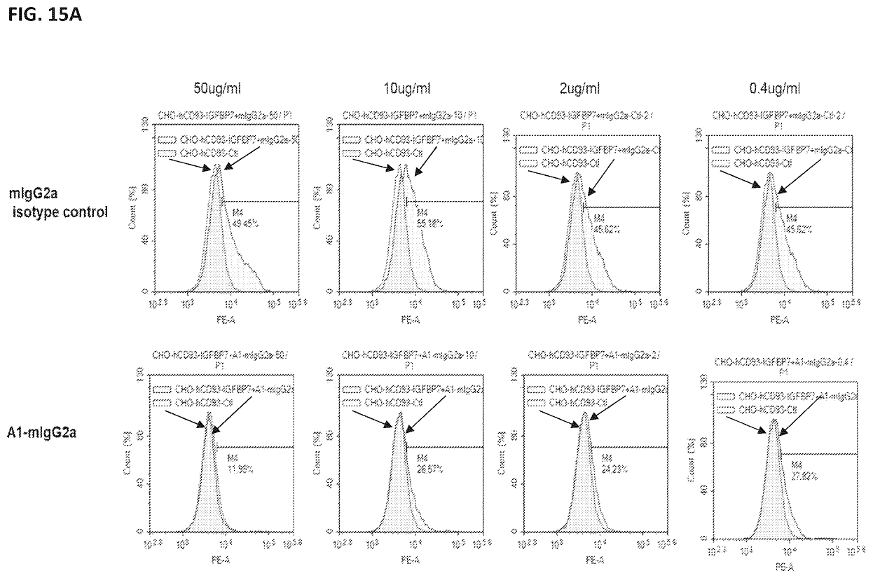

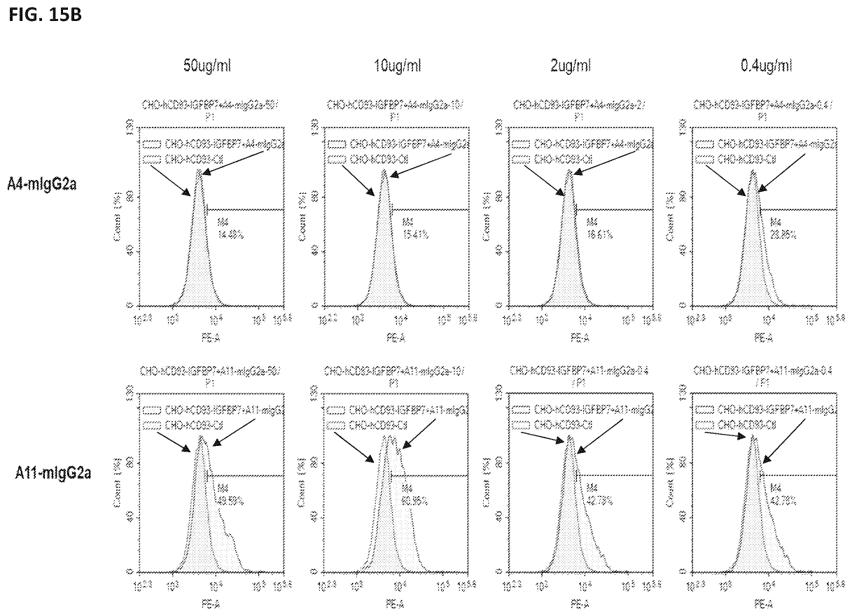

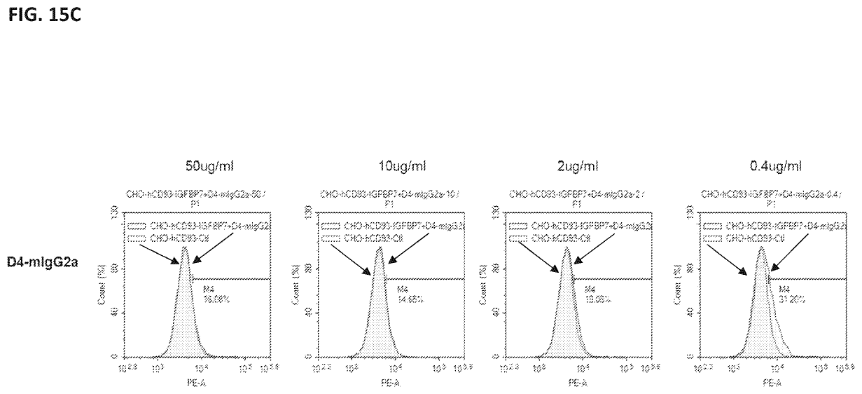

Mouse IgG anti-IGFBP7 nanobody Fc-fusion generated in Example 8 were also tested for their ability to block the interaction between IGFBP7 and CD93. Results are shown in FIGS. 15A-15C. As shown, A1-mIgG2a, A4-mIgG2a and D4-mIgG2a effectively block the interaction between IGFBP7 and CD93 at various concentrations.

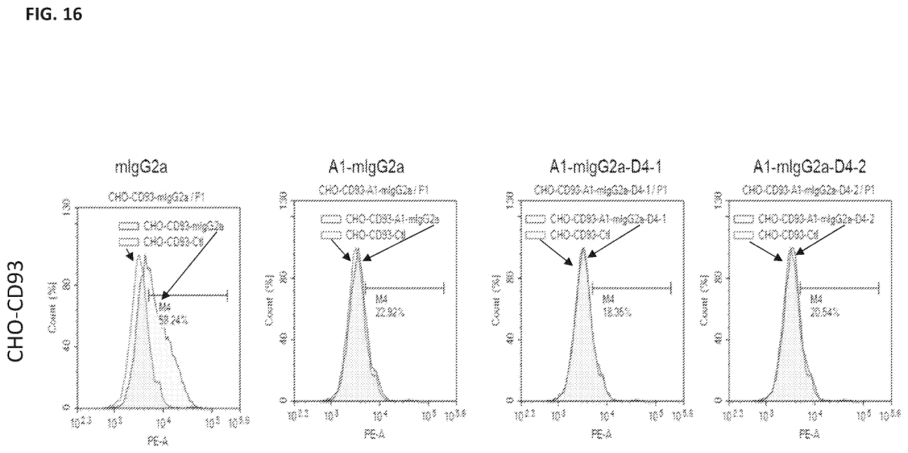

Exemplary bispecific anti-IGFBP7 nanobody Fc-fusion constructs (FIG. 8) were also tested for their ability to block the interaction between IGFBP7 and CD93. Results are shown in FIG. 16. As shown, bispecific antibodies A1-mIgG2a-D4-1 (i.e., A1D4-mIgG2a) and A1-mIgG2a-D4-2 (i.e., A1-mIgG2a-D4) have similar blocking activities.

### Example 12. HUVEC Tube Forming Inhibition Assay

Human umbilical vein endothelial cell (HUVECs, Thermo Fisher Scientific, Waltham, MA) were cultured in medium 200 supplemented with low serum growth supplement (LSGS, Thermo Fisher Scientific, Waltham, MA) at 37° C. with 5% CO₂. 96-well plates were coated with 50 μL of Geltrex reduced growth factor basement membrane matrix (Thermo Fisher Scientific) and incubated for 30 minutes at 37° C. To investigate the effects of anti-IGFBP7 mIgG2a fusion proteins on tube formation, 2×10⁴ HUVEC cells were seeded onto Matrix-coated plates and incubated in the presence or absence of purified fusion proteins for 18 hours at 37° C. with 5% CO₂. Images were obtained using a light microscope.

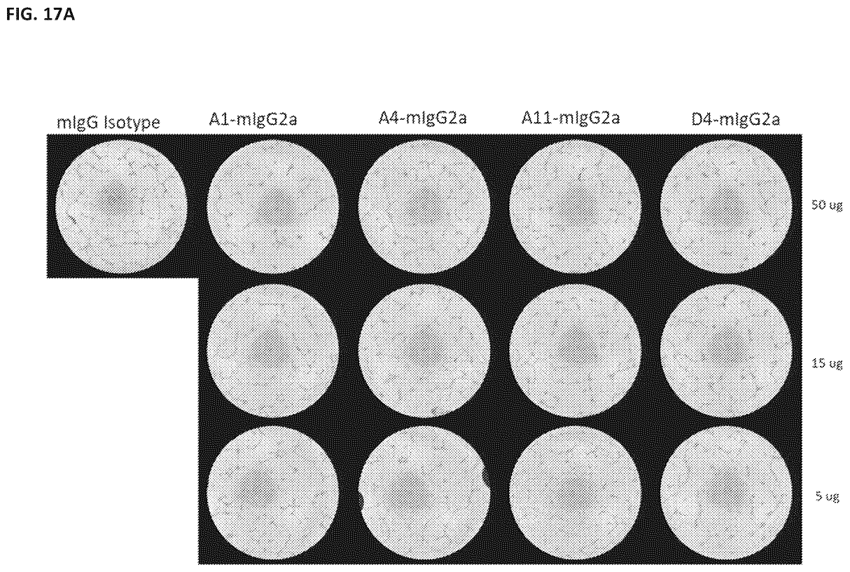

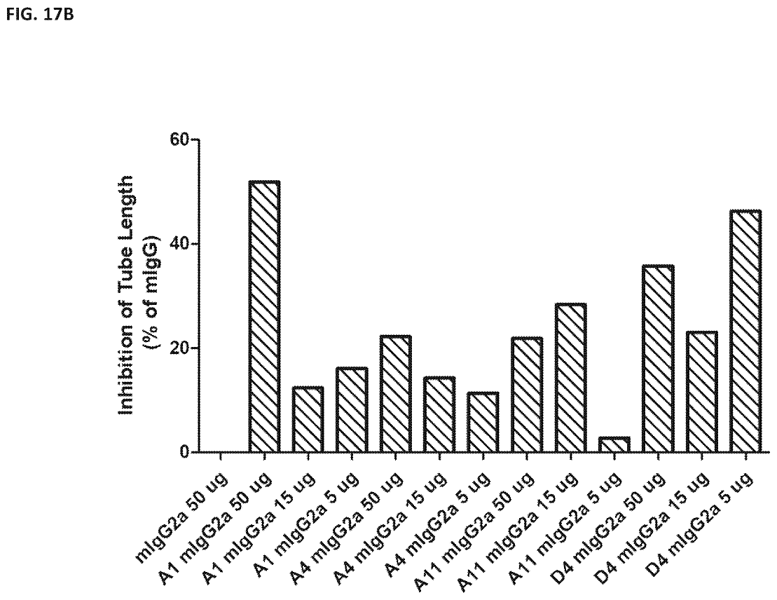

As shown in FIGS. 17A-17B, A1-mIgG2a, A4-mIgG2a, A11-mIgG2a, and D4-mIgG2a effectively inhibit tube formation of HUVEC cells at various concentrations, compared to a mIgG isotype control.

### Example 13. Epitope Binning Assay of Anti-IGFBP7 Antibodies by Octet Competition

Anti-IGFBP7 antibody epitope bins were determined using Octet QKe (ForteBio). Human IGFBP7 recombinant protein was biotinylated using EZ-LINK NHS-PEG4 biotin (Thermo Fisher Scientific). Streptavidin biosensors tips (ForteBio) were used to capture the biotinylated IGFBP7 protein (300 seconds at 5 μg/mL). A baseline measurement was stabilized for 60 seconds in 1× kinetics buffer (Fortebio) before primary anti-IGFBP7 antibodies (10 g/mL) were allowed to associate for 300 seconds with captured protein. A panel of secondary anti-IGFBP7 antibodies (10 μg/mL) were then allowed to associate with the antigen and primary antibody complex for an additional 300 seconds. Signals were recorded for each binding event and data analysis was performed on ForteBio Data Analysis HT 11.1 software.

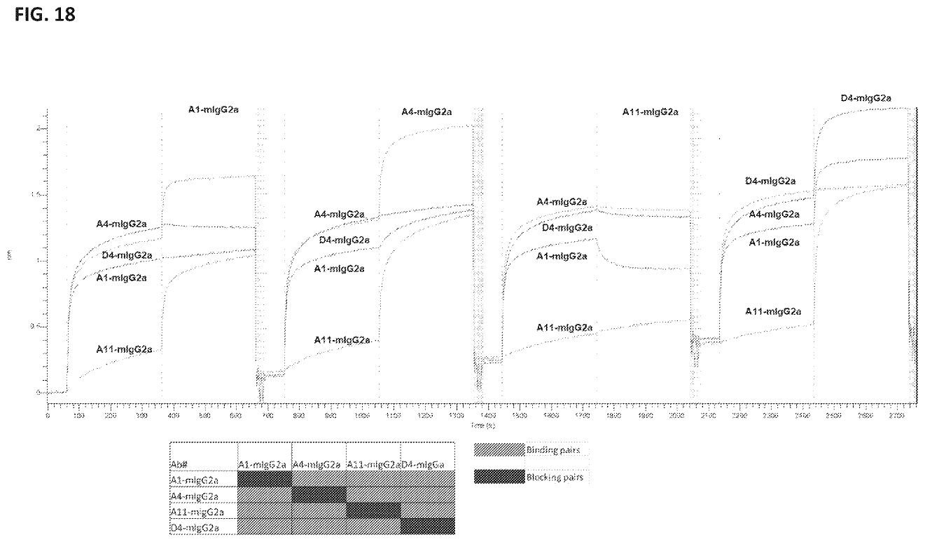

The epitope binning assay of anti-IGFBP7 antibodies via Octect analysis in FIG. 18 shows that A1, A4, A11, and D4 bind to different epitopes on IGFBP7.

### Example 14. In Vivo Therapeutic Efficacy of Anti-IGFBP7 Antibodies in a Tumor Model

A syngeneic KPC Pancreatic cancer model in female C57BL/6J mice was used to evaluate the in vivo therapeutic efficacy of anti-IGFBP7 antibody discussed above.

Seven-weeks old female C57BL/6J mice were subcutaneously injected with KPC tumor cells (2.0×10⁶) in 0.1 mL serum-free medium with 1:1 Matrigel in the right hind flank for tumor development. Tumor-bearing animals were randomized into 3 study groups of 6 or 7 mice each when the mean tumor size reached approximately 50 mm³. Included groups were mouse isotype control IgG2A, A1 mIgG2a-D4 (see Example 9 and FIG. 8). Each animal in all the groups received 0.3 mg/mouse of the respective articles by IP injection on days 0, 3, 7, 10. Tumor volume (TV) and body weight (BW) were measured and recorded twice a week until the study endpoint (Day 28) and individual animals were euthanized, tumors extracted, photographed and weighed (TW) for comparative analysis. Repeated Measure (RM) two-way ANOVA with Geisser-Greenhouse correction with Dunnett's multiple comparison test was performed to compare mean TVs to that of the isotype control at the endpoint. A Kruskal-Wallis test with Dunn's multiple comparison test was performed to compare mean TWs of treatment groups to that of the isotype control at the endpoint.

As shown in Table 5 below, the mice treated with A1 mIgG2a-D4 exhibited a consistent about 40% to 60% decrease in tumor volumes from Day 3 to Day 14 as compared to the mice treated with isotype control, suggesting the high effectiveness of anti-IGFBP7 antibodies in inhibiting tumor growth. After that, the tumor inhibition effects became less obvious, likely because A1 mIgG2a-D4 was cleared out from the circulation after their last administration at Day 10.

The overall body weight of the animals were stable in all groups throughout the study (data not shown), indicating that the treatments were well tolerated.

TABLE 5
Mean Tumor Volumes of two Groups
in Syngeneic KPC + C57BL/6J Model

| | Tumor Volumes (mm³) | | | | | |
|---|---|---|---|---|---|---|
| | Isotype control - Mouse | | | | | |
| | IgG2a | | | A1 mIgG2a-D4 | | |
| Days ¹ | Mean | SEM | N | Mean | SEM | N |
| 0 | 48.05 | 2.04 | 6 | 47.17 | 2.52 | 7 |
| 3 | 55.35 | 2.38 | 6 | 30.50 | 6.61 | 7 |
| 7 | 71.34 | 1.35 | 6 | 44.81 | 10.63 | 7 |
| 10 | 138.61 | 17.80 | 6 | 64.66 | 13.00 | 7 |
| 14 | 235.09 | 43.81 | 6 | 109.04 | 17.95 | 7 |
| 17 | 290.10 | 47.76 | 6 | 251.31 | 49.61 | 7 |
| 20 | 430.78 | 49.88 | 6 | 397.00 | 72.32 | 7 |
| 24 | 557.21 | 145.87 | 6 | 500.12 | 84.26 | 7 |
| 28 | 1295.09 | 271.48 | 6 | 1221.95 | 185.85 | 7 |

¹ Days following the start of treatment. Day 0 = day of treatment initiation

SEQUENCE TABLE

| SEQ ID | | |
|---|---|---|
| NO. | Description | Amino Acid Sequence |
| 1. | G02 CDR1 | NANKGAMT |
| 2. | A01 CDR1; | IDKWG |
| | hA1-1 CDR1; | |
| | hA1-2 CDR1; | |
| | hA1-3 CDR1 | |
| 3. | G02 CDR2; | WRYSPVSINYGDSVKG |
| | A01 CDR2; | |
| | hA1-1 CDR2; | |
| | hA1-2 CDR2; | |
| | hA1-3 CDR2 | |
| 4. | G02 CDR3; | VISGAWY |
| | A01 CDR3; | |
| | hA1-1 CDR3; | |
| | hA1-2 CDR3; | |
| | hA1-3 CDR3 | |
| 5. | D04 CDR1; | FSSYAMG |
| | hD4-1 CDR1; | |
| | hD4-2 CDR1; | |
| | hD4-3 CDR1 | |
| 6. | D04 CDR2; | AITWSGDYTYYTDSVKG |
| | hD4-1 CDR2; | |
| | hD4-2 CDR2; | |
| | hD4-3 CDR2 | |
| 7. | D04 CDR3; | KFSQVSAFFATVSEYEY |
| | hD4-1 CDR3; | |
| | hD4-2 CDR3; | |
| | hD4-3 CDR3 | |
| 8. | F12 CDR1 | INTYL |
| | A03 CDR1; | |
| | A04 CDR1 | |
| 9. | F12 CDR1 | INTYL |
| | A03 CDR1; | |
| | A04 CDR1 | |
| 10. | A07 CDR1 | AINGAM |
| 11. | A11 CDR1 | ANNGAM |
| 12. | F12 CDR2; | AITSGGSINYADSVKG |
| | A03 CDR2; | |
| | A04 CDR2; | |
| | A07 CDR2; | |
| | A11 CDR2 | |
| 13. | F12 CDR3; | KAHPNPWGFDNDY |
| | A03 CDR3; | |
| | A04 CDR3; | |
| | A07 CDR3; | |
| | A11 CDR3 | |
| 14. | B01 CDR1 | INAMA |
| 15. | B01 CDR2 | SLKYDGSTNYADFAKG |
| 16. | B01 CDR3 | DMTDWGLVAGQFDV |
| 17. | F09 CDR1 | INAMG |
| 18. | F09 CDR2 | ALTSGGSTMYGDSVKG |
| 19. | F09 CDR3 | RLIRTINGVDYDY |
| 20. | F02 CDR1 | LYAIA |
| 21. | F02 CDR2 | CISATHGEDIVAYRESVKG |
| 22. | F02 CDR3 | DGATCGDLYGRSWDGGPVNRPPQFGS |
| 23. | C03 CDR1 | AYAMG |
| 24. | C03 CDR2 | SISWTGSTSYYADSVKG |
| 25. | C03 CDR3 | TTYFRGTYNVESAFGS |
| 26. | A08 CDR1 | SYVMG |
| 27. | A08 CDR2 | AIPWDDDWTRYEDFVSG |
| 28. | A08 CDR3 | STRPGAPYVY |
| 29. | E12 CDR1 | TYYMT |
| 30. | E12 CDR2 | AINTGGGTTYYADSVKG |
| 31. | E12 CDR3 | GDYYESTYYPRY |
| 32. | A01 | VQLQESGGGLVQAGGSLRLSCAASGSLFAIDKWGWYR |
| | | QVAGNERELVAWRYSPVSINYGDSVKGRFTVSGDSSR |
| | | KRVYLQMNSLKPDDTAVYRCAAVISGAWYWGQGTQV |
| | | TVSS |
| 33. | G02 | VQLQESEGGWSGGGSASSDFDYDKMANANKGAMTGN |
| | | ADDAGNERELVAWRYSPVSINYGDSVKGRFTVSGDSS |
| | | RKRVYLQMNSLKPDDTAVYRCAAVISGAWYWGQGTQ |
| | | VTVSS |
| 34. | D04 | VQLQESGGGLVQTGGSLRLSCAASGRSFTFSSYAMGWF |
| | | RHAPGKEREFLTAITWSGDYTYYTDSVKGRSTISRDNA |
| | | KNTVYLQLNNLKPEDTAVYYCAAKFSQVSAFFATVSE |
| | | YEYWGQGTQVTVSS |
| 35. | F12 | VQLQESGGGLVQPGGSLRLSCAASGFTFSINTYLWYRQ |
| | | PPGKQREFVAAITSGGSINYADSVKGRFTISRDNAKKTV |
| | | YLQMNSLKPEDTAVYVCNIKAHPNPWGFDNDYWGQG |
| | | TQVTVSS |
| 36. | A03 | VQLQESGGGLVQAGGSLRLSCAASGTFSINTYLWYRQP |
| | | PGKQREFVAAITSGGSINYADSVKGRFTISRDNAKKTV |
| | | YLQMNSLKPEDTAVYVCNIKAHPNPWGFDNDYWGQG |
| | | TQVTVSS |
| 37. | A04 | VQLQESGGGLVQPGGSLRLSCAASGTFTINTYLWYRQP |
| | | PGKQREFVAAITRGGSINYADSVKGRFTISRDNAKKTV |
| | | YLQMNSLKPEDTAVYVCNIKAHPNPWGFDNDYWGQG |
| | | TQVTVSS |
| 38. | A07 | VQLQESEGGWSGGGSGSGDFGYDKMANAINGAMTDN |
| | | ADAGKQREFVAAITSGGSINYADSVKGRFTISTDNAKK |
| | | TVYLQMNSLKPEDTAVYVCNIKAHPNPWGFDNDYWG |
| | | QGTPATASS |
| 39. | A11 | VQLQESEGGWSGGGSGSGDFDYEKMANANNGAMTDN |
| | | ADAGKQREFVAAITSGGSINYADSVKGRFTISRDNTKK |
| | | TVYLQMNSLKPEDTAVYVCNIKAHPNPWGFDNDYWG |
| | | QGTQVTVSS |
| 40. | B01 | VQLQESGGGEVQPGGSLRLSCAASGHSFNINAMAWFR |
| | | QAPDKQRELVASLKYDGSTNYADFAKG |
| | | RFTISRGDAKDSIVLQLNSLKPEDTAVYRCAADMTDWG |
| | | LVAGQFDVWGRGTQVTVSS |
| 41. | F09 | VQLQESGGGLVQPGGSLRLSCSASGSIFSINAMGWYRQ |
| | | APGKQRELVAALTSGGSTMYGDSVKGRFTISRDGSKK |
| | | MVYLQMDSLKPEDTAVYYCASRLIRTINGVDYDYRGQ |
| | | GTQVTVSS |
| 42. | F02 | VQLQESGGGLVQPGGSLRLSCTAPGVTLDLYAIAWFRQ |
| | | APGKEREGVSCISATHGEDIVAYRESVKGRFTIFRDKTE |
| | | NAVYLQMNNLRPEDTAVYYCATDGATCGDLYGRSW |
| | | DGGPVNRPPQFGSWGQGTQVTVSS |
| 43. | C03 | VQLQESGGGSVQAGDSLTLSCSASGPAGSAYAMGWFR |
| | | QPPGKEREFVASISWTGSTSYYADSVKGRFSVSIVNAN |
| | | NTMLLEMKSLKPEDTAVYVCAATTYFRGTYNVESAFG |
| | | SWGQGKQVTVSS |
| 44. | A08 | VQLQESGGGLVQTGGSLRLSCAASGRTTSSYVMGWFR |
| | | QAPGKGRVFVAAIPWDDDWTRYEDFVSGRFTISIDNA |
| | | KNTVYLQMNSLKPDDTAVYYCAASTRPGAPYVYWGQ |
| | | GTQVTVSS |
| 45. | E12 | VQLQESGGGLVQPGGSLRLSCAASGFTFSTYYMTWVR |
| | | QAPGKGLEWISAINTGGGTTYYADSVKGRFTISRDNAK |
| | | NTLYLQMNSLKPEDTAVYYCTIGDYYESTYYPRYWGQ |
| | | GTQVTVSS |
| 46. | hA1-1 | QVQLVESGGGLVQPGGSLRLSCAASGSLFAIDKWGWF |
| | | RQAPGKEREIVSWRYSPVSINYGDSVKGRFTISRDNSK |
| | | NTLYLQMNSLRAEDTAVYYCAAVISGAWYWGQGTLV |
| | | TVSS |
| 47. | hA1-2 | EVQLLESGGGLVQPGGSLRLSCAASGSLFAIDKWGWV |
| | | RQAPGKGLEWVSWRYSPVSINYGDSVKGRFTISRDNSK |
| | | NTLYLQMNSLRAEDTAVYYCAAVISGAWYWGQGTLV |
| | | TVSS |
| 48. | hA1-3 | QVQLQESGGGLVQPGGSLRLSCAASGSLFAIDKWGWF |
| | | RQAPGKEREIVSWRYSPVSINYGDSVKGRFTISGDNSK |
| | | NTLYLQMNSLRAEDTAVYRCAAVISGAWYWGQGTQV |
| | | TVSS |
| 49. | hD4-1 | QVQLVESGGGLVQPGGSLRLSCAASGRSFTFSSYAMG |
| | | WFRQAPGKEREIVSAITWSGDYTYYTDSVKGRFTISRD |
| | | NSKNTLYLQMNSLRAEDTAVYYCARKFSQVSAFFATV |
| | | SEYEYWGQGTLVTVSS |
| 50. | hD4-2 | EVQLLESGGGLQQPGGSLRLSCAASGRSFTFSSYAMGW |
| | | VRQAPGKGLEWVSAITWSGDYTYYTDSVKGRFTISRD |
| | | NSKNTLYLQMNSLRAEDTAVYYCARKFSQVSAFFATV |
| | | SEYEYWGQGTMVTVSS |
| 51. | hD4-3 | EVQLQESGGGLQQPGGSLRLSCAASGRSFTFSSYAMGW |
| | | VRQAPGKGLEWVSAITWSGDYTYYTDSVKGRFTISRD |
| | | NSKNTLYLQMNSLRAEDTAVYYCAAKFSQVSAFFATV |
| | | SEYEYWGQGTQVTVSS |

Exemplary consensus sequences of anti-IGFBP7 antibody moieties

| | | |
|---|---|---|
| 52. | Group B1- | INTYL |
| | CDR1 | |
| | (F12/A03/ | |
| | A04) | |
| 53. | Group B2- | AX₁NGAM |
| | CDR1 | X₁ = N or I |
| | (A07/A11) | |

Exemplary linkers

| | | |
|---|---|---|
| 54. | Linker | (G)ₙ, n>=1 |
| 55. | Linker | (GS)ₙ, 8>=n>=1 |
| 56. | Linker | (GSGGS)ₙ, 8>=n>=1 |
| 57. | Linker | (GGGGS)ₙ, 8>=n>=1 |
| 58. | Linker | (GGGS)ₙ, 8>=n>=1 |
| 59. | Linker | (GGGGS)₃ |
| 60. | Linker | (GGGGS)₆ |
| 61. | Linker | (GSTSGSGKPGSGEGS)ₙ |
| | | 3>=n>=1 |

Exemplary anti-PD-L1 antibody moiety sequences

| | | |
|---|---|---|
| 62. | CDR-H1 | DTYMY |
| 63. | CDR-H2 | RIDPANDNTKYAQKFQG |
| 64. | CDR-H3 | AKNLLNYFDY |
| 65. | CDR-L1 | RASQEISGYLS |
| 66. | CDR-L2 | ATSTLQS |
| 67. | CDR-L3 | LQYAIYPLT |
| 68. | Humanized | QVQLVQSGAEVKKPGASVKVSCKASGFNIKDTYMYWV |
| | VH 1 | RQAPGQGLEWMGRIDPANDNTKYAQKFQGRVTITADT |
| | | STSTAYMELSSLRSEDTAVYYCARAKNLLNYFDYWGQ |
| | | GTLVTVSS |
| 69. | Humanized | QVQLVQSGAEVKKPGASVKVSCKASGFNIKDTYMYWV |
| | VH 2 | RQAPGQGLEWIGRIDPANDNTKYAPKFQGRVTITADTST |
| | | NTAYMELSSLRSEDTAVYYCARAKNLLNYFDYWGQGT |
| | | LVTVSS |
| 70. | Humanized | EVQLVQSGAEVKKPGASVKVSCKASGFNIKDTYMYWV |
| | VH3 | RQAPGQGLEWMGRIDPANDNTKYAQKFQGRVTITADT |
| | | STNTAYMELSSLRSEDTAVYYCARAKNLLNYFDYWGQ |
| | | GTLVTVSS |
| 71. | Humanized | DIQMTQSPSSLSASVGDRVTITCRASQEISGYLSWYQQK |
| | VL 1 | PGKAPKRLIYATSTLDSGVPSRFSGSGSGTDFTLTISSLQP |
| | | EDFATYYCLQYAIYPLTFGQGTKLEIKR |
| 72. | Humanized | DIQMTQSPSSLSASVGDRVTITCRASQEISGYLSWLQQK |
| | VL 2 | PGKAPKRLIYATSTLQSGVPSRFSGSRSGTDYTLTISSLQ |
| | | PEDFATYYCLQYAIYPLTFGQGTKLEIKR |
| 73. | Humanized | DIQMTQSPSSLSASVGDRVTITCRASQEISGYLSWYQQK |
| | VL 3 | PGKAPKRLIYATSTLDSGVPSRFSGSRSGSDYTLTISSLQP |
| | | EDFATYYCLQYAIYPLTFGQGTKLEIKR |

Exemplary anti-PD-1 antibody moiety sequences

| | | |
|---|---|---|
| 74 | Ab1 CDR-H1 | GFTFSSYT |
| | (Vbase2) | |
| 75. | Ab1 CDR-H2 | ISHGGGDT |
| | (Vbase2) | |
| 76. | Ab1 CDR-H3 | ARHSGYERGYYYVMDY |
| | (Vbase2) | |
| 77. | Ab1 CDR-L1 | ESVDYYGFSF |
| | (Vbase2) | |
| 78. | Ab1 CDR-L2 | AAS |
| | (Vbase2) | |
| 79. | Ab1 CDR-L3 | QQSKEVPW |
| | (Vbase2) | |
| 80. | Ab2 CDR-H1 | GYTFTSYT |
| | (Vbase2) | |
| 81. | Ab2 CDR-H2 | INPTTGYT |
| | (Vbase2) | |
| 82. | Ab2 CDR-H3 | ARDDAYYSGY |
| | (Vbase2) | |
| 83. | Ab2 CDR-L1 | ENIYSNL |
| | (Vbase2) | |
| 84. | Ab2 CDR-L2 | AAK |
| | (Vbase2) | |
| 85. | Ab2 CDR-L3 | QHFWGTPWT |
| | (Vbase2) | |
| 86. | Ab3 CDR-H1 | GFAFSSYD |
| | (Vbase2) | |
| 87. | Ab3 CDR-H2 | ITIGGGTT |
| | (Vbase2) | |
| 88. | Ab3 CDR-H3 | ARHRYDYFAMDN |
| | (Vbase2) | |
| 89. | Ab3 CDR-L1 | ENVDNYGINF |
| | (Vbase2) | |
| 90. | Ab3 CDR-L2 | VSS |
| | (Vbase2) | |
| 91. | Ab3 CDR-L3 | QQSKDVPW |
| | (Vbase2) | |
| 92. | Murine Ab1 | SQVQLQQSGAELARPGASVKMSCKASGYTFTSYTMHW |
| | VH | VKQRPGQGLEWIGYINPTTGYTNYNQKFKDKANPTTGY |
| | | TNYNQKFKDKATLTADKSSSTAYMQLSSLTSEDSAVYY |
| | | CARDDAYYSGYWGQGTTLTVSS |
| 93. | Murine Ab1 | DIQMTQSPASLSVSVGETVTITCRASENIYSNLAWYROK |
| | VL | QGKSPQLLVYAAKNLADGVPSRFSGSGSGTQYSLKINSL |
| | | QSEDFGSYYCQHFWGTPWTFGGGTKLEIKR |
| 94 | Murine Ab2 | VQLVESGGGLVKPGGSLKLSCAASGFAFSSYDMSWVR |
| | VH | QTPEKRLVWVAYITIGGGTTYYSDTVKRLVWVAYITIG |
| | | GGTTYYSDTVKGRFTISRDNAKNTLYLQMSSLKSEDTA |
| | | MYYCARHRYDYFAMDNWGHGTSVTVSS |
| 95. | Murine Ab2 | DIVLTQSPASLAVSLEHRATISCQASENVDNYGINFMNW |
| | VL | FQHKPAQPPQLLIYVSSNLGSGVPAKFSGSGSGTDFSLNI |
| | | HPMEEDDTAMYFCQQSKDVPWTFSGGTKLEIKR |
| 96. | Murine Ab3 | EVKLVESGGGLVQPGGSLKLSCAASGFTFSSYTMSWIR |
| | VH | QTPEKRLEWVAYISHGGGDTYYPDTVKGRFTISRDNAK |
| | | NTLYLQMSSLKSEDTAMYYCARHSGYERGYYYVMDY |
| | | WGQGTSVTVSS |
| 97. | Murine Ab3 | DIVLTQFPTSLAVSLGQRATISCRASESVDYYGFSFINWF |
| | VL | QQKPGQPPKLLIYAASNQGSGVPARFGGSGSGTDFSLNI |
| | | HPMEEDDTAMYFCQQSKEVPWTFGGGTKLEIK |

Exemplary anti-VEGF sequences

| | | |
|---|---|---|
| 98. | Afibercept | SDTGRPFVEMYSEIPEIIHMTEGRELVIPCRVTSPNITVTL |
| | | KKFPLDTLIPDGKRIIWDSRKGFIISNATYKEIGLLTCEAT |
| | | VNGHLYKTNYLTHRQTNTIIDVVLSPSHGIELSVGEKLV |
| | | LNCTARTELNVGIDENWEYPSSKHQHKKLVNRDLKTQS |
| | | GSEMKKFLSTLTIDGVTRSDQGLYTCAASSGLMTKKNS |
| | | TFVRVH |
| 99. | Avastin HC- | GYTFTNYGMN |
| | CDR1 (Kabat) | |
| 100. | Avastin HC- | WINTYTGEPTYAADFKR |
| | CDR2 (Kabat) | |
| 101. | Avastin HC- | YPHYYGSSHWYFDV |
| | CDR3 (Kabat) | |
| 102. | Avastin LC- | SASQDISNYLN |
| | CDR1 (Kabat) | |
| 103. | Avastin LC- | FTSSLHS |
| | CDR2 (Kabat) | |
| 104. | Avastin LC- | QQYSTVPWT |
| | CDR3 (Kabat) | |
| 105. | Ramucirumab | SYSMN |
| | HC-CDR1 | |
| | (Kabat) | |
| 106. | Ramucirumab | SISSSSSYIYYADSVKG |
| | HC-CDR2 | |
| | (Kabat) | |
| 107. | Ramucirumab | VTDAFDI |
| | HC-CDR3 | |
| | (Kabat) | |
| 108. | Ramucirumab | RASQGIDNWLG |
| | LC-CDR1 | |
| | (Kabat) | |
| 109. | Ramucirumab | DASNLDT |
| | LC-CDR2 | |
| | (Kabat) | |
| 110. | Ramucirumab | QQAKAFPPT |
| | LC-CDR3 | |
| | (Kabat) | |

Exemplary IGFBP7 sequence

| | | |
|---|---|---|
| 111. | Human | MERPSLRALLLGAAGLLLLLLPLSSSSSSDTCGPCEPASC |
| | IGFBP7 | PPLPPLGCLLGETRDACGCCPMCARGEGEPCGGGGAGR |
| | | GYCAPGMECVKSRKRRKGKAGAAAGGPGVSGVCVCK |
| | | SRYPVCGSDGTTYPSGCQLRAASQRAESRGEKAITQVSK |
| | | GTCEQGPSIVTPPKDIWNVTGAQVYLSCEVIGIPTPVLIW |
| | | NKVKRGHYGVQRTELLPGDRDNLAIQTRGGPEKHEVT |
| | | GWVLVSPLSKEDAGEYECHASNSQGQASASAKITVVDA |
| | | LHEIPVKKGEGAEL |

Alternative CDR definitions with more AA covered CDRs sequences

| | | |
|---|---|---|
| 112. | A01 CDR1; | GSLFAIDKWG |
| | hA1-1 CDR1; | |
| | hA1-2 CDR1; | |
| | hA1-3 CDR1 | |
| 113. | G02 CDR3; | AAVISGAWY |
| | A01 CDR3; | |
| | hA1-1 CDR3; | |
| | hA1-2 CDR3; | |
| | hA1-3 CDR3 | |
| 114. | D04 CDR1; | GRSFTFSSYAMG |
| | hD4-1 CDR1; | |
| | hD4-2 CDR1; | |
| | hD4-3 CDR1 | |
| 115. | D04 CDR3; | AKFSQVSAFFATVSEYEY |
| | hD4-3 CDR3 | |
| 116. | hD4-1 CDR3; | ARKFSQVSAFFATVSEYEY |
| | hD4-2 CDR3 | |

1. An anti-IGFBP7 construct comprising a polypeptide comprising a single domain antibody (sdAb) moiety specifically recognizing IGFBP7, wherein the sdAb moiety comprises:
1) a CDR1 comprising the amino acid sequence of SEQ ID NO: 1, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
2) a CDR1 comprising the amino acid sequence of SEQ ID NO: 2 or 112, a CDR2 comprising the amino acid sequence of SEQ ID NO: 3, and a CDR3 comprising the amino acid sequence of SEQ ID NO: 4 or 113, or a variant thereof comprising up to 5, 4, 3, 2, or 1 amino acid substitutions in the CDRs;
3) a CDR1 comprising the amino acid sequence of SEQ ID NO: 5 or 114, a CDR2 comprising the amino acid sequence of SEQ ID NO: 6, and a CDR3 comprising the amino acid sequence of SEQ ID NO: 7, 115 or 116, or a variant thereof comprising up to 5, 4, 3, 2, or 1 amino acid substitutions in the CDRs;
4) a CDR1 comprising the amino acid sequence of SEQ ID NO: 8, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
5) a CDR1 comprising the amino acid sequence of SEQ ID NO: 9, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
6) a CDR1 comprising the amino acid sequence of SEQ ID NO: 10, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
7) a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, or a variant thereof comprising up to 5, 4, 3, 2, or 1 amino acid substitutions in the CDRs;
8) a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, or a variant thereof comprising up to 5, 4, 3, 2, or 1 amino acid substitutions in the CDRs;
9) a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, or a variant thereof comprising up to 5, 4, 3, 2, or 1 amino acid substitutions in the CDRs;
10) a CDR1 comprising the amino acid sequence of SEQ ID NO: 20, a CDR2 comprising the amino acid sequence of SEQ ID NO: 21, and a CDR3 comprising the amino acid sequence of SEQ ID NO: 22, or a variant thereof comprising up to 5, 4, 3, 2, or 1 amino acid substitutions in the CDRs;
11) a CDR1 comprising the amino acid sequence of SEQ ID NO: 23, a CDR2 comprising the amino acid sequence of SEQ ID NO: 24, and a CDR3 comprising the amino acid sequence of SEQ ID NO: 25, or a variant thereof comprising up to 5, 4, 3, 2, or 1 amino acid substitutions in the CDRs;
12) a CDR1 comprising the amino acid sequence of SEQ ID NO: 26, a CDR2 comprising the amino acid sequence of SEQ ID NO: 27, and a CDR3 comprising the amino acid sequence of SEQ ID NO: 28, or a variant thereof comprising up to 5, 4, 3, 2, or 1 amino acid substitutions in the CDRs; or
13) a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, or a variant thereof comprising up to 5, 4, 3, 2, or 1 amino acid substitutions in the CDRs.

2. An anti-IGFBP7 construct comprising a polypeptide comprising a single domain antibody (sdAb) moiety specifically recognizing IGFBP7, wherein the sdAb moiety comprises the amino acid sequences of a CDR1, a CDR2, and a CDR3 within the amino acid sequence set forth in any of SEQ ID NOs: 32-51.

3. The anti-IGFBP7 construct of claim 1 or 2, wherein the sdAb moiety comprises the amino acid sequence of any one of SEQ ID NOs: 32-51, or a variant thereof having at least about 80% sequence identify to any one of SEQ ID NOs: 32-51.

4. The anti-IGFBP7 construct of any one of claims 1-3, wherein the sdAb moiety is camelid, chimeric, human, partially humanized, or fully humanized.

5. The anti-IGFBP7 construct of any one of claims 1-4, wherein the sdAb moiety is a VHH antibody.

6. The anti-IGFBP7 construct of any one of claims 1-5, wherein anti-IGFBP7 construct blocks the binding of CD93 to IGFBP7.

7. The anti-IGFBP7 construct of any one of claims 1-6, wherein the IGFBP7 is human IGFBP7.

8. The anti-IGFBP7 construct of claim 6 or claim 7, wherein the CD93 is human CD93.

9. The anti-IGFBP7 construct of any one of claims 1-8, wherein the anti-IGFBP7 construct further comprises a second moiety.

10. The anti-IGFBP7 construct of claim 9, wherein the second moiety comprises an antibody moiety that specifically recognizes an antigen.

11. The anti-IGFBP7 construct of claim 10, wherein the antigen is PD-L1 or PD-1.

12. The anti-IGFBP7 construct of claim 10 or 11, wherein the second antibody moiety is a full-length antibody, a Fab, a Fab′, a (Fab′)₂, an Fv, a single chain Fv (scFv), an scFv-scFv, a minibody, a diabody, or an sdAb.

13. The anti-IGFBP7 construct of claim 9, where the second moiety comprises a half-life extending moiety.

14. The anti-IGFBP7 construct of claim 9, wherein the construct is an antibody-drug conjugate.

15. An anti-IGFBP7 construct that specifically binds to IGFBP7 competitively with the anti-IGFBP7 construct of any one of claims 1-14.

16. A pharmaceutical composition comprising the anti-IGFBP7 construct of any one of claims 1-15 and a pharmaceutical acceptable carrier.

17. A polynucleotide encoding the polypeptide of the anti-IGFBP7 construct of any one of claims 1-15 or a portion thereof.

18. A nucleic acid construct, comprising the polynucleotide of claim 17, optionally further comprising a promoter in operative connection with the polynucleotide.

19. A vector comprising the nucleic acid construct of claim 18.

20. An isolated host cell comprising the polynucleotide according to claim 18, the nucleic acid construct according to claim 18, or the vector according to claim 19.

21. A culture medium comprising the polypeptide of anti-IGFBP7 construct of claim any one of claims 1-15, the polynucleotide according to claim 17, the nucleic acid construct according to claim 18, the vector according to claim 19, or the host cell according to claim 20.

22. A method of producing an anti-IGFBP7 construct, comprising:
a) culturing the isolated host cell of claim 20 under conditions effective to express the polypeptide; and
b) obtaining the polypeptide from the host cell.

23. A method of treating a disease or condition or inhibiting abnormal vessel growth in a tissue in an individual, comprising administering to the individual an effective mount of the anti-IGFBP7 construct of any one of claims 1-15, or the pharmaceutical composition of claim 16.

24. The method of claim 23, wherein the disease of condition is associated with an abnormal vascular structure.

25. The method of claim 23 or claim 24, wherein the disease or condition is a cancer.

26. The method of claim 25, wherein the cancer is a solid tumor.

27. The method of claim 25 or claim 26, wherein the cancer comprises CD93+ endothelial cells.

28. The method of any one of claims 25-27, wherein the cancer comprises IGFBP7+ blood vessels.

29. The method of any one of claims 25-28, wherein the cancer is characterized by tumor hypoxia.

30. The method of any one of claims 25-29, wherein the cancer is a locally advanced or metastatic cancer.

31. The method of any one of claims 25-30, wherein the cancer is selected from the group consisting of a lymphoma, colon cancer, breast cancer, ovarian cancer, endometrial cancer, esophageal cancer, prostate cancer, cervical cancer, renal cancer, bladder cancer, gastric cancer, non-small cell lung cancer, melanoma, and pancreatic cancer.

32. The method of any one of claims 23-31, wherein the anti-IGFBP7 construct is administered parenterally into the individual.

33. The method of any one of claims 23-32, wherein the method further comprises administering a second therapy.

34. The method of claim 33, wherein the second therapy is selected from the group consisting of surgery, radiation, gene therapy, immunotherapy, bone marrow transplantation, stem cell transplantation, hormone therapy, targeted therapy, cryotherapy, ultrasound therapy, photodynamic therapy, and chemotherapy.

35. The method of claim 34, wherein the second therapy is an immunotherapy.

36. The method of claim 35, wherein the immunotherapy comprises administering an immunomodulatory agent.

37. The method of claim 36, wherein the immunomodulatory agent is an immune checkpoint inhibitor.

38. The method of claim 37, wherein the immune checkpoint inhibitor comprises an anti-PD-L1 antibody or an anti-PD-1 antibody.

39. The method of any one of claims 23-38, wherein the individual is a human.
